(19) United States
(12) Patent Application Publication
(10) Pub. No.: US 2014/0212382 A1
(43) Pub. Date: Jul. 31, 2014

(54) PURINE MONOPHOSPHATE PRODRUGS FOR TREATMENT OF VIRAL INFECTIONS

(71) Applicants: Raymond F. Schinazi, Miami, FL (US); Jong Hyun Cho, Snellville, GA (US); Longhu Zhou, Atlanta, GA (US); Hongwang Zhang, Tucker, GA (US); Ugo Pradere, Zurich (CH); Steven J. Coats, McDonough, GA (US)

(72) Inventors: Raymond F. Schinazi, Miami, FL (US); Jong Hyun Cho, Snellville, GA (US); Longhu Zhou, Atlanta, GA (US); Hongwang Zhang, Tucker, GA (US); Ugo Pradere, Zurich (CH); Steven J. Coats, McDonough, GA (US)

(73) Assignees: EMORY UNIVERSITY, Atlanta, GA (US); RFS PHARMA, LLC, Tucker, GA (US)

(21) Appl. No.: 14/117,815

(22) PCT Filed: May 16, 2012
(86) PCT No.: PCT/US2012/038165

§ 371 Date: Apr. 8, 2014

Related U.S. Application Data
(60) Provisional application No. 61/488,140, filed on May 19, 2011.

(51) Int. Cl. C07F 9/6558; A61K 31/7076; A61K 45/06; C07F 9/6574

(52) U.S. Cl. CPC C07F 9/65586; C07F 9/65742; A61K 31/7076; A61K 45/06
USPC 424/85.4; 536/26.7; 536/26.13; 514/47

(57) ABSTRACT

The present invention is directed to compounds, compositions and methods for treating or preventing viral infections using nucleoside analog monophosphate prodrugs. More specifically, HCV, Norovirus, Saporovirus, Dengue virus, Chikungunya virus and Yellow fever in human patients or other animal hosts. The compounds are certain 2,6-diamino 2-C-methyl purine nucleoside monophosphate prodrugs and modified prodrug analogs, and pharmaceutically acceptable, salts, prodrugs, and other derivatives thereof. In particular, the compounds show potent antiviral activity against HCV, Norovirus, Saporovirus, Dengue virus, Chikungunya virus and Yellow fever. This invention teaches how to modify the metabolic pathway of 2,6-diamino 2′-C-methyl purine and deliver nucleotide triphosphate(s) to polymerases at heretofore unobtainable therapeutically-relevant concentrations.

### FIELD OF THE INVENTION

The present invention is directed to compounds, methods and compositions for treating or preventing viral infections using nucleotide analogs. More specifically, the invention describes 2,6-diamino 2′-C-Me purine nucleoside monophosphate prodrugs and modified prodrug analogs, pharmaceutically acceptable salts, or other derivatives thereof, and the use thereof in the treatment of viral infection(s), and in particular 1) Flaviviridae family of viruses including hepatitis C (HCV), West Nile virus, Dengue virus, Chikungunya virus and Yellow fever; and 2) Caliciviridae infection including Norovirus and Sapovirus. This invention teaches how to modify the metabolic pathway of 2,6-diamino 2′-C-methyl purines and deliver nucleotide triphosphates to polymerases at heretofore unobtainable therapeutically-relevant concentrations.

### BACKGROUND OF THE INVENTION

Nucleoside analogs as a class have a well-established regulatory history, with more than 10 currently approved by the US Food and Drug Administration (US FDA) for treating human immunodeficiency virus (HIV), hepatitis B virus (HBV), or hepatitis C virus (HCV). The challenge in developing antiviral therapies is to inhibit viral replication without injuring the host cell. In general, to exhibit antiviral activity, nucleoside analogs must be metabolically converted by host-cell kinases to their corresponding triphosphate forms (NTP). In the triphosphate form, nucleoside polyermase inhibitors mimic natural nucleotides as they compete with one of the five naturally occurring nucleoside 5′-triphosphates (NTP), namely, CTP, UTP, TTP, ATP, or GTP for RNA or DNA elongation. Thus nucleoside analogs inhibit viral replication by acting as chain terminators or delayed chain terminators.

Hepatitis C virus (HCV) has infected more than 180 million people worldwide. It is estimated that three to four million persons are newly infected each year, 70% of whom will develop chronic hepatitis. HCV is responsible for 50-76% of all liver cancer cases, and two thirds of all liver transplants in the developed world. Standard therapy [pegylated interferon alfa plus ribavirin (a nucleoside analog)] is only effective in 50-60% of patients and is associated with significant side-effects. Therefore, there is an urgent need for new HCV drugs.

Hepatitis C virus genome comprises a positive-strand RNA enclosed in a nucleocapsid and lipid envelope and consists of 9.6 kb ribonucleotides, which encodes a large polypeptide of about 3000 amino acids (Dymock et al. Antiviral Chemistry & Chemotherapy 2000, 11, 79). Following maturation, this polypeptide is cut into at least 10 proteins. One of these proteins, NS5B, possesses polymerase activity and is involved in the synthesis of double-stranded RNA from the single-stranded viral RNA genome that serves as the template. The discovery of novel antiviral strategies to selectively inhibit HCV replication has long been hindered by the lack of convenient cell culture models for the propagation of HCV. This hurdle has been overcome first with the establishment of the HCV replicon system in 1999 (Bartenschlager, R., Nat. Rev. Drug Discov. 2002, 1, 911-916 and Bartenschlager, R., J. Hepatol. 2005, 43, 210-216) and, in 2005, with the development of robust HCV cell culture models (Wakita, T., et al., Nat. Med. 2005, 11, 791-6; Zhong, J., et al., Proc. Natl. Acad. Sci. U.S.A. 2005, 102, 9294-9; Lindenbach, B. D., et al., Science 2005, 309, 623-6).

HCV replication may be prevented through the manipulation of NS5B's polymerase activity via competitive inhibition of the NS5B protein. Alternatively, a chain-terminator nucleoside analog also may be incorporated into the extending RNA strand. Recently, several patent applications (including WO 99/43691, WO 01/32153, WO 01160315, WO 01179246, WO 01/90121, WO 01/92282, WO 02/48165, WO 02/18404, WO 02/094289, WO 02/057287, WO 02/100415(A2), US 06/040890, WO 02/057425, EP 1674104(A1), EP 1706405(A1), US 06/199783, WO 02/32920, US 04/6784166, WO 05/000864, WO 05/021568) have described nucleoside analogs as anti-HCV agents.

Chikungunya virus (CHIKV) is an insect-borne virus that is transmitted to humans by virus-carrying Aedes Aegypti mosquitoes [Lahariya C, Pradhan S K. Emergence of chikungunya virus in Indian subcontinent after 32 years: a review. J Vect Borne Dis. 2006; 43(4):151-60]. Chikungunya virus (CHIKV) is a member of the genus Alphavirus, in the family Togaviridae. CHIKV was first isolated from the blood of a febrile patient in Tanzania in 1953, and has since been identified repeatedly in west, central and southern Africa and many areas of Asia, and has been cited as the cause of numerous human epidemics in those areas since that time. There have been recent breakouts of CHIKV associated with severe illness. CHIKV causes an illness with symptoms similar to dengue fever. CHIKV manifests itself with an acute febrile phase of the illness lasting only two to five days, followed by a prolonged phase which may include arthralgic disease (joint pain) that affects the joints of the extremities, myalgia (muscular pain), headache, fatigue (weakness), nausea, vomiting and rash. The pain associated with CHIKV infection of the joints persists for weeks or months, or in some cases years. The incubation period (time from infection to illness) can be 2-12 days, but is usually 3-7 days. Acute chikungunya fever typically lasts a few days to a couple of weeks, but some patients have prolonged fatigue lasting several weeks. Additionally, some patients have reported incapacitating joint pain, or arthritis which may last for weeks or months. No deaths, neuro-invasive cases, or hemorrhagic cases related to CHIKV infection have been conclusively documented in the scientific literature. There are currently no specific treatments for Chikungunya virus infection, nor are there any approved vaccines for prevention of infection.

Norovirus is one of four viral genera found in the non-enveloped positive strand RNA family Caliciviridae. The other three species in Caliciviridae are Lagovirus, Vesivirus, and Sapovirus. Sapovirus is the only member of the genus other than Norovirus which utilizes humans as hosts. The Norovirus genome is approximately 7.56 kb with three open reading frames (ORFs). The first ORF codes for nonstructural proteins, including a helicase, a protease, and an RNA-directed RNA polymerase (RDRP), all of which are required for replication of the virus. The remaining two ORFs code for Capsid proteins (Jiang, X. (1993) Virology 195(1):51-61). The numerous strains of Norovirus have been classified into 5 genogroups of which I, IV, and V infect humans (Zheng, D. P., et al. (2006) Virology 346(2):312-323) and are estimated by the CDC to cause approximately 23 million gastroenteritis cases, corresponding to 40% of food-borne illness each year in the US (Mead P. S. (1999) Emerg. Infect. Dis. 5(5):607-625).

Common symptoms are vomiting, diarrhea, and intestinal cramps. Vomiting is the most common symptom in children, while diarrhea is more common in infected adults. Dehydration is a significant concern. The loss of life due to this virus is about 300 patients per year in the United States, and these deaths are usually among patients with a weak immune system (Centers for Disease Control and Prevention. “Norwalk-like viruses:” public health consequences and outbreak management. MMWR 2001; 50 (No. RR-9):3). The incubation period from exposure to full infection is typically 24 to 48 hrs with approximately 30% of infected individuals showing no symptoms. Symptoms generally persist for 24 to 60 hrs (Adler, J. L. and Zickl, R., J. (1969) Infect. Dis. 119:668-673). Viral shedding may last for up to 2 weeks following the infection, however, it is not clear whether this virus is infectious.

Norovirus is transmitted primarily by the fecal-oral route through contaminated food or water, person to person contact, aerosols of vomit or stool samples. Viral titers in stool samples can reach 10⁶ to 10⁷ particles per mL, and particles are stable to temperatures of 0° C. (32° F.) to 60° C. (140° F.) (Duizer, E. et al., (2004) Appl. Environ. Microbiol. 70(8); 4538-4543). The virus is highly infectious, and various sources suggest infection may require inoculation of as few as 10 to 100 viral particles (Centers for Disease Control and Prevention. “Norwalk-like viruses:” public health consequences and outbreak management. MMR 2001; 50 (No. RR-9):3-6). This leads to epidemics in schools, nursing homes, cruise ships, hospitals, or other locations where people congregate.

Norovirus is named for Norwalk-like viruses, a name derived from an outbreak at a school in Norwalk, Ohio in 1968. The viral particle responsible for the Norwalk illness was identified in 1972 by immune electron microscopy following passage of rectal swab filtrates through three sets of human volunteers (Kapikian, A. Z. et al. (1972) J. Virol. 10:1075-1081). In following years, the virus was called small round structured virus due to its electron microscopic image, calicivirus since it a member of the Caliciviridae family, and/or probably most commonly Norwalk-like virus after the originally isolated strain. Common names for the virus include winter vomiting virus, stomach flu, food poisoning, and viral gastroenteritis. While the outcome of infection is generally non-life threatening, the cost of loss of use of facilities and loss of productivity is great, and, consequently, a therapy for treatment of Norovirus infection in humans would be very desirable.

There is currently no approved pharmaceutical treatment for Norovirus infection (http://www.cdc.gov/ncidod/dvrd/revb/gastro/norovirus-qa.htm), and this has probably at least in part been due to the lack of availability of a cell culture system. Recently, a replicon system has been developed for the original Norwalk G-I strain (Chang, K. O., et al. (2006) Virology 353:463-473). Both Norovirus replicons and Hepatitis C replicons require viral helicase, protease, and polymerase to be functional in order for replication of the replicon to occur. Most recently, an in vitro cell culture infectivity assay has been reported utilizing Norovirus genogroup I and II inoculums (Straub, T. M. et al. (2007) Emerg. Infect. Dis. 13(3):396-403). This assay is performed in a rotating-wall bioreactor utilizing small intestinal epithelial cells on microcarrier beads, and at least initially seems as though it would be difficult to screen a meaningful number of compounds with this system. Eventually the infectivity assay may be useful for screening entry inhibitors. Other groups, such as Ligocyte Pharmaceuticals, Inc. (http://www.ligocyte.com/) have focused on trying to develop a vaccine against Noroviruses, however, these efforts have not yet been successful and may prove difficult as has often been the case in viral systems where low replicase fidelity is an evolutionary benefit.

West Nile Virus (WNV) is from the family Flaviviridae and predominantly a mosquito-borne disease. It was first discovered in the West Nile District of Uganda in 1937. According to the reports from the Centers for Disease Control and Prevention, WNV has been found in Africa, the Middle East, Europe, Oceania, west and central Asia, and North America. Its first emergence in North America began in the New York City metropolitan area in 1999. It is a seasonal epidemic in North America that normally erupts in the summer and continues into the fall, presenting a threat to environmental health. Its natural cycle is bird-mosquito-bird and mammal. Mosquitoes, in particular the species Culex pipiens, become infected when they feed on infected birds. Infected mosquitoes then spread WNV to other birds and mammals including humans when they bite. In humans and horses, fatal Encephalitis is the most serious manifestation of WNV infection. WNV can also cause mortality in some infected birds. There is no specific treatment for WNV infection. In cases with milder symptoms, people experience symptoms such as fever and aches that pass on their own, although even healthy people have become sick for several weeks. In more severe cases, people usually need to go to the hospital where they can receive supportive treatment.

Dengue infection is also from the family Flaviviridae and is the most important arthropod-borne infection in Singapore (Epidemiol News Bull 2006, 32, 62-6). Globally, there are an estimated 50 to 100 million cases of dengue fever (DF) and several hundred thousand cases of dengue hemorrhagic fever (DHF) per year with and average fatality fate of 5%. Many patients recover from dengue infection with minimal or no residual illness. Dengue infections are usually asymptomatic, but can present with classic dengue fever, dengue hemorrhagic fever or dengue shock syndrome. Even for outpatients, the need for maintaining adequate hydration is highly important. Dengue infections can be effectively managed by intravenous fluid replacement therapy, and if diagnosed early, fatality rates can be kept below 1%. To manage the pain and fever, patients suspected of having a dengue infection should be given acetaminophen preparations. Aspirin and non-steroidal anti-inflammatory medications may aggravate the bleeding tendency associated with some dengue infection. However, some manifestations of dengue infection previously described include liver failure (Dig Dis Sci 2005, 50, 1146-7), encephalopathy (J Trop Med Public Health 1987, 18, 398-406), and Guillain-Barré syndrome (Intern Med 2006, 45, 563-4).

It has been discovered that, upon incubation in cell culture, or administration in vivo, that 2,6-diamino 2′-C-Me purine nucleosides are converted to the corresponding 6-hydroxy-2,6-diamino 2′-C-Me purine nucleosides. We have also found this to be true for a variety of other 6-substituted purine nucleosides. These compounds act as prodrugs for G or I analogs, much as is the case for the prodrug Abacavir and its in vivo conversion to the corresponding G analog Carbovir ((−)-carbocyclic 2′,3′-didehydro-2′,3′-dideoxyguanosine). This conversion seriously limits the variety of 6-substituted purine nucleosides triphosphates which can be formed in vivo as potential antiviral agents.

In light of the fact that HCV, Norovirus, Sapovirus, Dengue virus, Chikungunya virus and Yellow fever have reached alarming levels worldwide, and have significant and in some cases tragic effects on the effected patient, there remains a strong need to provide new effective pharmaceutical agents to treat these diseases, with agents that have low toxicity to the host.

It would be advantageous to provide new antiviral or chemotherapy agents, compositions including these agents, and methods of treatment using these agents, particularly to treat drug resistant mutant viruses. The present invention provides such agents, compositions and methods.

### SUMMARY OF THE INVENTION

The present invention provides compounds, methods and compositions for treating or preventing HCV, Norovirus, Sapovirus, Dengue virus, Chikungunya virus or Yellow fever infection in a host. The methods involve administering a therapeutically or prophylactically-effective amount of at least one compound as described herein to treat or prevent an infection by, or an amount sufficient to reduce the biological activity of, HCV, Norovirus, Sapovirus, Dengue virus, Chikungunya virus or Yellow fever infection. The pharmaceutical compositions include one or more of the compounds described herein, in combination with a pharmaceutically acceptable carrier or excipient, for treating a host with cancer or infected with HCV, Norovirus, Sapovirus, Dengue virus, Chikungunya virus or Yellow fever. The formulations can further include at least one further therapeutic agent. In addition, the present invention includes processes for preparing such compounds.

As with Hepatitis C replicons, Norovirus replicons require viral helicase, protease, and polymerase to be functional in order for replication of the replicon to occur. The replicons can be used in high throughput assays, which evaluate whether a compound to be screened for activity inhibits the ability of Norovirus helicase, protease, and/or polymerase to function, as evidenced by an inhibition of replication of the replicon.

The compounds are monophosphate forms of various 2,6-diamino 2′-C-methyl purine nucleosides, or analogs of the monophosphate forms, which also become triphosphorylated when administered in vivo. We have discovered, quite surprisingly, that preparation of the monophosphate prodrug of these nucleosides partially (or potentially fully) protects the 6-amino group from conversion to the G analog. By preparing the monophosphate prodrugs, we have developed a method for delivering nucleotide triphosphates to the polymerase, which before this invention was not possible, or at least not possible at therapeutically-relevant concentrations. This invention, in some embodiments, delivers two triphosphates to the polymerase one of which is recognized as a G analog and the other is recognized as an A analog. This invention allows for a new and novel series of nucleotide triphosphates (along with mixtures with the corresponding G analog) to be prepared in vivo and enlisted as antiviral agents.

The compounds described herein include monophosphate analogs of β-D-2,6-diamino 2-C-methyl purine nucleosides. In one embodiment, the active compound is of formula (A); in another embodiment, the active compound is of formula (B):

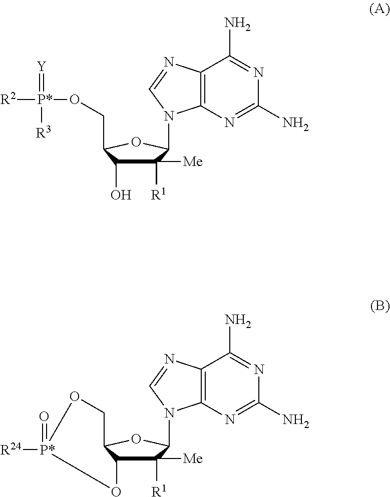

or a pharmaceutically acceptable salt or prodrug thereof, wherein:
when chirality exists at the phosphorous center it may be wholly or partially Rₚ or Sₚ or any mixture thereof.
R¹ is OH or F;
Y is O or S;
R²⁴ is selected from OR¹⁵,

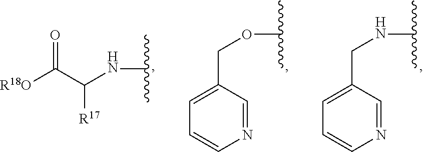

and
fatty alcohol derived (for example but not limited to:

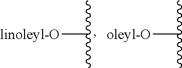

(wherein R¹⁵, R¹⁷, and R¹⁸ are as defined below);
R² and R³, when administered in vivo, are capable of providing the nucleoside monophosphate or thiomonophosphate that is either partially or fully resistant to 6-NH₂ deamination in a biological system. Representative R² and R³ are independently selected from:
   (a) OR¹⁵ where R¹⁵ is selected from H, Li, Na, K, phenyl and pyridinyl; Phenyl and pyridinyl are substituted with one to three substituents independently selected from the group consisting of (CH₂)₀₋₆CO₂R¹⁶ and (CH₂)₀₋₆CON(R¹⁶)₂;
      R¹⁶ is independently H, C₁₋₂₀ alkyl, the carbon chain derived from a fatty alcohol (such as oleyl alcohol, octacosanol, triacontanol, linoleyl alcohol, and etc) or C₁₋₂₀ alkyl substituted with a lower alkyl, alkoxy, di(lower alkyl)-amino, fluoro, C₃₋₁₀ cycloalkyl, cycloalkyl alkyl, cycloheteroalkyl, aryl, such as phenyl, heteroaryl, such as, pyridinyl, substituted aryl, or substituted heteroaryl; wherein the substituents are C₁₋₅ alkyl, or C₁₋₅ alkyl substituted with a lower alkyl, alkoxy, di(lower alkyl)-amino, fluoro, C₃₋₁₀ cycloalkyl, or cycloalkyl;

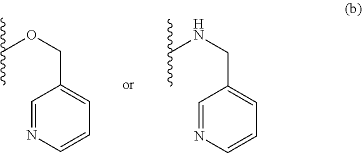 [SMILES: *OCc1cccnc1 ; *NCc1cccnc1]

(c) the ester of an L-amino acid

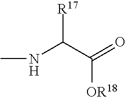

where R¹⁷ is restricted to those occurring in natural L-amino acids, and R¹⁸ is H, C₁₋₂₀ alkyl, the carbon chain derived from a fatty alcohol (such as oleyl alcohol, octacosanol, triacontanol, linoleyl alcohol, and etc) or C₁₋₂₀ alkyl substituted with a lower alkyl, alkoxy, di(lower alkyl)-amino, fluoro, C₃₋₁₀ cycloalkyl, cycloalkyl alkyl, cycloheteroalkyl, aryl, such as phenyl, heteroaryl, such as, pyridinyl, substituted aryl, or substituted heteroaryl; wherein the substituents are C₁₋₅ alkyl, or C₁₋₅ alkyl substituted with a lower alkyl, alkoxy, di(lower alkyl)-amino, fluoro, C₃₋₁₀ cycloalkyl, or cycloalkyl;
(d) R² and R³ can come together to form a ring

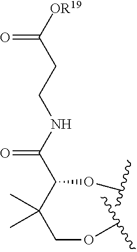

where R¹⁹ is H, C₁₋₂₀ alkyl, C₁₋₂₀ alkenyl, the carbon chain derived from a fatty alcohol (such as oleyl alcohol, octacosanol, triacontanol, linoleyl alcohol, etc) or C₁₋₂₀ alkyl substituted with a lower alkyl, alkoxy, di(lower alkyl)-amino, fluoro, C₃₋₁₀ cycloalkyl, cycloalkyl alkyl, cycloheteroalkyl, aryl, such as phenyl, heteroaryl, such as, pyridinyl, substituted aryl, or substituted heteroaryl; wherein the substituents are C₁₋₅ alkyl, or C₁₋₅ alkyl substituted with a lower alkyl, alkoxy, di(lower alkyl)-amino, fluoro, C₃₋₁₀ cycloalkyl, or cycloalkyl;
(e) R² and R³ can come together to form a ring selected

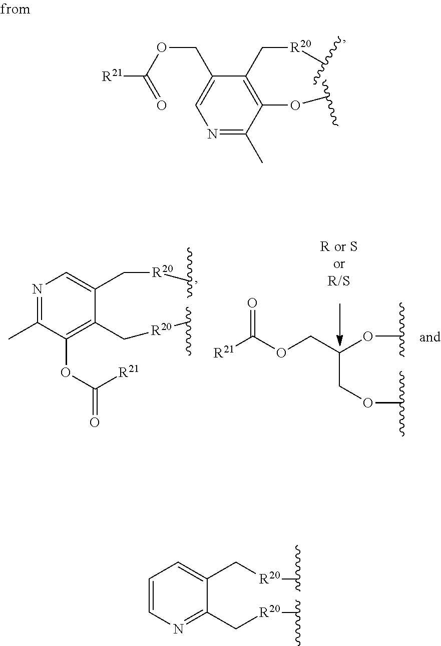

where R²⁰ is O or NH and
R²¹ is selected from H, C₁₋₂₀ alkyl, C₁₋₂₀ alkenyl, the carbon chain derived from a fatty acid (such as oleic acid, linoleic acid, and the like), and C₁₋₂₀ alkyl substituted with a lower alkyl, alkoxy, di(lower alkyl)-amino, fluoro, C₃₋₁₀ cycloalkyl, cycloalkyl alkyl, cycloheteroalkyl, aryl, such as phenyl, heteroaryl, such as pyridinyl, substituted aryl, or substituted heteroaryl; wherein the substituents are C₁₋₅ alkyl, or C₁₋₅ alkyl substituted with a lower alkyl, alkoxy, di(lower alkyl)-amino, fluoro, C₃₋₁₀ cycloalkyl, or cycloalkyl.

The compounds can be prepared, for example, by preparing the 5′-OH analogs, then converting these to the mono-phosphate analogs.

In addition, the compounds described herein are inhibitors of HCV, Norovirus, Sapovirus, Dengue virus, Chikungunya virus and/or Yellow fever. Therefore, these compounds can also be used to treat patients that are co-infected with HCV, Norovirus, Sapovirus, Dengue virus, Chikungunya virus and/or Yellow fever.

### BRIEF DESCRIPTION OF THE FIGURES

FIG. 1: ORTEP drawing of 24

FIG. 2: ORTEP drawing of 25 (S_{P})

FIG. 3: ORTEP drawing of 25 (S_{P})

FIG. 4: Incorporation of ((2R,3S,4R,5R)-5-(2,6-diamino-9H-purin-9-yl)-3,4-dihydroxytetrahydrofuran-2-yl)methyl tetrahydrogen triphosphate by HCV NS5B.

FIG. 5: Incorporation of ((2R,3S,4R,5R)-5-(2-amino-6-hydroxy-9H-purin-9-yl)-3,4-dihydroxytetrahydrofuran-2-yl)methyl tetrahydrogen triphosphateby HCV NS5B.

FIG. 6: LC/MS analysis of nucleotides formed after 4 hr incubation in Huh7 cells of 50 μM 12.

FIG. 7: LC/MS analysis of nucleotides formed after 4 hr incubation in Huh7 cells of 50 μM 8a.

FIG. 8: Metabolic suppression with 8a gives intracellular delivery of both a 2,6-diamino and a G triphosphate

FIG. 9: LC/MS analysis of nucleotides formed after 4 hr incubation in Huh7 cells of 50 μM 8b-up.

FIG. 10: Metabolic suppression with 8b-up gives intracellular delivery of both a 2,6-diamino and a G triphosphate.

FIG. 11: Intracellular metabolism of DAPD in PBM cells at a concentration of 50 μM, over a 4 h period, at 37° C.

FIG. 12: Incubation of phosphoramidate RS-864, which contains a 6-amino group and a 5′-MP prodrug, in PBM cells at a concentration of 50 μM, over a 4 h period, at 37° C.

### DETAILED DESCRIPTION

The 2,6-diamino-2′-C-Me purine nucleosides monophosphate prodrugs described herein show inhibitory activity against HCV, Norovirus, Saporovirus, Dengue virus, Chikungunya virus and Yellow fever. Therefore, the compounds can be used to treat or prevent a viral infection in a host, or reduce the biological activity of the virus. The host can be a mammal, and in particular, a human, infected with HCV, Norovirus, Saporovirus, Dengue virus, Chikungunya virus and/or Yellow fever. The methods involve administering an effective amount of one or more of the 2,6-diamino 2′-C-Me purine nucleotides monophosphate prodrugs described herein.

Pharmaceutical formulations including one or more compounds described herein, in combination with a pharmaceutically acceptable carrier or excipient, are also disclosed. In one embodiment, the formulations include at least one compound described herein and at least one further therapeutic agent.

The present invention will be better understood with reference to the following definitions:

### I. Definitions

The term “independently” is used herein to indicate that the variable, which is independently applied, varies independently from application to application. Thus, in a compound such as R″XYR″, wherein R″ is “independently carbon or nitrogen,” both R″ can be carbon, both R″ can be nitrogen, or one R″ can be carbon and the other R″ nitrogen.

As used herein, the term “enantiomerically pure” refers to a nucleotide composition that comprises at least approximately 95%, and, preferably, approximately 97%, 98%, 99% or 100% of a single enantiomer of that nucleotide.

As used herein, the term “substantially free of” or “substantially in the absence of” refers to a nucleotide composition that includes at least 85 to 90% by weight, preferably 95% to 98% by weight, and, even more preferably, 99% to 100% by weight, of the designated enantiomer of that nucleotide. In a preferred embodiment, the compounds described herein are substantially free of enantiomers.

Similarly, the term “isolated” refers to a nucleotide composition that includes at least 85 to 90% by weight, preferably 95% to 98% by weight, and, even more preferably, 99% to 100% by weight, of the nucleotide, the remainder comprising other chemical species or enantiomers.

In some cases the phosphorus atom may be chiral herein termed “P*” or “P” which means that and that it has a designation of “R” or “S” corresponding to the accepted meanings of Cahn-Ingold-Prelog rules for such assignment. Prodrugs of Formulaa A and B may exist as a mixture of diastereomers due to the chirality at the phosphorus center. When chirality exists at the phosphorous center it may be wholly or partially Rₚ or Sₚ or any mixture thereof.

The term “alkyl,” as used herein, unless otherwise specified, refers to a saturated straight, branched, or cyclic, primary, secondary, or tertiary hydrocarbons, including both substituted and unsubstituted alkyl groups. The alkyl group can be optionally substituted with any moiety that does not otherwise interfere with the reaction or that provides an improvement in the process, including but not limited to but limited to halo, haloalkyl, hydroxyl, carboxyl, acyl, aryl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, either unprotected, or protected as necessary, as known to those skilled in the art, for example, as taught in Greene, et al., Protective Groups in Organic Synthesis, John Wiley and Sons, Second Edition, 1991, hereby incorporated by reference. Specifically included are CF₃ and CH₂CF₃

In the text, whenever the term C (alkyl range) is used, the term independently includes each member of that class as if specifically and separately set out. The term “alkyl” includes C₁₋₂₂ alkyl moieties, and the term “lower alkyl” includes C₁₋₆ alkyl moieties. It is understood to those of ordinary skill in the art that the relevant alkyl radical is named by replacing the suffix “-ane” with the suffix “-yl”.

The term “alkenyl” refers to an unsaturated, hydrocarbon radical, linear or branched, in so much as it contains one or more double bonds. The alkenyl group disclosed herein can be optionally substituted with any moiety that does not adversely affect the reaction process, including but not limited to but not limited to those described for substituents on alkyl moieties. Non-limiting examples of alkenyl groups include ethylene, methylethylene, isopropylidene, 1,2-ethane-diyl, 1,1-ethane-diyl, 1,3-propane-diyl, 1,2-propane-diyl, 1,3-butane-diyl, and 1,4-butane-diyl.

The term “alkynyl” refers to an unsaturated, acyclic hydrocarbon radical, linear or branched, in so much as it contains one or more triple bonds. The alkynyl group can be optionally substituted with any moiety that does not adversely affect the reaction process, including but not limited to those described above for alkyl moeities. Non-limiting examples of suitable alkynyl groups include ethynyl, propynyl, hydroxypropynyl, butyn-1-yl, butyn-2-yl, pentyn-1-yl, pentyn-2-yl, 4-methoxypentyn-2-yl, 3-methylbutyn-1-yl, hexyn-1-yl, hexyn-2-yl, and hexyn-3-yl, 3,3-dimethylbutyn-1-yl radicals.

The term “alkylamino” or “arylamino” refers to an amino group that has one or two alkyl or aryl substituents, respectively.

The term “protected” as used herein and unless otherwise defined refers to a group that is added to an oxygen, nitrogen, or phosphorus atom to prevent its further reaction or for other purposes. A wide variety of oxygen and nitrogen protecting groups are known to those skilled in the art of organic synthesis, and are described, for example, in Greene et al., Protective Groups in Organic Synthesis, supra.

The term “aryl”, alone or in combination, means a carbocyclic aromatic system containing one, two or three rings wherein such rings can be attached together in a pendent manner or can be fused. Non-limiting examples of aryl include phenyl, biphenyl, or naphthyl, or other aromatic groups that remain after the removal of a hydrogen from an aromatic ring. The term aryl includes both substituted and unsubstituted moieties. The aryl group can be optionally substituted with any moiety that does not adversely affect the process, including but not limited to but not limited to those described above for alkyl moieties. Non-limiting examples of substituted aryl include heteroarylamino, N-aryl-N-alkylamino, N-heteroarylamino-N-alkylamino, heteroaralkoxy, arylamino, aralkylamino, arylthio, monoarylamidosulfonyl, arylsulfonamido, diarylamidosulfonyl, monoaryl amidosulfonyl, arylsulfinyl, arylsulfonyl, heteroarylthio, heteroarylsulfinyl, heteroarylsulfonyl, aroyl, heteroaroyl, aralkanoyl, heteroaralkanoyl, hydroxyaralkyl, hydroxyheteroaralkyl, haloalkoxyalkyl, aryl, aralkyl, aryloxy, aralkoxy, aryloxyalkyl, saturated heterocyclyl, partially saturated heterocyclyl, heteroaryl, heteroaryloxy, heteroaryloxyalkyl, arylalkyl, heteroarylalkyl, arylalkenyl, and heteroarylalkenyl, carboaralkoxy.

The terms “alkaryl” or “alkylaryl” refer to an alkyl group with an aryl substituent. The terms “aralkyl” or “arylalkyl” refer to an aryl group with an alkyl substituent.

The term “halo,” as used herein, includes chloro, bromo, iodo and fluoro.

The term “acyl” refers to a carboxylic acid ester in which the non-carbonyl moiety of the ester group is selected from straight, branched, or cyclic alkyl or lower alkyl, alkoxyalkyl including but not limited to methoxymethyl, aralkyl including but not limited to benzyl, aryloxyalkyl such as phenoxymethyl, aryl including but not limited to phenyl optionally substituted with halogen (F, Cl, Br, I), alkyl (including but not limited to C₁, C₂, C₃, and C₄) or alkoxy (including but not limited to C₁, C₂, C₃, and C₄), sulfonate esters such as alkyl or aralkyl sulphonyl including but not limited to methanesulfonyl, the mono, di or triphosphate ester, trityl or monomethoxytrityl, substituted benzyl, trialkylsilyl (e.g., dimethyl-t-butylsilyl) or diphenylmethylsilyl. Aryl groups in the esters optimally comprise a phenyl group. The term “lower acyl” refers to an acyl group in which the non-carbonyl moiety is lower alkyl.

The terms “alkoxy” and “alkoxyalkyl” embrace linear or branched oxy-containing radicals having alkyl moieties, such as methoxy radical. The term “alkoxyalkyl” also embraces alkyl radicals having one or more alkoxy radicals attached to the alkyl radical, that is, to form monoalkoxyalkyl and dialkoxyalkyl radicals. The “alkoxy” radicals can be further substituted with one or more halo atoms, such as fluoro, chloro or bromo, to provide “haloalkoxy” radicals. Examples of such radicals include fluoromethoxy, chloromethoxy, trifluoromethoxy, difluoromethoxy, trifluoroethoxy, fluoroethoxy, tetrafluoroethoxy, pentafluoroethoxy, and fluoropropoxy.

The term “alkylamino” denotes “monoalkylamino” and “dialkylamino” containing one or two alkyl radicals, respectively, attached to an amino radical. The terms arylamino denotes “monoarylamino” and “diarylamino” containing one or two aryl radicals, respectively, attached to an amino radical. The term “aralkylamino”, embraces aralkyl radicals attached to an amino radical. The term aralkylamino denotes “monoaralkylamino” and “diaralkylamino” containing one or two aralkyl radicals, respectively, attached to an amino radical. The term aralkylamino further denotes “monoaralkyl monoalkylamino” containing one aralkyl radical and one alkyl radical attached to an amino radical.

The term “heteroatom,” as used herein, refers to oxygen, sulfur, nitrogen and phosphorus.

The terms “heteroaryl” or “heteroaromatic,” as used herein, refer to an aromatic that includes at least one sulfur, oxygen, nitrogen or phosphorus in the aromatic ring.

The term “heterocyclic,” “heterocyclyl,” and cycloheteroalkyl refer to a nonaromatic cyclic group wherein there is at least one heteroatom, such as oxygen, sulfur, nitrogen, or phosphorus in the ring.

Nonlimiting examples of heteroaryl and heterocyclic groups include furyl, furanyl, pyridyl, pyrimidyl, thienyl, isothiazolyl, imidazolyl, tetrazolyl, pyrazinyl, benzofuranyl, benzothiophenyl, quinolyl, isoquinolyl, benzothienyl, isobenzofuryl, pyrazolyl, indolyl, isoindolyl, benzimidazolyl, purinyl, carbazolyl, oxazolyl, thiazolyl, isothiazolyl, 1,2,4-thiadiazolyl, isooxazolyl, pyrrolyl, quinazolinyl, cinnolinyl, phthalazinyl, xanthinyl, hypoxanthinyl, thiophene, furan, pyrrole, isopyrrole, pyrazole, imidazole, 1,2,3-triazole, 1,2,4-triazole, oxazole, isoxazole, thiazole, isothiazole, pyrimidine or pyridazine, and pteridinyl, aziridines, thiazole, isothiazole, 1,2,3-oxadiazole, thiazine, pyridine, pyrazine, piperazine, pyrrolidine, oxaziranes, phenazine, phenothiazine, morpholinyl, pyrazolyl, pyridazinyl, pyrazinyl, quinoxalinyl, xanthinyl, hypoxanthinyl, pteridinyl, 5-azacytidinyl, 5-azauracilyl, triazolopyridinyl, imidazolopyridinyl, pyrrolopyrimidinyl, pyrazolopyrimidinyl, adenine, N⁶-alkylpurines, N⁶-benzylpurine, N⁶-halopurine, N⁶-vinypurine, N⁶-acetylenic purine, N⁶-acyl purine, N⁶-hydroxyalkyl purine, N⁶-thioalkyl purine, thymine, cytosine, 6-azapyrimidine, 2-mercaptopyrmidine, uracil, N⁵-alkylpyrimidines, N⁵-benzylpyrimidines, N⁵-halopyrimidines, N⁵-vinylpyrimidine, N⁵-acetylenic pyrimidine, N⁵-acyl pyrimidine, N⁵-hydroxyalkyl purine, and N⁶-thioalkyl purine, and isoxazolyl. The heteroaromatic group can be optionally substituted as described above for aryl. The heterocyclic or heteroaromatic group can be optionally substituted with one or more substituents selected from halogen, haloalkyl, alkyl, alkoxy, hydroxy, carboxyl derivatives, amido, amino, alkylamino, and dialkylamino. The heteroaromatic can be partially or totally hydrogenated as desired. As a nonlimiting example, dihydropyridine can be used in place of pyridine. Functional oxygen and nitrogen groups on the heterocyclic or heteroaryl group can be protected as necessary or desired. Suitable protecting groups are well known to those skilled in the art, and include trimethylsilyl, dimethylhexylsilyl, t-butyldimethylsilyl, and t-butyldiphenylsilyl, trityl or substituted trityl, alkyl groups, acyl groups such as acetyl and propionyl, methanesulfonyl, and p-toluenelsulfonyl. The heterocyclic or heteroaromatic group can be substituted with any moiety that does not adversely affect the reaction, including but not limited to but not limited to those described above for aryl.

The term “host,” as used herein, refers to a unicellular or multicellular organism in which the virus can replicate, including but not limited to cell lines and animals, and, preferably, humans. Alternatively, the host can be carrying a part of the viral genome, whose replication or function can be altered by the compounds of the present invention. The term host specifically refers to infected cells, cells transfected with all or part of the viral genome and animals, in particular, primates (including but not limited to chimpanzees) and humans. In most animal applications of the present invention, the host is a human patient. Veterinary applications, in certain indications, however, are clearly contemplated by the present invention (such as for use in treating chimpanzees).

The term “peptide” refers to various natural or synthetic compounds containing two to one hundred amino acids linked by the carboxyl group of one amino acid to the amino group of another.

The term “pharmaceutically acceptable salt or prodrug” is used throughout the specification to describe any pharmaceutically acceptable form (such as an ester, phosphate ester, salt of an ester or a related group) of a nucleotide compound which, upon administration to a patient, provides the nucleotide monophosphate compound. Pharmaceutically acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art. Pharmaceutically acceptable prodrugs refer to a compound that is metabolized, for example hydrolyzed or oxidized, in the host to form the compound of the present invention. Typical examples of prodrugs include compounds that have biologically labile protecting groups on functional moieties of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. The prodrug forms of the compounds of this invention can possess antiviral activity, can be metabolized to form a compound that exhibits such activity, or both.

Prodrugs also include amino acid esters of the disclosed nucleosides (see, e.g., European Patent Specification No. 99493, the text of which is incorporated by reference, which describes amino acid esters of acyclovir, specifically the glycine and alanine esters which show improved water-solubility compared with acyclovir itself, and U.S. Pat. No. 4,957,924 (Beauchamp), which discloses the valine ester of acyclovir, characterized by side-chain branching adjacent to the α-carbon atom, which showed improved bioavailability after oral administration compared with the alanine and glycine esters). A process for preparing such amino acid esters is disclosed in U.S. Pat. No. 4,957,924 (Beauchamp), the text of which is incorporated by reference. As an alternative to the use of valine itself, a functional equivalent of the amino acid can be used (e.g., an acid halide such as the acid chloride, or an acid anhydride). In such a case, to avoid undesirable side-reactions, it may be advantageous to use an amino-protected derivative.

### II. Active Compound

In one embodiment, the compounds have the formula provided below:

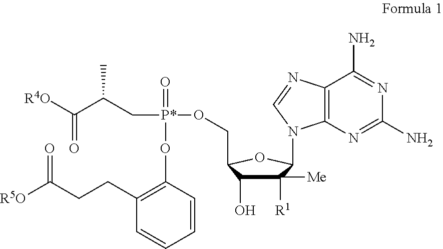

Where R¹ is OH, or F, and R⁴ and R⁵ are, independently, C₁₋₆ alkyl, or a carbon chain derived from a fatty alcohol. Carbon chains derived from fatty alcohols typically have between 8 and 34 carbon atoms, and can include 0, 1, or more double bonds. Fatty alcohols are often but not always obtained by reduction of the corresponding fatty acid. The term “fatty acid radical” is used herein to refer to these carbon chains which still contain the carbonyl group of the acid as the attachment point. For example, oleyl alcohol is cis-9-octadecen-1-ol, an 18 carbon chain with a single double bond. The carbon chain derived from oleyl alcohol (also referred to herein as “carbon chain of oleyl”) is cis-9-octadecene. Representative values for R¹, R⁴, and R⁵ are provided below:

| R¹ | R⁴ | R⁵ |
|---|---|---|
| OH | Me | Me |
| F | Me | Me |
| OH | Et | Et |
| F | Et | Et |
| OH | i-Pr | i-Pr |
| F | i-Pr | i-Pr |
| OH | carbon | carbon |
| | chain of | chain |
| | oleyl | of oleyl |
| F | carbon | carbon |
| | chain of | chain |
| | oleyl | of oleyl |

In another embodiment, the compounds have the following formula:

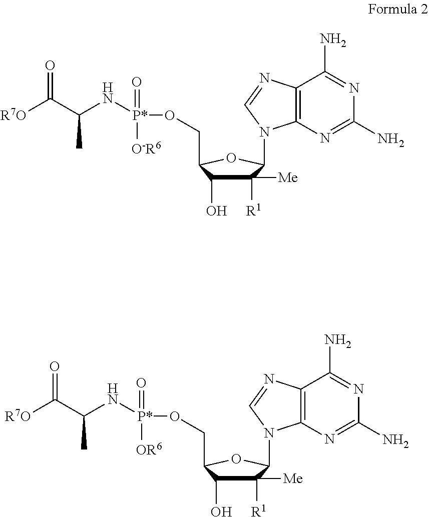

wherein R¹ is as defined in Claim 1, R⁶ is a alkali metal or H, and R⁷ is a carbon chain derived from a fatty alcohol. Representative values for R¹, R⁶, and R⁷ are provided below:

| | | R⁷ (carbon |
|---|---|---|
| R¹ | R⁶ | chain of) |
| OH | Na | linoleyl |
| F | Na | linoleyl |
| OH | K | linoleyl |
| F | K | linoleyl |
| OH | Na | oleyl |
| F | Na | oleyl |
| OH | K | oleyl |
| F | K | oleyl |
| OH | H | linoleyl |
| F | H | linoleyl |
| OH | H | oleyl |
| F | H | oleyl |

In another embodiment, the compounds have the following formula:

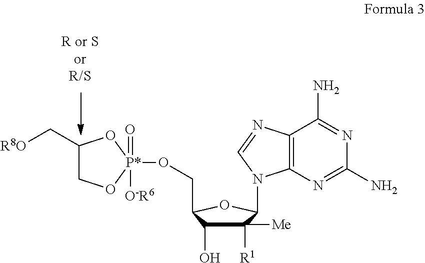

wherein R¹ is as defined in Claim 1, R⁸ is —C(O)—C₈₋₃₄ alkyl or alkenyl, or a fatty acid radical. Representative values for R¹, R⁶, and R⁷ are provided below:

| R¹ | R⁸ |
|---|---|
| OH | Linoleyl acid |
| | radical |
| F | linoleyl acid |
| | radical |
| OH | oleyl acid |
| | radical |
| F | oleyl acid |
| | radical |

In a fourth embodiment, the compounds have the formulas:

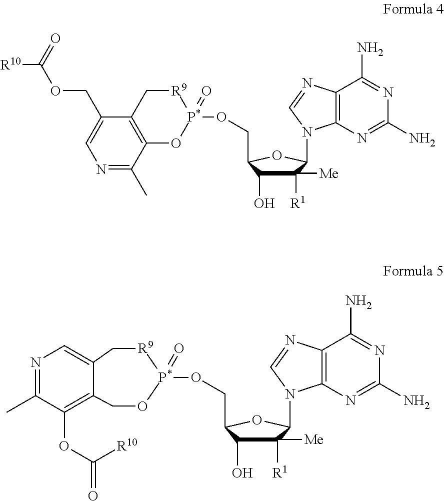

wherein R¹ is as defined in Formula 1, R⁹ is O or NH, and R¹⁰ being C₁₋₆ alkyl or a carbon chain derived from a fatty alcohol. Representative values for R¹, R⁹, and R¹⁰ are provided below:

| R⁷ | R⁹ | R¹⁰ |
|---|---|---|
| OH | O | Me |
| F | O | Me |
| OH | NH | Me |
| F | NH | Me |
| OH | O | Et |
| F | O | Et |
| OH | NH | Et |
| F | NH | Et |
| OH | O | i-Pr |
| F | O | i-Pr |
| OH | NH | i-Pr |
| F | NH | i-Pr |
| OH | O | carbon |
| | | chain of |
| | | oleyl |
| F | O | carbon |
| | | chain of |
| | | oleyl |
| OH | NH | carbon |
| | | chain of |
| | | oleyl |
| F | NH | carbon |
| | | chain of |
| | | oleyl |

In a fifth embodiment, the compounds have one of the following formulas:

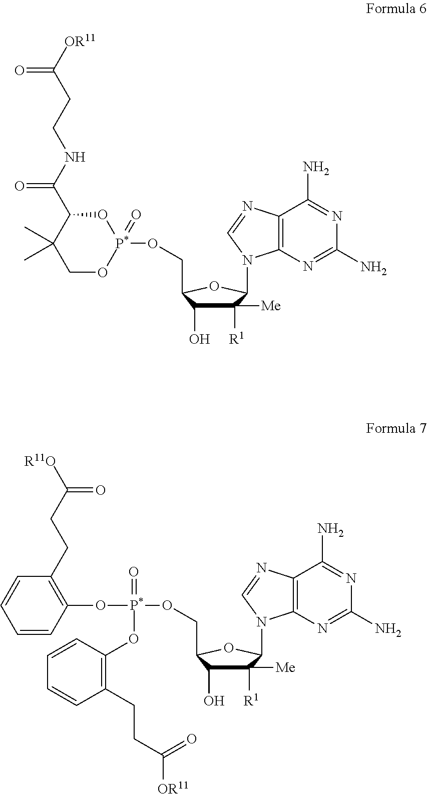

wherein R¹ is as defined in Formula 1, R¹¹ is C₁₋₆ alkyl or a carbon chain derived from a fatty alcohol. Representative values for R¹ and R¹¹ are provided below:

| R¹ | R¹¹ |
|---|---|
| OH | Me |
| F | Me |
| OH | Et |
| F | Et |
| OH | i-Pr |
| F | i-Pr |
| OH | carbon |
| | chain of |
| | oleyl |
| F | carbon |
| | chain of |
| | oleyl |

In a sixth embodiment, the compounds have one of the following formulas:

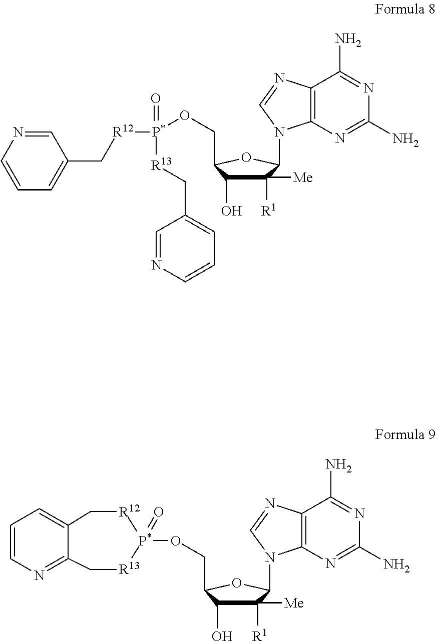

wherein R¹ is as defined in Formula 1, and R¹² and R¹³ are O or NH. Representative values for R¹, R¹², and R¹³ are provided below:

| R¹ | R¹² | R¹³ |
|---|---|---|
| OH | O | O |
| F | O | O |
| OH | O | NH |
| F | O | NH |
| OH | NH | NH |
| F | NH | NH |

In a seventh embodiment, the compounds have the formula:

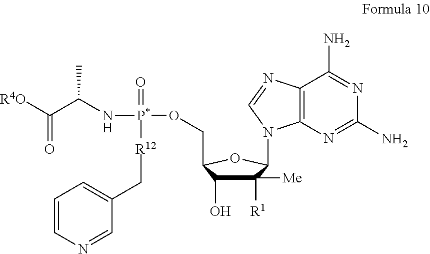

wherein R¹ is as defined in Formula 1, R⁴ is C₁₋₆ alkyl or a carbon chain derived from a fatty alcohol, and R¹² is O or NH. Representative values for R¹, R⁴, and R¹² are provided below:

| R¹ | R⁴ | R¹² |
|---|---|---|
| OH | Me | O |
| F | Me | O |
| OH | Et | O |
| F | Et | O |
| OH | i-Pr | O |
| F | i-Pr | O |
| OH | carbon | O |
| | chain of | |
| | oleyl | |
| OH | Me | NH |
| F | Me | NH |
| OH | Et | NH |
| F | Et | NH |
| OH | i-Pr | NH |
| F | i-Pr | NH |
| OH | carbon | NH |
| | chain of | |
| | oleyl | |
| F | carbon | NH |
| | chain of | |
| | oleyl | |

In an eighth embodiment, the compounds have the following formula:

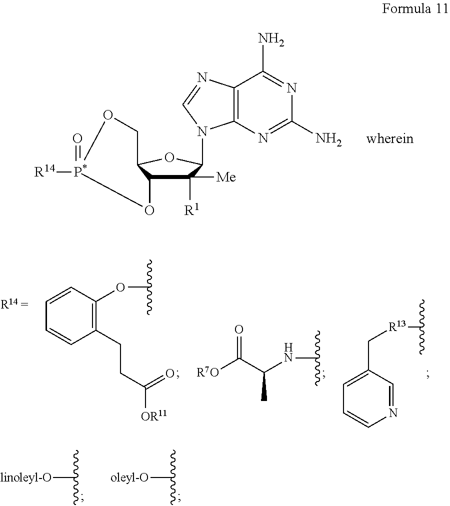

and R¹, R¹¹, R⁷ and R¹³ are as defined above

Processes for making a single or enriched diastereomer at the phosphorous center based on the leaving group of a 4-(substituted sulfonyl)phenol are also disclosed. Wherein the [symbol] represents a group or groups which may be converted to a monophosphate in a biological system containing a fixed chiral center and G¹ is a groups such as methyl, trifluoromethyl, phenyl, and etc. The Rₚ/Sₚ mixture may be separated via chromatography or crystallization. Alternatively, the Rₚ/Sₚ mixture may be separated by reaction with a 4-(substituted thio)phenol in which only one or predominantly only one diastereomer reacts with said 4-(substituted thio)phenol allowing for separation via chromatography or crystallization. Subsequent to separation, oxidation of the thioether to the sulfone allows for use as a monophosphate prodrug-forming reagent.

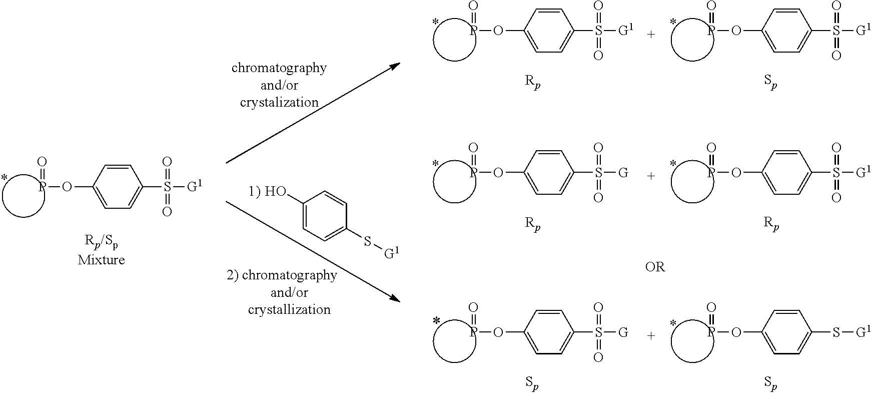

Processes for making a single or enriched diastereomer at the phosphorous center of a nucleoside based on the leaving group of 4-(methylsulfonyl)phenol are also disclosed. The processes involve: a) Reaction of the phenyl phosphorodichloridate, F1, with 4-(methylsulfonyl)phenol followed by ethyl alanine to give G1 as a approximate 1:1 Rₚ/Sₚ mixture; b) Oxidation to the sulfone H1; c) reaction of the H1 Rₚ/Sₚ with a 4-(methylthio)phenol in which only one diastereomer reacts allowing for separation via chromatography c) subsequent to separation the methyl thio J1 is oxidized to the single or enriched diastereomer sulfone I1; d) reaction of the single or enriched diastereomer sulfone I1 with the 5′-OH of a nucleoside allows for the formation of single or enriched diastereomer nucleoside J1; e) reaction of the single or enriched diastereomer sulfone I1 with 4-(methylthio)phenol inverts the phosphorous center forming L1 that contains the opposite phosphorous stereochemistry relative to I1; f) oxidation of L1 to the sulfone and reaction with the 5′-OH of a nucleoside allows for the formation of a single or enriched nucleoside prodrug diastereomer with opposite phosphorous stereochemistry relative to J1.

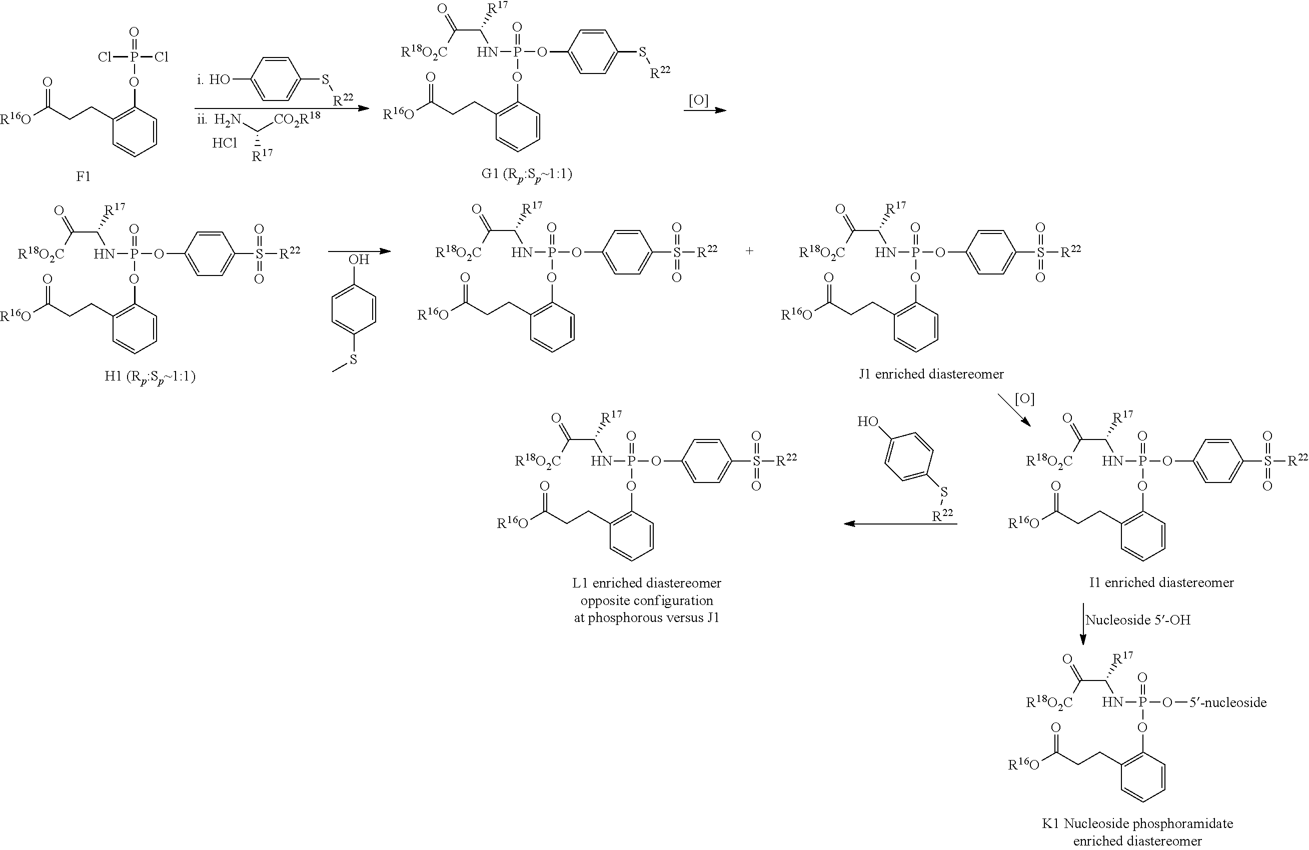

In the above embodiments, in some cases, the phosphorus atom may be chiral herein termed “P*” or “P” which means that and that it has a designation of “R” or “S” corresponding to the accepted meanings of Cahn-Ingold-Prelog rules for such assignment. These embodiments may exist as a mixture of diastereomers due to the chirality at the phosphorus center. When chirality exists at the phosphorous center of these embodiments it may be wholly or partially Rₚ or Sₚ or any mixture thereof.

### III. Stereoisomerism and Polymorphism

The compounds described herein may have asymmetric centers and occur as racemates, racemic mixtures, individual diastereomers or enantiomers, with all isomeric forms being included in the present invention. Compounds of the present invention having a chiral center can exist in and be isolated in optically active and racemic forms. Some compounds can exhibit polymorphism. The present invention encompasses racemic, optically-active, polymorphic, or stereoisomeric forms, or mixtures thereof, of a compound of the invention, which possess the useful properties described herein. The optically active forms can be prepared by, for example, resolution of the racemic form by recrystallization techniques, by synthesis from optically-active starting materials, by chiral synthesis, or by chromatographic separation using a chiral stationary phase or by enzymatic resolution. One can either purify the respective nucleoside, then derivatize the nucleoside to form the compounds described herein, or purify the nucleotides themselves.

Optically active forms of the compounds can be prepared using any method known in the art, including but not limited to by resolution of the racemic form by recrystallization techniques, by synthesis from optically-active starting materials, by chiral synthesis, or by chromatographic separation using a chiral stationary phase.

Examples of methods to obtain optically active materials include at least the following.
i) physical separation of crystals: a technique whereby macroscopic crystals of the individual enantiomers are manually separated. This technique can be used if crystals of the separate enantiomers exist, i.e., the material is a conglomerate, and the crystals are visually distinct;
ii) simultaneous crystallization: a technique whereby the individual enantiomers are separately crystallized from a solution of the racemate, possible only if the latter is a conglomerate in the solid state;
iii) enzymatic resolutions: a technique whereby partial or complete separation of a racemate by virtue of differing rates of reaction for the enantiomers with an enzyme;
iv) enzymatic asymmetric synthesis: a synthetic technique whereby at least one step of the synthesis uses an enzymatic reaction to obtain an enantiomerically pure or enriched synthetic precursor of the desired enantiomer;
v) chemical asymmetric synthesis: a synthetic technique whereby the desired enantiomer is synthesized from an achiral precursor under conditions that produce asymmetry (i.e., chirality) in the product, which can be achieved using chiral catalysts or chiral auxiliaries;
vi) diastereomer separations: a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
vii) first- and second-order asymmetric transformations: a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;
viii) kinetic resolutions: this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;
ix) enantiospecific synthesis from non-racemic precursors: a synthetic technique whereby the desired enantiomer is obtained from non-chiral starting materials and where the stereochemical integrity is not or is only minimally compromised over the course of the synthesis;
x) chiral liquid chromatography: a technique whereby the enantiomers of a racemate are separated in a liquid mobile phase by virtue of their differing interactions with a stationary phase (including but not limited to via chiral HPLC). The stationary phase can be made of chiral material or the mobile phase can contain an additional chiral material to provoke the differing interactions;
xi) chiral gas chromatography: a technique whereby the racemate is volatilized and enantiomers are separated by virtue of their differing interactions in the gaseous mobile phase with a column containing a fixed non-racemic chiral adsorbent phase;
xii) extraction with chiral solvents: a technique whereby the enantiomers are separated by virtue of preferential dissolution of one enantiomer into a particular chiral solvent;
xiii) transport across chiral membranes: a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane that allows only one enantiomer of the racemate to pass through.

Chiral chromatography, including but not limited to simulated moving bed chromatography, is used in one embodiment. A wide variety of chiral stationary phases are commercially available.

### IV. Nucleotide Salt or Prodrug Formulations

In cases where compounds are sufficiently basic or acidic to form stable nontoxic acid or base salts, administration of the compound as a pharmaceutically acceptable salt may be appropriate. Examples of pharmaceutically acceptable salts are organic acid addition salts formed with acids, which form a physiological acceptable anion, for example, tosylate, methanesulfonate, acetate, citrate, malonate, tartarate, succinate, benzoate, ascorbate, α-ketoglutarate and α-glycerophosphate. Suitable inorganic salts can also be formed, including but not limited to, sulfate, nitrate, bicarbonate and carbonate salts.

Pharmaceutically acceptable salts can be obtained using standard procedures well known in the art, for example by reacting a sufficiently basic compound such as an amine with a suitable acid, affording a physiologically acceptable anion. Alkali metal (e.g., sodium, potassium or lithium) or alkaline earth metal (e.g., calcium) salts of carboxylic acids can also be made.

The nucleotide prodrugs described herein can be administered to additionally increase the activity, bioavailability, stability or otherwise alter the properties of the nucleotide monophosphate.

A number of nucleotide prodrug ligands are known. In general, alkylation, acylation or other lipophilic modification of the monophosphate or other analog of the nucleoside will increase the stability of the nucleotide.

Examples of substituent groups that can replace one or more hydrogens on the monophosphate moiety are alkyl, aryl, steroids, carbohydrates, including but not limited to sugars, 1,2-diacylglycerol and alcohols. Many are described in R. Jones & N. Bischofberger, Antiviral Research, 1995, 27, 1-17 and S. J. Hecker & M. D. Erion, J. Med. Chem., 2008, 51, 2328-2345. Any of these can be used in combination with the disclosed nucleotides to achieve a desired effect.

The active nucleotide can also be provided as a 5′-phosphoether lipid as disclosed in the following references, which are incorporated by reference: Kucera, L. S., N. Iyer, E. Leake, A. Raben, Modest E. K., D. L. W., and C. Piantadosi, “Novel membrane-interactive ether lipid analogs that inhibit infectious HIV-1 production and induce defective virus formation,” AIDS Res. Hum. Retroviruses, 1990, 6, 491-501; Piantadosi, C., J. Marasco C. J., S. L. Morris-Natschke, K. L. Meyer, F. Gumus, J. R. Surles, K. S. Ishaq, L. S. Kucera, N. Iyer, C. A. Wallen, S. Piantadosi, and E. J. Modest, “Synthesis and evaluation of novel ether lipid nucleoside conjugates for anti-HIV activity,” J. Med. Chem., 1991, 34, 1408-14; Hosteller, K. Y., D. D. Richman, D. A. Carson, L. M. Stuhmiller, G. M. T. van Wijk, and H. van den Bosch, “Greatly enhanced inhibition of human immunodeficiency virus type 1 replication in CEM and HT4-6C cells by 3′-deoxythymidine diphosphate dimyristoylglycerol, a lipid prodrug of 3,-deoxythymidine,” Antimicrob. Agents Chemother., 1992, 36, 2025-29; Hostetler, K. Y., L. M. Stuhmiller, H. B. Lenting, H. van den Bosch, and D. D. Richman, “Synthesis and antiretroviral activity of phospholipid analogs of azidothymidine and other antiviral nucleosides.” J. Biol. Chem., 1990, 265, 61127.

Nonlimiting examples of US patents that disclose suitable lipophilic substituents that can be covalently incorporated into the nucleoside, preferably at R² and/or R³ position of the nucleotides described herein, or lipophilic preparations, include U.S. Pat. No. 5,149,794 (Yatvin et al.); U.S. Pat. No. 5,194,654 (Hostetler et al.), U.S. Pat. No. 5,223,263 (Hostetler et al.); U.S. Pat. No. 5,256,641 (Yatvin et al.); U.S. Pat. No. 5,411,947 (Hostetler et al.); U.S. Pat. No. 5,463,092 (Hostetler et al.); U.S. Pat. No. 5,543,389 (Yatvin et al.); U.S. Pat. No. 5,543,390 (Yatvin et al.); U.S. Pat. No. 5,543,391 (Yatvin et al.); and U.S. Pat. No. 5,554,728 (Basava et al.), all of which are incorporated by reference. Foreign patent applications that disclose lipophilic substituents that can be attached to nucleosides of the present invention, or lipophilic preparations, include WO 89/02733, WO 90/00555, WO 91/16920, WO 91/18914, WO 93/00910, WO 94/26273, WO 96/15132, EP 0 350 287, EP 93917054.4, and WO 91/19721.

### V. Methods of Treatment

Hosts, including but not limited to humans, infected with HCV, Norovirus, Saporovirus, Dengue virus, Chikungunya virus, and/or yellow fever, as well as other viruses in the Caliciviridae or Flaviviridae taxonomic family, or a gene fragment thereof, can be treated by administering to the patient an effective amount of the active compound or a pharmaceutically acceptable prodrug or salt thereof in the presence of a pharmaceutically acceptable carrier or diluent. The active materials can be administered by any appropriate route, for example, orally, parenterally, intravenously, intradermally, subcutaneously, or topically, in liquid or solid form.

In therapeutic use for treating virus infection, the compounds and/or compositions can be administered to patients diagnosed with said virus infection at dosage levels suitable to achieve therapeutic benefit. By “therapeutic benefit,” and grammatical equivalents, is meant the administration of the compound leads to a beneficial effect in the patient over time. For example, therapeutic benefit can be achieved when the virus titer or viral load in a patient is either reduced or stops increasing.

Therapeutic benefit also can be achieved if the administration of a compound slows or halts altogether the onset of adverse symptoms that typically accompany said virus infections, regardless of the virus titer or viral load in the patient. The compounds and/or compositions described herein may also be administered prophylactically in patients who are at risk of developing virus infection, or who have been exposed to virus, to prevent the development of said virus infection. For example, the compounds and/or compositions thereof may be administered to patients likely to have been exposed to said virus.

### VI. Combination or Alternation Therapy

In one embodiment, the compounds of the invention can be employed together with at least one other antiviral agent, chosen from entry inhibitors, reverse transcriptase inhibitors, protease inhibitors, and immune-based therapeutic agents.

For example, when used to treat or prevent HCV infection, the active compound or its prodrug or pharmaceutically acceptable salt can be administered in combination or alternation with another anti-HCV agent, including, but not limited to, those of the formulae above. In general, in combination therapy, effective dosages of two or more agents are administered together, whereas during alternation therapy, an effective dosage of each agent is administered serially. The dosage will depend on absorption, inactivation and excretion rates of the drug, as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions.

Nonlimiting examples of antiviral agents that can be used in combination with the compounds disclosed herein include those in Table 1 below.

TABLE 1
Anti-Hepatitis C Compounds in Current Clinical Development

| | | Pharmaceutical |
|---|---|---|
| Drug Name | Drug Category | Company |
| PEGASYS | Long acting interferon | Roche |
| pegylated interferon | | |
| alfa-2a | | |
| INFERGEN | Interferon, Long acting | InterMune |
| interferon alfacon-1 | interferon | |
| OMNIFERON | Interferon, Long acting | Viragen |
| natural interferon | interferon | |
| ALBUFERON | Longer acting interferon | Human Genome |
| | | Sciences |
| REBIF | Interferon | Ares-Serono |
| interferon beta-1a | | |
| Omega Interferon | Interferon | BioMedicine |
| Oral Interferon alpha | Oral Interferon | Amarillo Biosciences |
| Interferon gamma-1b | Anti-fibrotic | InterMune |
| IP-501 | Anti-fibrotic | Interneuron |
| Merimebodib VX-497 | IMPDH inhibitor | Vertex |
| | (inosine monophosphate | |
| | dehydrogenase) | |
| AMANTADINE | Broad Antiviral Agent | Edo Labs |
| (Symmetrel) | | Solvay |
| IDN-6556 | Apotosis regulation | Idun Pharma. |
| XTL-002 | Monclonal Antibody | XTL |
| HCV/MF59 | Vaccine | Chiron |
| CIVACIR | Polyclonal Antibody | NABI |
| | Therapeutic vaccine | Innogenetics |
| VIRAMIDINE | Nucleoside Analogue | ICN |
| ZADAXIN | Immunomodulator | Sci Clone |
| (thymosin alfa-1) | | |
| CEPLENE | Immunomodulator | Maxim |
| histamine | | |
| dihydrochloride | | |
| VX 950/ | Protease Inhibitor | Vertex/ Eli Lilly |
| LY 570310 | | |
| ISIS 14803 | Antisense | Isis Pharmaceutical/ |
| | | Elan |
| IDN-6556 | Caspase inhibitor | Idun Pharmaceuticals, |
| | | Inc. |
| | | http://www.idun.com |
| JTK 003 | Polymerase Inhibitor | AKROS Pharma |
| Tarvacin | Anti-Phospholipid | Peregrine |
| | Therapy | |
| HCV-796 | Polymerase Inhibitor | ViroPharma/Wye |
| CH-6 | Serine Protease | Schering |
| ANA971 | Isatoribine | ANADYS |
| ANA245 | Isatoribine | ANADYS |
| CPG 10101 (Actilon) | Immunomodulator | Coley |
| Rituximab (Rituxam) | Anti-CD20 Monoclonal | Genetech/IDEC |
| | Antibody | |
| NM283 | Polymerase Inhibitor | Idenix Pharmaceuticals |
| (Valopicitabine) | | |
| HepX ™-C | Monclonal Antibody | XTL |
| IC41 | Therapeutic Vaccine | Intercell |
| Medusa Interferon | Longer acting interferon | Flamel Technologies |
| E-1 | Therapeutic Vaccine | Innogenetics |
| Multiferon | Long Acting Interferon | Viragen |
| BILN 2061 | Serine Protease | Boehringer-Ingelheim |
| Interferon beta-1a | Interferon | Ares-Serono |
| (REBIF) | | |

### VIII. Combination Therapy for Treating Noroviral Infections

In addition to the antiviral compounds described herein, other compounds can also be present. For example, type I interferon (IFN) is known to inhibit Norovirus replication. Certain vitamins, particularly vitamin C, are believed to be effective at treating certain viral infections. One study has shown that Vitamin A supplementation reduced the prevalence of Norovirus GII infections, increased the length of both Norovirus GI and GII shedding, and decreased the prevalence of NoV-associated diarrhea (1: J Infect Dis. 2007 Oct. 1; 196(7):978-85. Epub 2007 Aug. 22). Lysine is known as an antiviral agent. It is also known that virus-like particles (VLPs) derived from genogroup II (GII) Norovirus were bound to cell surface heparan sulfate proteoglycan and other negatively charged glycosaminoglycans. To treat the symptoms of infection, one can also administer an anti-emetic, an anti-diarrheal agent, and/or an analgesic.

### VIII. Pharmaceutical Compositions

Hosts, including but not limited to humans, infected with a Flaviviridae family of viruses or Caliciviridae virus or a gene fragment thereof, can be treated by administering to the patient an effective amount of the active compound or a pharmaceutically acceptable prodrug or salt thereof in the presence of a pharmaceutically acceptable carrier or diluent. The active materials can be administered by any appropriate route, for example, orally, parenterally, intravenously, intradermally, subcutaneously, or topically, in liquid or solid form.

A preferred dose of the compound for will be in the range of between about 0.1 and about 100 mg/kg, more generally, between about 1 and 50 mg/kg, and, preferably, between about 1 and about 20 mg/kg, of body weight of the recipient per day. The effective dosage range of the pharmaceutically acceptable salts and prodrugs can be calculated based on the weight of the parent nucleoside to be delivered. If the salt or prodrug exhibits activity in itself, the effective dosage can be estimated as above using the weight of the salt or prodrug, or by other means known to those skilled in the art.

The compound is conveniently administered in unit any suitable dosage form, including but not limited to but not limited to one containing 7 to 3000 mg, preferably 70 to 1400 mg of active ingredient per unit dosage form. An oral dosage of 50-1000 mg is usually convenient.

Ideally the active ingredient should be administered to achieve peak plasma concentrations of the active compound from about 0.2 to 70 μM, preferably about 1.0 to 15 μM. This can be achieved, for example, by the intravenous injection of a 0.1 to 5% solution of the active ingredient, optionally in saline, or administered as a bolus of the active ingredient.

The concentration of active compound in the drug composition will depend on absorption, inactivation and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient can be administered at once, or can be divided into a number of smaller doses to be administered at varying intervals of time.

A preferred mode of administration of the active compound is oral. Oral compositions will generally include an inert diluent or an edible carrier. They can be enclosed in gelatin capsules or compressed into tablets. For the purpose of oral therapeutic administration, the active compound can be incorporated with excipients and used in the form of tablets, troches or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, unit dosage forms can contain various other materials that modify the physical form of the dosage unit, for example, coatings of sugar, shellac, or other enteric agents.

The compound can be administered as a component of an elixir, suspension, syrup, wafer, chewing gum or the like. A syrup can contain, in addition to the active compound(s), sucrose as a sweetening agent and certain preservatives, dyes and colorings and flavors.

The compound or a pharmaceutically acceptable prodrug or salts thereof can also be mixed with other active materials that do not impair the desired action, or with materials that supplement the desired action, such as antibiotics, antifungals, anti-inflammatories or other antivirals, including but not limited to other nucleoside compounds. Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents, such as ethylenediaminetetraacetic acid; buffers, such as acetates, citrates or phosphates, and agents for the adjustment of tonicity, such as sodium chloride or dextrose. The parental preparation can be enclosed in ampoules, disposable syringes or multiple dose vials made of glass or plastic.

If administered intravenously, preferred carriers are physiological saline or phosphate buffered saline (PBS).

In a preferred embodiment, the active compounds are prepared with carriers that will protect the compound against rapid elimination from the body, such as a controlled release formulation, including but not limited to implants and microencapsulated delivery systems. Biodegradable, biocompatible polymers can be used, such as ethylene vinyl acetate, polyanhydrides, polyglycolic acid, collagen, polyorthoesters and polylactic acid. For example, enterically coated compounds can be used to protect cleavage by stomach acid. Methods for preparation of such formulations will be apparent to those skilled in the art. Suitable materials can also be obtained commercially.

Liposomal suspensions (including but not limited to liposomes targeted to infected cells with monoclonal antibodies to viral antigens) are also preferred as pharmaceutically acceptable carriers. These can be prepared according to methods known to those skilled in the art, for example, as described in U.S. Pat. No. 4,522,811 (incorporated by reference). For example, liposome formulations can be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound or its monophosphate, diphosphate, and/or triphosphate derivatives is then introduced into the container. The container is then swirled by hand to free lipid material from the sides of the container and to disperse lipid aggregates, thereby forming the liposomal suspension.

The terms used in describing the invention are commonly used and known to those skilled in the art. As used herein, the following abbreviations have the indicated meanings:

aq aqueous
CDI carbonyldiimidazole

### DMF N,N-dimethylformamide

DMSO dimethylsulfoxide
EDC 1-ethyl-3-(3-dimethyllaminopropyl)carbodiimide hydrochloride
EtOAc ethyl acetate
h hour/hours

### HOBt N-hydroxybenzotriazole

M molar
min minute
rt or RT room temperature
TBAT tetrabutylammonium triphenyldifluorosilicate
TBTU O-(Benzotriazol-1-yl)-N,N,N′,N′-tetramethyluronium tetrafluoroborate
THF tetrahydrofuran

### IX. General Schemes for Preparing Active Compounds

Methods for the facile preparation of 2,6-diamino 2′-C-Me purine nucleoside monophosphate prodrugs are also provided. The 2,6-diamino 2′-C-Me purine nucleotide monophosphates prodrugs disclosed herein can be prepared as described in detail below, or by other methods known to those skilled in the art. It will be understood by one of ordinary skill in the art that these schemes are in no way limiting and that variations of detail can be made without departing from the spirit and scope of the present invention.

Generally, the nucleoside monophosphate prodrugs of formulas A and B are prepared by first preparing the corresponding nucleoside, then capping the 5′-hydroxy group (and 3′-hydroxy group) as a monophosphate prodrug as described herein that can be readily converted in vivo to the nucleoside monophosphate and ultimately to an active triphosphate form.

The various reaction schemes are summarized below.

Scheme 1 is a non-limiting example of the synthesis of active compounds of the present invention, and in particular, a synthetic approach to nucleosides 1.

Scheme 2 is a non-limiting example of the synthesis of active compounds of the present invention, and in particular, an alternate synthetic approach to nucleosides 1.

Scheme 3 is a non-limiting example of the synthesis of active compounds of the present invention, and in particular, a synthetic approach to monophosphate prodrugs I.

Scheme 4 is a non-limiting example of the synthesis of active compounds of the present invention, and in particular, a synthetic approach to monophosphate prodrugs II.

Scheme 5 is a non-limiting example of the synthesis of active compounds of the present invention, and in particular, a synthetic approach to monophosphate prodrugs III.

Scheme 6 is a non-limiting example of the synthesis of active compounds of the present invention, and in particular, a synthetic approach to monophosphate prodrugs IV-VI.

Scheme 7 is a non-limiting example of the synthesis of active compounds of the present invention, and in particular, a synthetic approach to monophosphate prodrugs VII.

Scheme 8 is a non-limiting example of the synthesis of active compounds of the present invention, and in particular, a synthetic approach to monophosphate prodrugs VIII-IX.

Scheme 9 is a non-limiting example of a pathway to 1′-α-mesylate, 16.

Scheme 10 is a non-limiting example of an alternate pathway to 1′-α-mesylate, 16.

Preparation of compounds of formula A and B is accomplished by first preparing nucleosides 1 which in turn can be accomplished by one of ordinary skill in the art, by methods outlined in: (a) Rajagopalan, P.; Boudinot, F. D; Chu, C. K.; Tennant, B. C.; Baldwin, B. H.; Antiviral Nucleosides: Chiral Synthesis and Chemotheraphy: Chu, C. K.; Eds. Elsevier: 2003. b) Recent Advances in Nucleosides: Chemistry and Chemotherapy: Chu, C. K.; Eds. Elsevier: 2002. c) Frontiers in Nucleosides & Nucleic Acids, 2004, Eds. R. F. Schinazi & D. C. Liotta, IHL Press, Tucker, Ga., USA, pp: 319-37 d) Handbook of Nucleoside Synthesis: Vorbruggen H. & Ruh-Pohlenz C. John Wiley & sons 2001), and by general Schemes 1-2. Specifically, nucleosides 1 can be prepared by coupling sugar 2 with a protected, silylated or free purine base in the presence of Lewis acid such as TMSOTf. Deprotection of the 3′- and 5′-hydroxyls gives nucleoside 1.

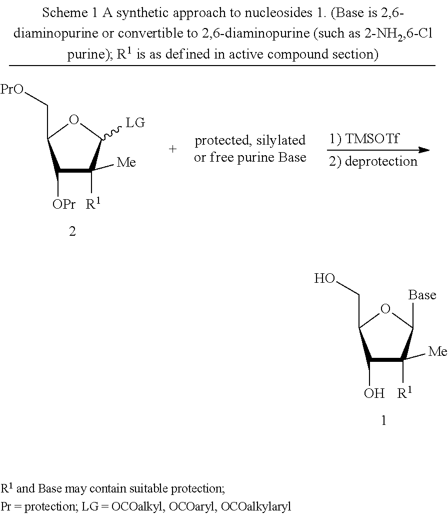

Alternatively, nucleosides 1 could be prepared from 1′-halo, 1′-sulfonate or 1′-hydroxy compounds 3. For the case of 1′-halo or 1′-sulfonate a protected or free purine base in the presence of a base such as triethyl amine or sodium hydride followed by deprotection would give nucleosides 1. For the case of 1′-hydroxy a protected or free purine base in the presence of a Mitsunobu coupling agent such as diisopropyl azodicarboxylate followed by deprotection would give nucleosides 1.

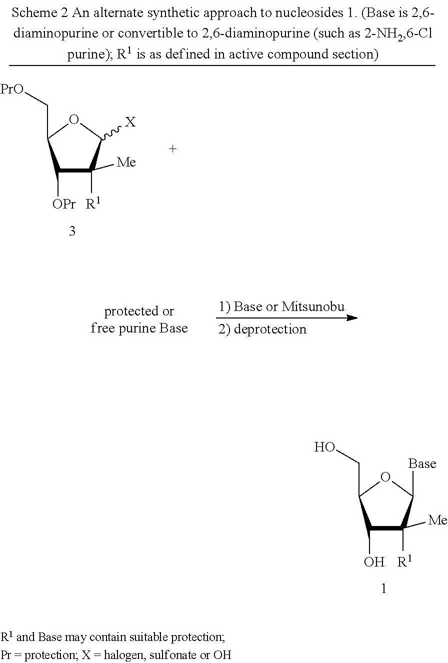

Monophosphate prodrugs I can be prepared as outlined in Scheme 3 starting from phenol 4. Exposure of 4 to phosphorous oxychloride or phosphorothioyl trichloride provides 5, which is subsequently allowed to react with an amino ester 6 to give phosphoramidate 7. Nucleoside 1 can next be converted to monophosphate analog 8 by reaction of the 5′-hydroxyl group with the chlorophosphorylamino propanoate, 7. Removal of protecting groups from the base and/or sugar of 8, if present, provides monophosphate prodrugs I.

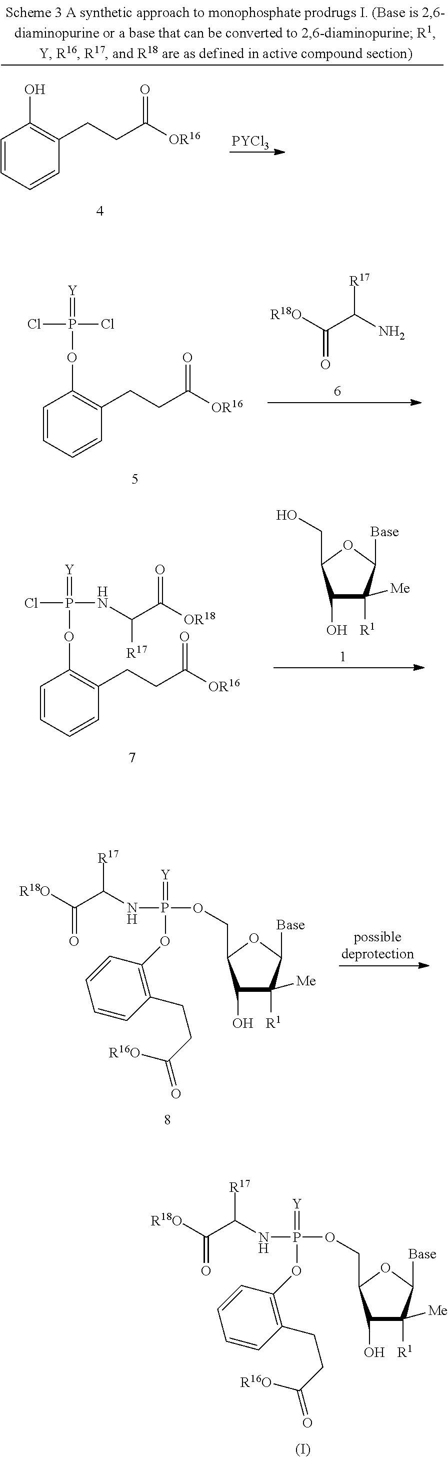

Monophosphate prodrugs II can be prepared by reaction of phenol 4 with phosphorous oxychloride or phosphorothioyl trichloride to provide diphenyl phosphorochloridate, 9 (Scheme 4). Nucleoside 1 can next be converted to an intermediate monophosphate analog by reaction of the 5′-hydroxyl group with the diphenyl phosphorochloridate, 9. Removal of protecting groups, if necessary, provides monophosphate prodrugs II.

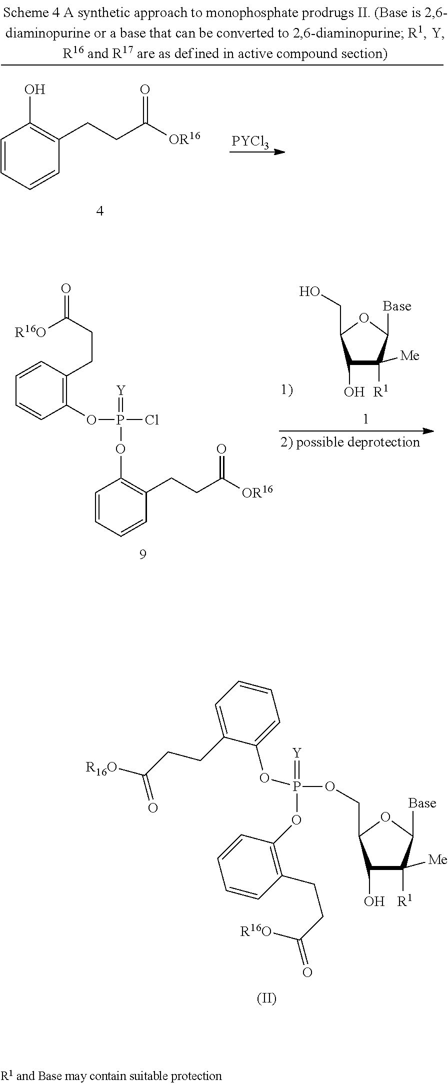

Monophosphate prodrugs III can be prepared by reaction of nucleoside 1 with phosphorous oxychloride or phosphorothioyl trichloride. The resulting intermediate can next be reacted with an L-amino ester followed by water (Scheme 5). Removal of protecting groups, if necessary, provides monophosphate prodrugs III.

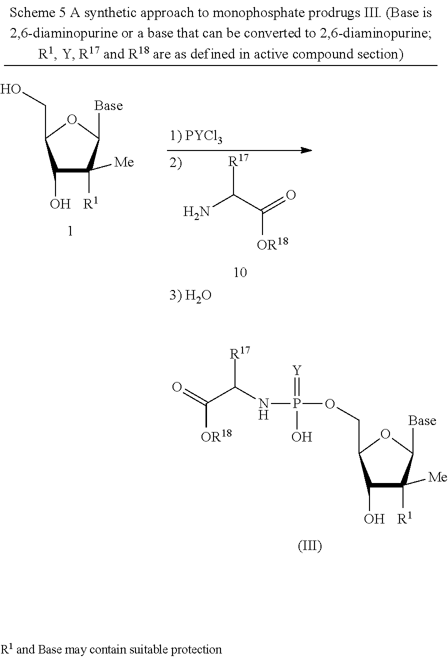

Monophosphate prodrugs IV can be prepared by reaction of nucleoside 1 with phosphorous oxychloride or phosphorothioyl trichloride. The resulting intermediate can next be reacted with an ester of an L-amino acid followed by 11 (Scheme 6). Removal of protecting groups, if necessary, provides monophosphate prodrugs IV. Utilizing a similar protocol with substitution of 10 by R¹⁵OH or 11, monophosphate prodrugs V and VI could also be prepared.

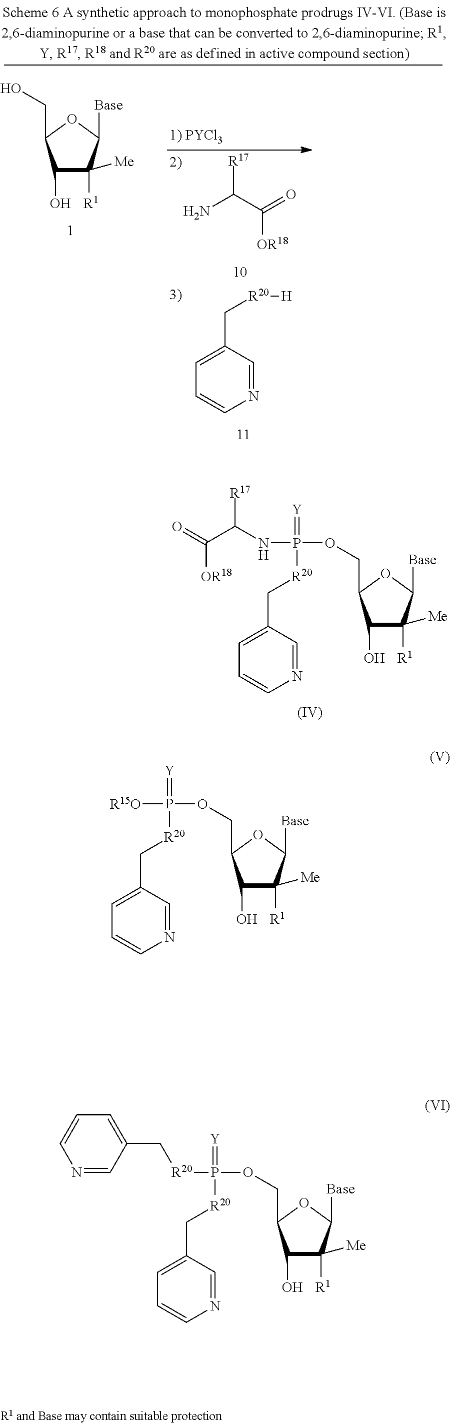

Cyclic phosphate, phosphoramidate, or phosphorodiamidate prodrugs IV can be prepared by reaction of nucleoside 1 with phosphorous oxychloride or phosphorothioyl trichloride. The resulting intermediate can next be reacted with dinucleophile 12 (Scheme 7). Removal of protecting groups, if necessary, provides monophosphate prodrugs VII.

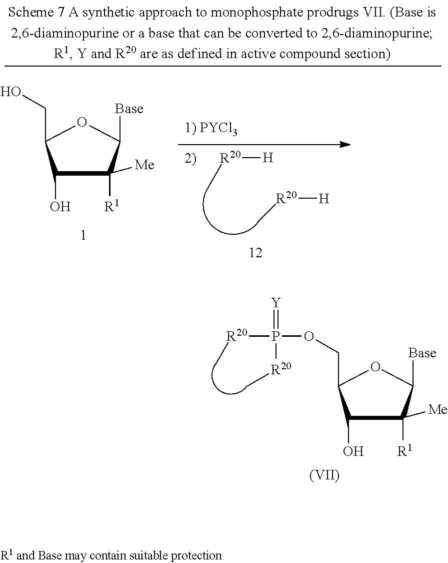

3′,5′-Cyclic phosphate prodrugs VIII can be prepared by reaction of phosphorous oxychloride or phosphorothioyl trichloride with an OH or NH containing reagent such as phenol 4. The resulting intermediate 15 can be purified or used directly with nucleoside 1 (Scheme 8). Removal of protecting groups, if necessary, provides monophosphate prodrugs VIII. Related 3′,5′-cyclic phosphate prodrugs IX maybe prepared in a similar manner from 10, 11, 13 or 14. 3′,5′-Cyclic phosphate prodrugs VIII-IX may also be prepared via known methods involving phosphorous (III) intermediates reacting with 1 followed by oxidation to phosphorous (V) (Scheme 8).

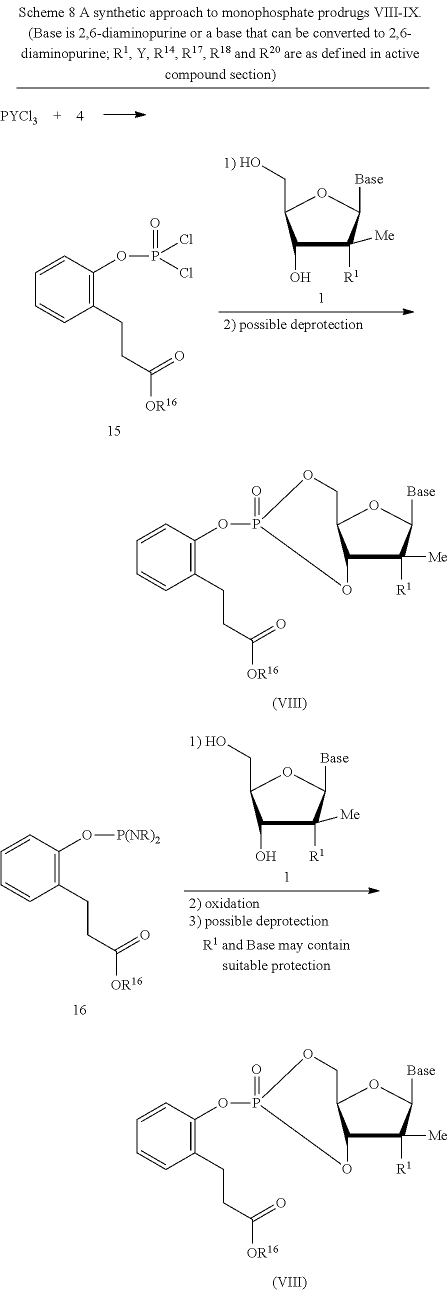

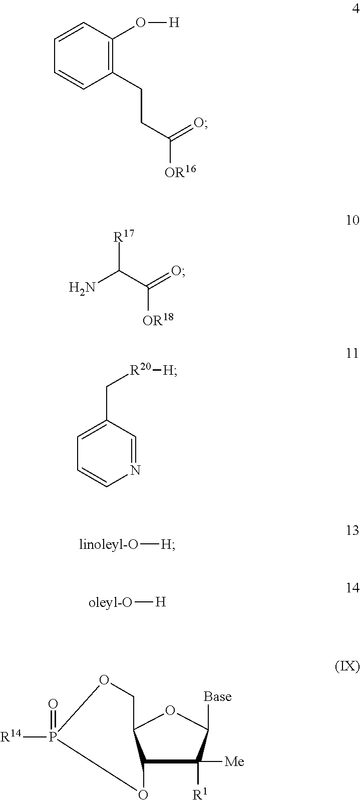

For the case of compound 3 when X=sulfonate (Scheme 2) such as 16 (Scheme 9) which could be prepared from 15 under coupling conditions with a sulfonic acid. For example, coupling conditions such as Mitsunobu coupling with azo carboxylates and phosphorous (III) reagents could provide 16. Compound 15 in the presence of a sulfonic acid or sulfonate salt could be coupled to 15 with diisopropyl azodicarboxylate and triphenylphosphine in a solvent such as dioxane or toluene.

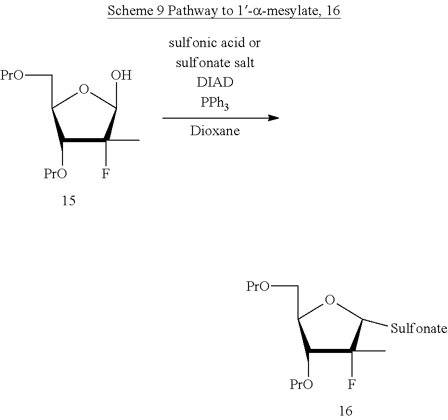

Additionally, sulfonate 16 can be prepared from 15 by first inverting the hydroxy group of 15 by (Scheme 9) coupling conditions such as Mitsunobu coupling with a carboxylic acid or carboxylate salt, an azo carboxylate and a phosphorous (III) reagent could provide 17. Compound 17 in the presence of acetic acid or acetate salt could be coupled to 15 with diisopropyl azodicarboxylate and triphenylphosphine in a solvent such as dioxane or toluene. Selective removal of the acetate of 17 could be preformed with a base such as potassium carbonate in an alcoholic solvent such as methanol to would provide 1′-inverted alcohol 18. Conversion of 16 to 18 could be preformed with a sulfonyl chloride or anhydride in the presence of a base such as triethyl amine or diisopropyl ethyl amine in a solvent such as dichloromethane or dichloroethane.

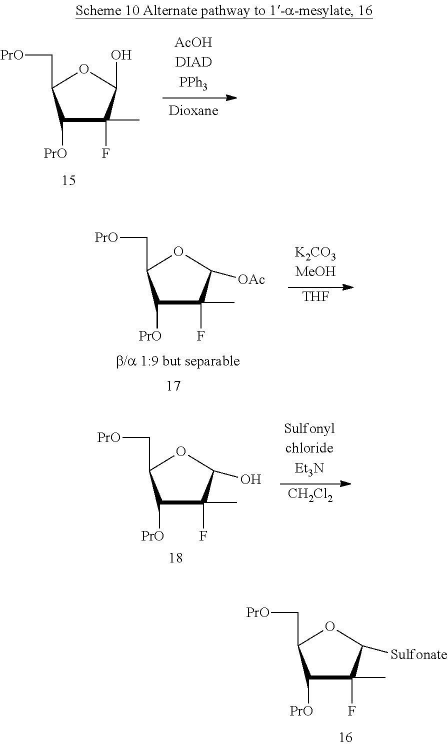

In some cases the phosphorus atom may be chiral herein termed “P*” or “P” which means that and that it has a designation of “R” or “S” corresponding to the accepted meanings of Cahn-Ingold-Prelog rules for such assignment. Prodrugs of Formulas A and B may exist as a mixture of diastereomers due to the chirality at the phosphorus center. When chirality exists at the phosphorous center it may be wholly or partially Rₚ or Sₚ or any mixture thereof.

In another embodiment, the invention relates to a process for preparing a phosphorous analog of an alcohol wherein the phosphorous-oxygen bond is formed by reaction with a reagent of general formulas G or H with a 1°, 2°, or 3° alcohol or 1°, 2°, or 3° alkoxide.

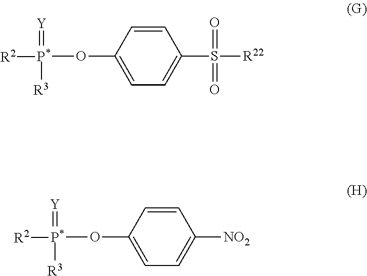

wherein:
the chirality at the phosphorous center of formulas G or H can be wholly or partially Rₚ or Sₚ or any mixture thereof,
Y, R² and R³ are as defined above, and
R²² is, independently, H, C₁₋₂₀ alkyl, CF₃, aryl, such as phenyl, heteroaryl, such as, pyridinyl, substituted aryl, or substituted heteroaryl, or C₁₋₂₀ alkyl substituted with a lower alkyl, alkoxy, di(lower alkyl)-amino, chloro, fluoro, aryl, such as phenyl, heteroaryl, such as, pyridinyl, substituted aryl, or substituted heteroaryl.

In this embodiment, the alcohols are not limited to the purine nucleosides described herein, but can be any alcohols, including, but not limited to, any 5′-OH moiety on a nucleoside with any sugar including such 5′-OH moiety. The compounds formed using this process can be any desired phosphate ester.

In one aspect of this embodiment, where R² and/or R³ of formulas G or H contain a chiral center, the process further involves the step of separating the phosphorous diastereomers by crystallizating the G or H diastereomeric mixture. Where R² and/or R³ of formulas G or H contain a chiral center, the process can further involve the step of separating the phosphorous diastereomers by reacting compounds of formula I with the diastereomeric mixture of formulas G or H,

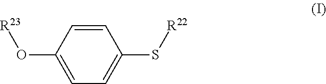

where R²² is as defined above, and

R²³ is selected from H, Li, Na, K, NH₄, and bis salt with Ca or Mg.

Where R² and/or R³ of formulas G or H contain a chiral center, the process can further involve the step of inverting the phosphorous stereocenter by reacting compounds of formula I with a single or enriched diastereomer of formulas G or H.

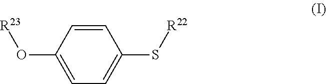

where R²² is as defined above, and

R²³ is selected from H, Li, Na, K, NH₄, and bis salt with Ca or Mg.

The present invention is further illustrated in the following Examples 1-8 which show preparative methods for synthesizing 2,6-diamino 2′-C-Me purine nucleosides and prodrugs, and Examples 9-31 show methods for the biological evaluation of the 2,6-diamino 2′-C-Me purine nucleoside, nucleotide, and nucleotide analogs. It will be understood by one of ordinary skill in the art that these examples are in no way limiting and that variations of detail can be made without departing from the spirit and scope of the present invention.

### SPECIFIC EXAMPLES

Specific compounds which are representative of this invention were prepared as per the following examples and reaction sequences; the examples and the diagrams depicting the reaction sequences are offered by way of illustration, to aid in the understanding of the invention and should not be construed to limit in any way the invention set forth in the claims which follow thereafter. The present compounds can also be used as intermediates in subsequent examples to produce additional compounds of the present invention. No attempt has necessarily been made to optimize the yields obtained in any of the reactions. One skilled in the art would know how to increase such yields through routine variations in reaction times, temperatures, solvents and/or reagents.

Anhydrous solvents were purchased from Aldrich Chemical Company, Inc. (Milwaukee). Reagents were purchased from commercial sources. Unless noted otherwise, the materials used in the examples were obtained from readily available commercial suppliers or synthesized by standard methods known to one skilled in the art of chemical synthesis. Melting points (mp) were determined on an Electrothermal digit melting point apparatus and are uncorrected. ¹H and ¹³C NMR spectra were taken on a Varian Unity Plus 400 spectrometer at room temperature and reported in ppm downfield from internal tetramethylsilane. Deuterium exchange, decoupling experiments or 2D-COSY were performed to confirm proton assignments. Signal multiplicities are represented by s (singlet), d (doublet), dd (doublet of doublets), t (triplet), q (quadruplet), br (broad), bs (broad singlet), m (multiplet). All J-values are in Hz. Mass spectra were determined on a Micromass Platform LC spectrometer using electrospray techniques. Elemental analyses were performed by Atlantic Microlab Inc. (Norcross, Ga.). Analytic TLC was performed on Whatman LK6F silica gel plates, and preparative TLC on Whatman PK5F silica gel plates. Column chromatography was carried out on Silica Gel or via reverse-phase high performance liquid chromatography.

### Example 1

### Synthesis of 2,6-Diamino Purine 2′-C-Me Monophosphate Prodrugs 8a and 8b

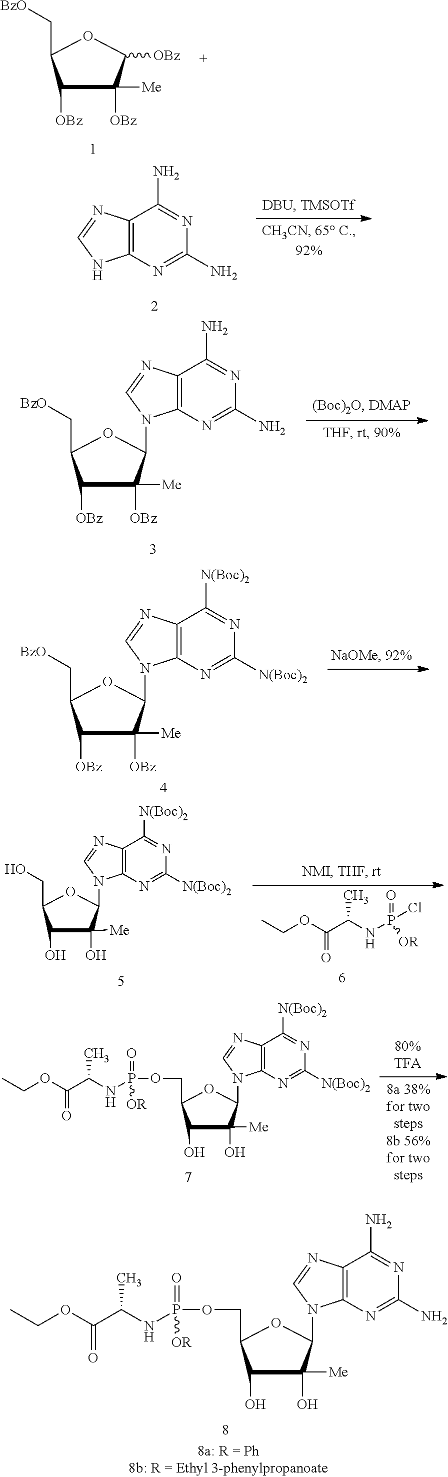

### (2R,3R,4R,5R)-5-((Benzoyloxy)methyl)-2-(2,6-diamino-9H-purin-9-yl)-3-methyltetrahydrofuran-3,4-diyl dibenzoate 3

To a stirred suspension of (3R,4S,5R)-5-((benzoyloxy)methyl)-3-methyltetrahydrofuran-2,3,4-triyl tribenzoate 1 (2.9 g, 5 mmol) and 2,6-diaminopurine 2 (830 mg, 5.5 mmol) in anhydrous acetonitrile at −78° C. was added DBU (2.3 mL, 15.0 mmol), followed by a slow addition of TMSOTf (3.8 mL, 20.0 mmol). The reaction mixture was stirred at −78° C. for 20 min, and then raised to 0° C. After stirred 30 min at 0° C., the reaction mixture was heated gradually to 65° C., and stirred overnight. The reaction mixture was diluted with CH₂Cl₂ (200 mL) and washed with saturated NaHCO₃. The layers were separated and the resulting aqueous layer was extracted with CH₂Cl₂ (2×20 mL). The combined organic layers were dried over Na₂SO₄. After removal the solvent, the residue was purified by silica gel column chromatography (0% to 10% MeOH in EtOAc). 2.8 g of compound 3 was obtained (92% yield). LC/MS calcd. for C₃₂H₂₈N₆O₇ 608.2, observed: 609.2 (M+1).

### (2R,3R,4R,5R)-5-((Benzoyloxy)methyl)-2-(2,6-bis(bis(tert-butoxycarbonyl)amino)-9H-purin-9-yl)-3-methyltetrahydrofuran-3,4-diyl dibenzoate 4

A solution of 3 (1.4 g, 2.3 mmol), Boc anhydride (3.0 g, 13.8 mmol) and DMAP (56 mg, 0.46 mmol) in THF (12 mL) was stirred at rt for 30 h. After the reaction was complete, the solvent was removed under reduced pressure and the residue was purified by flash column chromatography (0% to 40% EtOAc in Hexane). 2.1 g of white solid 4 was obtained (90% yield).

### Di-tert-butyl (9-((2R,3R,4R,5R)-3,4-dihydroxy-5-(hydroxymethyl)-3-methyltetrahydrofuran-2-yl)-9H-purine-2,6-diyl)bis(tert-butoxycarbonylcarbamate) 5

To a solution of 4 (1.7 g, 1.68 mmol) in anhydrous methanol (50 mL) was added a solution of sodium methoxide (4.37 M, 0.3 mL, 1.3 mmol) at rt for 30 min (monitored by TLC and LC-MS). After the reaction was complete, Dowex resin (H⁺ form) was added portion wise to adjust the pH to 7.0. The resin was filtered and washed with methanol, the filtrate was concentrated and the residue was purified by flash column chromatography (0% to 10% MeOH in CH₂Cl₂) to afford 1.08 g white solid 5 (92% yield). ¹H-NMR (CD₃OD): 0.92 (s, 3H, CH₃), 1.40 (s, 18H, 6×CH₃), 1.41 (s, 18H, 6×CH₃), 3.89 (dd, 1H, J=2.8 Hz, J=12.4 Hz), 4.03-4.11 (m, 2H), 4.22 (d, 1H, J=8.8 Hz, H₃′), 6.19 (s, 1H, H₁′), 9.09 (s, 1H, H₈); ¹³C-NMR (CD₃OD): 20.2, 27.9, 28.1, 60.9, 73.1, 80.2, 84.6, 84.9, 85.4, 93.3, 128.8, 147.0, 151.2, 151.9, 152.0, 152.9, 155.0; LC/MS calcd. for C₃₁H₄₈N₆O₁₂ 696.3, observed: 697.4 (M+1).

### (2S)-ethyl 2-((((2S,3R,4R,5R)-5-(2,6-diamino-9H-purin-9-yl)-3,4-dihydroxy-4-methyltetrahydrofuran-2-yl)methoxy)(phenoxy)phosphorylamino)propanoate 8a

To a solution of 5 (780 mg, 1.12 mmol) and N-methylimidazole (0.45 mL, 5.8 mmol) in THF (5 mL) at 0° C. was added dropwise of (2S)-ethyl 2-(chloro(phenoxy)phosphorylamino)propanoate¹ (5.8 mL, 5.8 mmol). The resulting mixture was stirred overnight at rt. After removal of the solvent under reduced pressure, the residue was purified by flash column chromatography (0% to 10% MeOH in CH₂Cl₂) to afford 576 mg of 7a as a white solid (54% yield). A pre-cooled solution (<10° C.) of TFA (80%, 23 mL) was added to a pre-cooled (˜5° C.) 7a (550 mg, 0.58 mmol) in an ice-bath. The solution was stirred from ice-bath temperature to rt, then stirred at rt for 4 h (monitored by TLC and LC/MS). After the reaction was complete, the solvent was removed under reduced pressure and the residue was co-evaporated with methanol (4×15 mL). The residue was dissolved in methanol (20 mL) and neutralized with saturated NaHCO₃. After removal of the solvent, the residue was purified by flash column chromatography (0% to 15% MeOH in CH₂Cl₂) to afford 225 mg white solid 8a (71%) (38.3% yield for two steps). ¹H-NMR (CD₃OD) (1:1 mixture of P diastereomers): 0.94 (s, 3H, CH₃), 0.97 (s, 3H, CH₃), 1.13-1.19 (m, 6H, 2×CH₃), 1.16-1.31 (m, 6H, 2×CH₃), 3.90-4.58 (m, 14H), 5.93 (s, 1H, H₁′), 5.96 (s, 1H, H₁′), 7.14-7.34 (m, 10H, Ar—H), 7.86 (s, 2H, H₈); ³¹PNMR (CD₃OD): 4.77, 4.89; LC/MS calcd. for C₂₂H₃₀N₇O₈P 551.1, observed: 552.3 (M+1).

### Ethyl 3-(2-(((((2S,3R,4R,5R)-5-(2,6-diamino-9H-purin-9-yl)-3,4-dihydroxy-4-methyltetrahydrofuran-2-yl)methoxy)(((S)-1-ethoxy-1-oxopropan-2-yl)amino)phosphoryl)oxy)phenyl)propanoate 8b

A similar procedure was employed for the synthesis of pro-drug 8b. 8b (110 mg) was obtained from 210 mg of 5, 56% for two steps). 8b-up Major (first eluting “up”): Optical rotation [α]²⁴_{D} −7.08 (c 0.24, MeOH); ¹H-NMR (CD₃OD) 0.97 (s, 3H, CH₃), 1.15-1.20 (m, 6H, 2×CH₃), 1.34 (d, 3H, J=7.2 Hz, CH₃), 2.62 (t, 2H, J=8.0 Hz, 2H, CH₂), 2.99 (t, 2H, J=8.0 Hz, 2H, CH₂), 3.95-4.58 (m, 9H), 5.94 (s, 1H, H₁′), 7.07-7.38 (m, 4H, Ar—H), 7.86 (s, 1H, H₈); ³¹PNMR (CD₃OD): 5.03; LC/MS calcd. for C₂₇H₃₈N₇O₁₀P 651.2, observed: 552.2 (M+1). 8b-down Minor (last eluting “down”): Optical rotation [α]²⁴_{D} +12.12 (c 0.13, MeOH); ¹H-NMR (CD₃OD): 0.97 (s, 3H, CH₃), 1.15-1.17 (m, 6H, 2×CH₃), 1.34 (d, 3H, J=7.2 Hz, CH₃), 2.62 (t, 2H, J=8.0 Hz, 2H, CH₂), 2.99 (t, 2H, J=8.0 Hz, 2H, CH₂), 3.96-4.51 (m, 9H), 5.91 (s, 1H, H₁′), 7.10-7.39 (m, 4H, Ar—H), 7.86 (s, 1H, H₈); ³¹PNMR (CD₃OD): 4.98; LC/MS calcd. for C₂₇H₃₈N₇O₁₀P 651.2, observed: 652.3 (M+1).

### REFERENCES

1. (a) Perrone, P.; Daverio, F.; Valente, R.; Rajyaguru, S.; Martin J. A.; Lévêque, V.; Pogam, S. L.; Najera, I.; Klumpp, K.; Smith, D.; B. and McGuigan, C. First Example of Phosphoramidate Approach Applied to a 4′-Substituted Purine Nucleoside (4′-Azidoadenosine): Conversion of an Inactive Nucleoside to a Submicromolar Compound versus Hepatitis C Virus. J. Med. Chem. 2007, 50, 5463-5470. (b) Uchiyama, M.; Aso, Y.; Noyori, R.; Hayakawa, Y. O-Selective phosphorylation of nucleosides without N-protection. J. Org. Chem. 1993, 58, 373-379.

### Example 2

### Synthesis of 2,6-Diamino Purine 2′-C-Me Monophosphate Prodrug 11

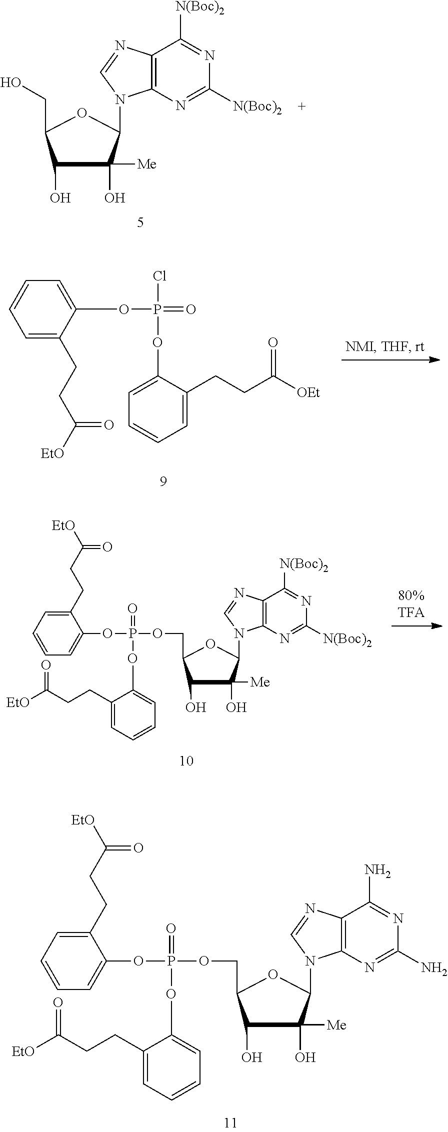

### Ethyl 3-(2-(((((2R,3R,4R,5R)-5-(2,6-diamino-9H-purin-9-yl)-3,4-dihydroxy-4-methyltetrahydrofuran-2-yl)methoxy)(2-(3-ethoxy-3-oxopropyl)phenoxy)phosphoryl)oxy)phenyl)propanoate, 11

To a solution of 5 (630 mg, 0.91 mmol) and N-methylimidazole (0.35 mL, 4.5 mmol) in THF (3 mL) at 0° C. was added dropwise a solution of diethyl 3,3′-(((chlorophosphoryl)bis(oxy))bis(2,1-phenylene))dipropanoate 9 in THF (9 mL, 4.5 mmol). The resulting mixture was stirred overnight at rt. After removed the solvent under reduced pressure, the residue was purified by flash column chromatography in a gradient of MeOH (0% to 10% MeOH in CH₂Cl₂) to afford 540 mg white solid 10 (53% yield). A pre-cold (<10° C.) solution of TFA (80%, 26 mL) was added to a pre-cold (˜5° C.) 10 (540 mg, 0.58 mmol) in an ice-bath. The solution was stirred from 0° C. to rt, then stirred at rt for 4 h (monitored by TLC and LC/MS). After the reaction was complete, the solvent was removed under reduced pressure and the residue was co-evaporated with methanol (4×15 mL). The residue was dissolved in methanol (20 mL) and neutralized by saturated NaHCO₃. After removal of the solvent, the residue was purified by flash column chromatography (0% to 15% MeOH in CH₂Cl₂) to afford 270 mg of 11 as a white solid (77%). LC/MS calcd. for C₂₂H₃₀N₇O₈P 728.2, observed: 729.3 (M+1).

### Example 3

### Alternate Synthesis of 2,6-Diamino Purine 2′-C-Me Monophosphate Prodrug

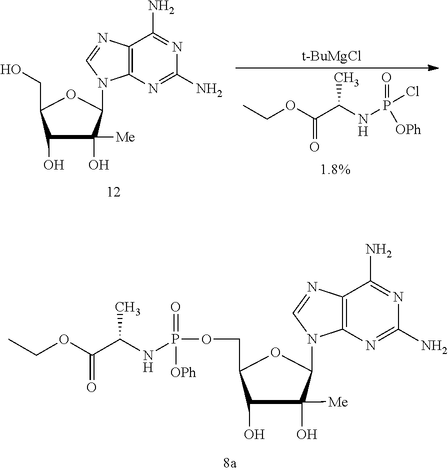

### (2S)-Ethyl 2-((((2R,3R,4R,5R)-5-(2,6-diamino-9H-purin-9-yl)-3,4-dihydroxy-4-methyltetrahydrofuran-2-yl)methoxy)(phenoxy)phosphorylamino)propanoate (8a)

To a solution of 12 (30 mg, 0.1 mmol) in THF (1 mL) and DMF (1 mL) at 0° C. was added (2R)-ethyl 2-(chloro(phenoxy)phosphorylamino)propanoate¹ (0.4 mL, 0.4 mmol), then added t-BuMgCl (0.4 mL, 0.4 mmol) in portions. After stirring for several minutes the reaction was warmed to rt and stirred overnight at rt. The reaction mixture was neutralized with saturated ammonium chloride_{(aq)}, then purified by flash column chromatography (10% to 20% MeOH in CH₂Cl₂) to give 8a (1 mg, 1.8%).

LC/MS calcd. for C₂₂H₃₀N₇O₈P 551.1, observed: 552.1 (M+1).

### REFERENCES

1. (a) Perrone, P.; Daverio, F.; Valente, R.; Rajyaguru, S.; Martin J. A.; Lévêque, V.; Pogam, S. L.; Najera, I.; Klumpp, K.; Smith, D.; B. and McGuigan, C. First Example of Phosphoramidate Approach Applied to a 4′-Substituted Purine Nucleoside (4′-Azidoadenosine): Conversion of an Inactive Nucleoside to a Submicromolar Compound versus Hepatitis C Virus. J. Med. Chem. 2007, 50, 5463-5470. (b) Uchiyama, M.; Aso, Y.; Noyori, R.; Hayakawa, Y. O-Selective phosphorylation of nucleosides without N-protection. J. Org. Chem. 1993, 58, 373-379.

### Example 4

### Synthesis of 17a and 17b; Single Diastereomers for Monophosphate Prodrug Synthesis

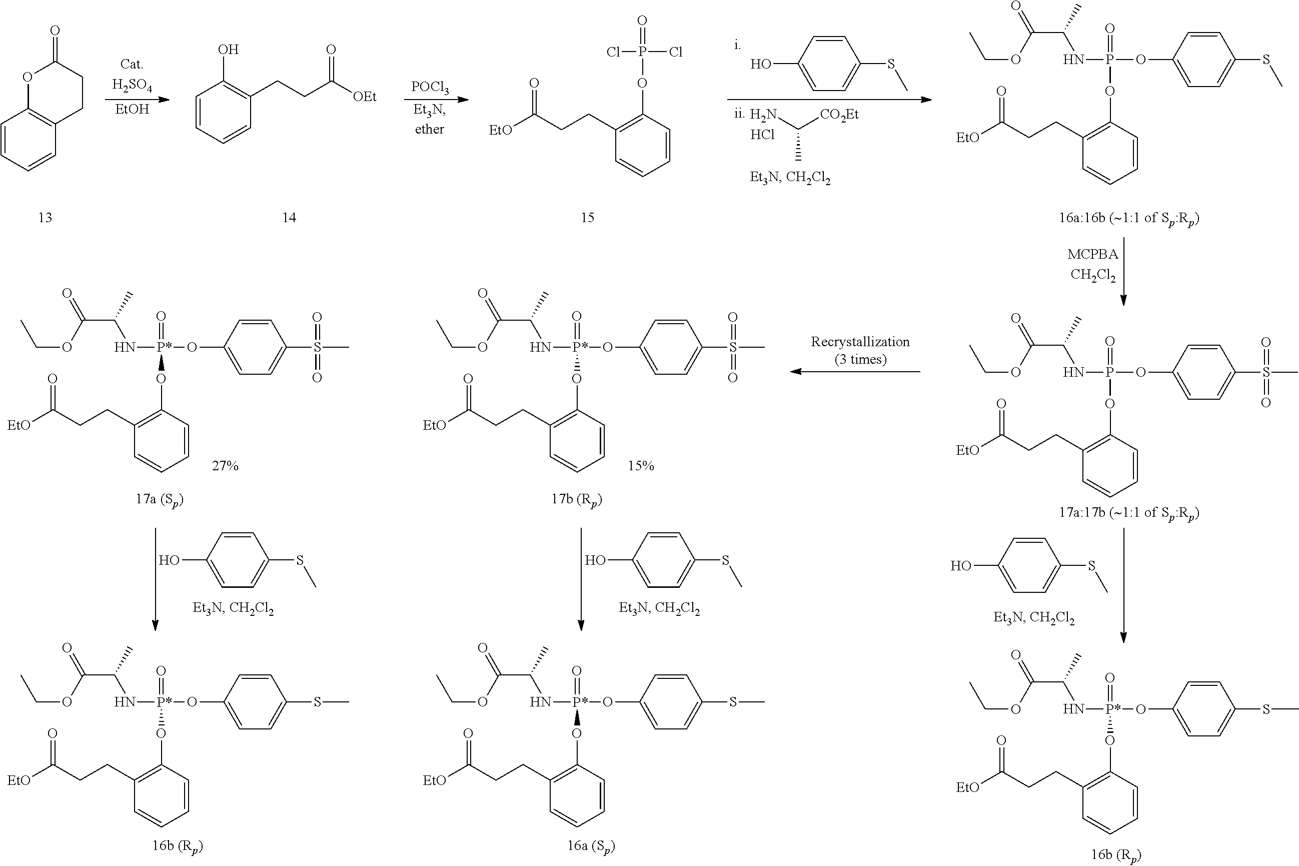

### Ethyl 3-(2-hydroxyphenyl)propanoate, 14

To a solution of dihydrocoumarin 13 (13 g, 87.74 mmol) in 500 mL of anhydrous ethanol was added catalytic conc. H₂SO₄ (0.10 mL) at 0° C. under N₂ atmosphere. The cooling bath was removed and the reaction was stirred for 12 h toward room temperature. The solution was treated with solid NaHCO₃ at 0° C. to pH=6.0-6.5 and the resulting suspension was filtered. The filtrate was concentrated under reduced pressure and purified on a silica gel column to give compound 14 (16.2 g, 83.4 mmol) in 95% yield as yellow oil. ¹H NMR (400 MHz, CDCl₃) δ 7.35 (s, 1H), 7.13-7.07 (m, 2H), 6.89-6.84 (m, 2H), 4.14 (q, J=6.8 Hz, 2H), 2.90 (m, 2H), 2.72 (m, 2H), 1.23 (t, J=6.8 Hz, 3H); ¹³C NMR (100 MHz, CDCl₃) δ 175.89, 154.50, 130.74, 128.15, 127.52, 120.93, 117.33, 61.51, 35.39, 24.84, 14.25; MS-ESI⁺ m/z 195 (M+H⁺)

### Ethyl 3-(2-(((((S)-1-ethoxy-1-oxopropan-2-yl)amino)(4-(methylthio)phenoxy)phosphoryl)oxy)phenyl)propanoate, 16a and 16b: Rₚ and Sₚ mixture (˜1:1)

To a solution of 14 (15.5 g, 79.7 mmol) in 300 mL of anhydrous diethyl ether was added phosphorus oxychloride (12.2 g, 79.7 mmol) and triethylamine (8.5 g, 83.7 mmol) at −78° C. under a N₂ atmosphere. After stirring for 1 h at −78° C. under N₂ atmosphere, the solution was additionally stirred for 12 h toward room temperature then the solid were removed by filtration under a N₂ atmosphere. The filtrate was concentrated under reduced pressure and dried under high vacuum for 6 h at room temperature. To a solution of the resulting sticky oil in 300 mL of anhydrous CH₂Cl₂ was added 4-methylmercaptophenol (11.1 g, 79.0 mmol) and Et₃N (8.0 g, 79.0 mmol) over 20 min at −78° C. under a N₂ atmosphere. Then resulting solution was stirred for 1 h at −78° C. and additionally for 6 h at 0° C. under a N₂ atmosphere. To the solution was added a solution of L-alanine ethyl ester hydrochloride (12.2 g, 79.0 mmol) in 200 mL of anhydrous CH₂Cl₂ and Et₃N (16.2 g, 160 mmol) over 20 min at −78° C. under a N₂ atmosphere. The solution was stirred for 12 h at room temperature and the solids filtered. The filtrate was concentrated under the reduced pressure and purified on a silica gel column (hexane:EtOAc=3:1 to 1:1 v/v) to give compound 16 (33.5 g, 67.7 mmol) in 85% yield in two steps. The ratio of Rₚ and Sₚ mixture was 1:1 by ¹H- and ³¹P-NMR spectra. ¹H NMR (400 MHz, CDCl₃) δ 7.43 (d, J=8.0 Hz, 1H), 7.22-7.15 (m, 6H), 7.15-7.07 (m, 1H), 4.17-3.90 (m, 6H), 2.93 (q, J=8.4 Hz, 2H), 2.58 (m, 2H), 2.45 (s, 3H), 1.39 (t, J=6.4 Hz, 3H), 1.27-1.21 (m, 6H); ³¹P (162 MHz, CDCl₃) δ −2.28, −2.29; MS-ESI⁺ m/z 496 (M+H⁺)

### Ethyl 3-(2-(((((S)-1-ethoxy-1-oxopropan-2-yl)amino)(4-(methylsulfonyl)phenoxy)phosphoryl)oxy)phenyl)propanoate, 17a, 17b: Rₚ and Sₚ mixture (˜1:1)

To a solution of 16 (11.7 g, 23.6 mmol) in 200 mL of anhydrous CH₂Cl₂ was added 3-chloroperoxybenzoic acid (77% maximum, 12.3 g, 53.2 mmol) at 0° C. under a N₂ atmosphere. After stirring for 12 h at room temperature, the solvent was removed under reduced pressure and the residue was dissolved in 200 mL of ethyl acetate and washed with cold saturated NaHCO₃ solution (50 mL×2), cold water (100 mL), and brine (50 mL). The organic layer was dried over Na₂SO₄, filtered and purified on a silica gel column (hexane:EtOAc=3:1 to 1:2 v/v) to give compound 17 (11.7 g, 22.2 mmol) in 94% yield as a mixture of two diastereomers (Rₚ:Sₚ ˜1:1 by ¹H- and ³¹P-NMR spectra). ¹H NMR (400 MHz, CDCl₃) δ 7.92 (m, 2H), 7.48-7.43 (m, 3H), 7.24-7.18 (m, 2H), 7.14-7.10 (m, 1H), 4.53 (m, 1H), 4.19-4.09 (m, 5H), 3.05 (m, 3H), 2.96-2.91 (m, 2H), 2.61-2.56 (m, 2H), 17a: 1.43 (d, J=6.8 Hz, 1.5H), 17b: 1.40 (d, J=6.8 Hz, 1.5H), 1.26-1.21 (m, 6H); ³¹P (162 MHz, CDCl₃) δ −2.50, −2.55; MS-ESI⁺ m/z 528 (M+H⁺)

### Ethyl 3-(2-(((((S)-1-ethoxy-1-oxopropan-2-yl)amino)(4-(methylthio)phenoxy)phosphoryl)oxy)phenyl)propanoate, 16a and 16b

To a solution of compounds 17a and 17b (0.11 g, 0.21 mmol) in 8.0 mL of anhydrous CH₂Cl₂ was added 4-methylmercaptophenol (0.015 g, 0.11 mmol) and Et₃N (0.01 g, 0.12 mmol) at 0° C. under N₂ atmosphere. After stirring for 48 h at room temperature, the solution was concentrated and purified on silica gel (hexane:EtOAc=3:1 to 1:1 v/v) to give 16a and 16b in 19% yield (0.02 g, 0.04 mmol) as the ratio of 1:2 by ¹H NMR spectrum. ¹H NMR (400 MHz, CDCl₃) δ 7.42 (d, J=8.0 Hz, 1H), 7.23-7.15 (m, 6H), 7.11-7.07 (m, 1H), 4.18-4.09 (m, 5H), 3.91-3.83 (m, 1H), 2.92 (q, J=8.0 Hz, 2H), 2.58-2.53 (m, 2H), 2.46 (s, 3H), 1.40 (t, J=6.8 Hz, 3H), 1.26-1.21 (m, 6H); ³¹P (162 MHz, CDCl₃) δ −2.33

### Purification of Rₚ- or Sₚ-isomer from Rₚ/Sₚ-mixture (1:1) of ethyl 3-(2-(((((S)-1-ethoxy-1-oxopropan-2-yl)amino)(4-(methylsulfonyl)phenoxy)phosphoryl)oxy)phenyl)propanoate, 17a/17b

Recrystallization Method

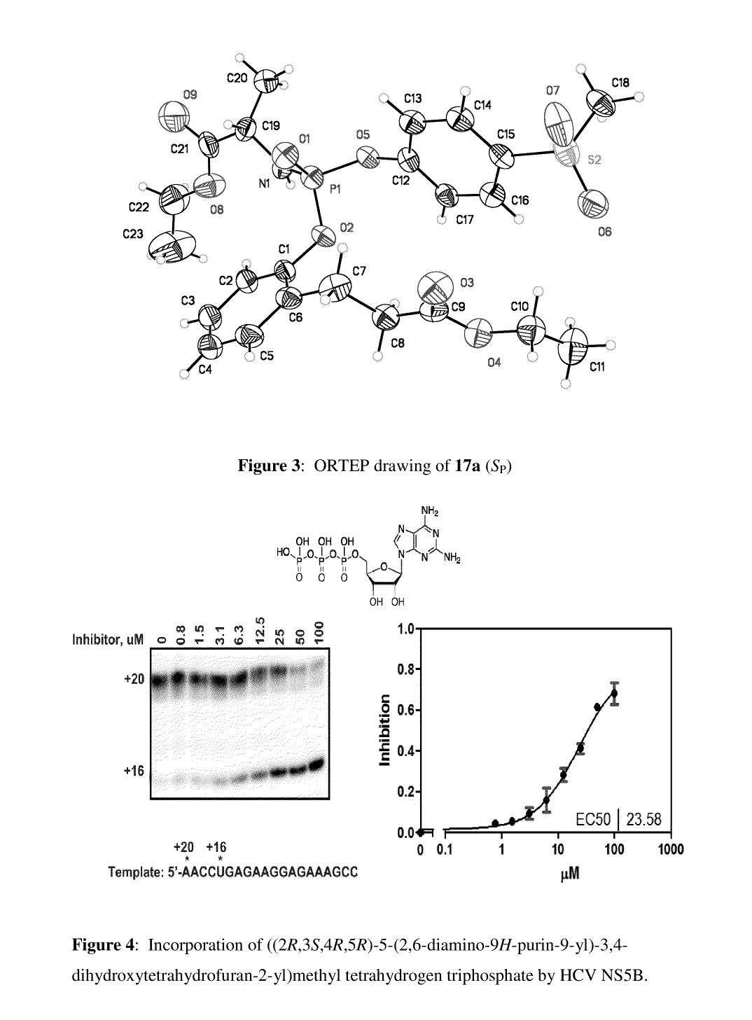

The mixture of two diastereomers 17a and 17b (3.30 g) was dissolved in 50 mL of EtOAc and treated with hexane at room temperature until the solution began to form a white precipitate then keep at 3° C. for 12 h. The white solid was filtered then dried under high vacuum at room temperature for 12 h. The ratio of 17a and 17b in the white solid was 2:1 (2.4 g). The white solid was dissolved in co-solvent (EtOAc:diethyl ether=1:1 v/v, 100 mL) and then stirred for 10 min at room temperature. The solution was treated at room temperature with hexane until a light slurry resulted then stored at 3° C. for 24 h. The white solid was filtered and dried under high vacuum at room temperature for 24 h while the filtrate was used below to obtain 17b. The product 17a (0.90 g, 27%) was obtained in 95% purity based on analysis of the ¹H and ³¹P NMR data. ¹H NMR (400 MHz, CDCl₃) δ 7.92 (d, J=8.8 Hz, 1H), 7.42 (d, J=8.8 Hz, 3H), 7.24-7.19 (m, 2H), 7.14-7.12 (m, 1H), 7.14-7.10 (m, 1H), 4.19-4.11 (m, 5H), 4.02 (m, 1H), 3.05 (s, 3H), 2.94 (m, 2H), 2.58 (dd, J=7.2, 9.6 Hz, 2H), 1.43 (d, J=6.8 Hz, 3H), 1.24 (t, J=6.8 Hz, 6H); ³¹P (162 MHz, CDCl₃) δ −2.68; MS-ESI⁺ m/z 528 (M+H⁺). A single crystal of 17a was obtained by crystallization and an x-ray structure of 17a was obtained unambiguously confirmed the configuration of the phosphorous center as Sₚ (FIG. 3).

The filtrate was concentrated and dried under high vacuum at room temperature for 12 h. The sticky oil was dissolved in 5 mL of CH₂Cl₂ and treated with diisopropyl ether (50 mL) and stirred at room temperature for 10 min. The resulting solution was treated with hexane until a light turbid resulted then stored at 3° C. for 24 h. The white solid was filtered and dried in high vacuum at room temperature for 48 h. The product 17b (0.50 g, 15%) was obtained in 90% purity based on analysis of the ¹H and ³¹P NMR data. ¹H NMR (400 MHz, CDCl₃) δ 7.92 (d, J=8.4 Hz, 2H), 7.45 (d, J=8.4 Hz, 2H), 7.42 (d, J=8.0 Hz, 1H), 7.24-7.19 (m, 2H), 7.14-7.10 (m, 1H), 4.20-4.04 (m, 6H), 3.05 (m, 3H), 2.93 (m, 2H), 2.58 (t, J=7.6 Hz, 2H), 1.40 (d, J=7.2 Hz, 3H), 1.26-1.21 (q, J=7.2 Hz, 6H); ³¹P (162 MHz, CDCl₃) δ −2.60; MS-ESI⁺ m/z 528 (M+H⁺).

### Ethyl 3-(2-(((((S)-1-ethoxy-1-oxopropan-2-yl)amino)(4-(methylthio)phenoxy)phosphoryl)oxy)phenyl)propanoate, 16b

To a solution of compound 17a (0.053 g, 0.10 mmol) in 2.0 mL of anhydrous CH₂Cl₂ was added 4-methylmercaptophenol (0.042 g, 0.30 mmol) and DIEA (0.052 g, 0.04 mmol) at 0° C. under N₂ atmosphere. After stirring for 48 h at room temperature, the solution was concentrated and purified on silica gel (hexane:EtOAc=3:1 to 1:1 v/v) to give 16b (0.047 g, 0.095 mmol) in 95% yield. ¹H NMR (400 MHz, CDCl₃) δ 7.42 (d, J=8.0 Hz, 1H), 7.23-7.15 (m, 6H), 7.11-7.07 (m, 1H), 4.18-4.09 (m, 5H), 3.91-3.83 (m, 1H), 2.92 (q, J=8.0 Hz, 2H), 2.58-2.53 (m, 2H), 2.46 (s, 3H), 1.40 (d, J=6.8 Hz, 3H), 1.26-1.21 (m, 6H); ³¹P (162 MHz, CDCl₃) δ −2.31

### Ethyl 3-(2-(((((S)-1-ethoxy-1-oxopropan-2-yl)amino)(4-(methylthio)phenoxy)phosphoryl)oxy)phenyl)propanoate, 16a

To a solution of compound 17b (0.053 g, 0.10 mmol) in 2.0 mL of anhydrous CH₂Cl₂ was added 4-methylmercaptophenol (0.042 g, 0.30 mmol) and DIEA (0.052 g, 0.04 mmol) at 0° C. under N₂ atmosphere. After stirring for 72 h at room temperature, the solution was concentrated and purified on silica gel (hexane:EtOAc=3:1 to 1:1 v/v) to give 16a (0.045 g, 0.091 mmol) in 91% yield. ¹H NMR (400 MHz, CDCl₃) δ 7.42 (d, J=8.0 Hz, 1H), 7.23-7.15 (m, 6H), 7.11-7.07 (m, 1H), 4.18-4.09 (m, 5H), 3.91-3.83 (m, 1H), 2.92 (q, J=8.0 Hz, 2H), 2.58-2.53 (m, 2H), 2.46 (s, 3H), 1.38 (d, J=7.2 Hz, 3H), 1.26-1.21 (m, 6H); ³¹P (162 MHz, CDCl₃) δ −2.33

### Example 5

### Synthesis of Single Diastereomer 8b-Up from 17a

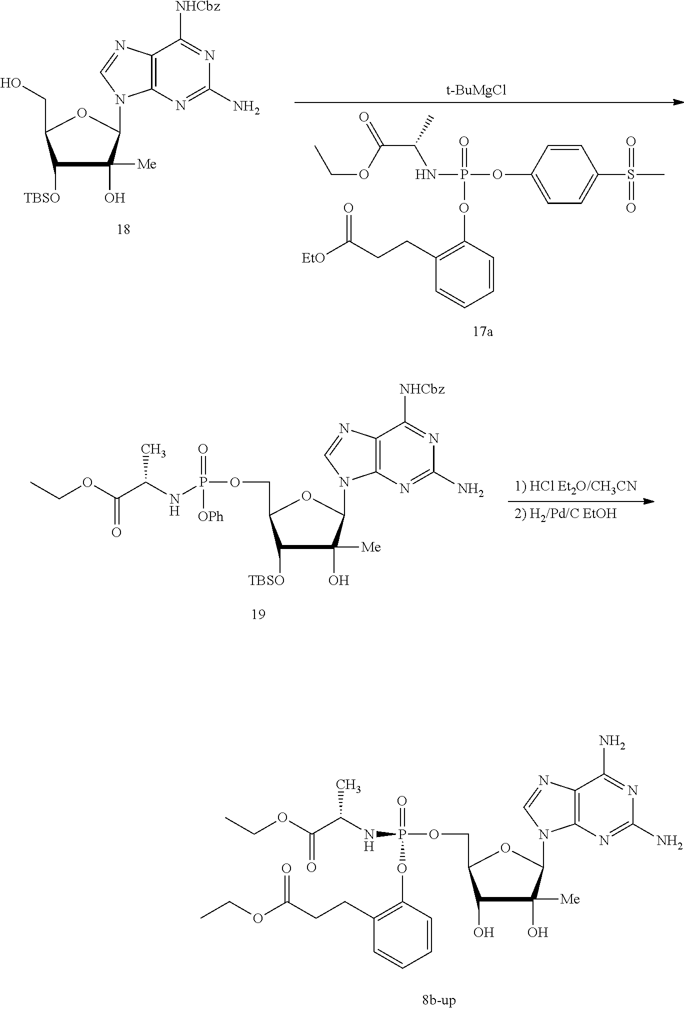

### Ethyl 3-(2-(((((2R,3R,4R,5R)-5-(2-amino-6-(((benzyloxy)carbonyl)amino)-9H-purin-9-yl)-3-((tert-butyldimethylsilyl)oxy)-4-hydroxy-4-methyltetrahydrofuran-2-yl)methoxy)((1-ethoxy-1-oxopropan-2-yl)amino)phosphoryl)oxy)phenyl)propanoate, 19

To a solution of 18 (0.036 g, 0.07 mmol) in 2 mL of anhydrous THF was added t-butylmagnesium chloride (1.0 M in THF, 0.18 mL, 2.5 equiv.) at −78° C. under a N₂ atmosphere. After stirring for 1 h at room temperature, a solution of 17a (0.07 g, 0.14 mmol, 2.0 equiv.) at −78° C. was added to the reaction mixture under a N₂ atmosphere. The reaction mixture was stirred for 48 h at room temperature and treated with saturated NH₄Cl (0.5 mL) at 0° C., and then poured into cold water (10 mL) and extracted with EtOAc (10 mL×3). The collected organic layer was washed brine (10 mL), dried over Na₂SO₄, filtered and purified on a silica gel column (CH₂Cl₂:MeOH=50:1 to 20:1 v/v) to give compound 19 (0.025 g, 0.028 mmol) in 40% yield. ¹H NMR (400 MHz, CDCl₃) δ 8.16 (s, 1H), 7.83 (s, 1H), 7.44-7.33 (m, 6H), 7.21-7.05 (m, 3H), 5.99 (s, 1H), 5.27 (s, 2H), 5.22 (s, 2H), 4.66-4.61 (m, 1H), 4.42 (d, J=8.0 Hz, 1H), 4.39-4.34 (m, 1H), 4.16-3.97 (m, 7H), 3.85 (m, 1H), 3.19 (s, 1H), 3.01 (m, 2H), 2.66 (m, 2H), 1.86 (m, 1H), 1.26 (d, J=6.8 Hz, 3H), 1.22-1.14 (dt, J=14.4, 7.2 Hz, 6H), 0.94 (s, 3H), 0.93 (s, 9H), 0.19 (s, 3H), 0.13 (s, 3H); ³¹P (162 MHz, CDCl₃) δ 3.40; MS-ESI⁺ m/z 900 (M+H⁺)

### Ethyl 3-(2-(((((2R,3R,4R,5R)-5-(2,6-diamino-9H-purin-9-yl)-3,4-dihydroxy-4-methyltetrahydrofuran-2-yl)methoxy)((1-ethoxy-1-oxopropan-2-yl)amino)phosphoryl)oxy)phenyl)propanoate, 8b-Up

To a solution of 19 (0.01 g, 0.011 mmol) in 2.0 mL of anhydrous CH₃CN was added hydrogen chloride (2.0 M in diethyl ether, 1.0 mL) at 0° C. After stirring for 48 h at room temperature, the solvent and hydrogen chloride was removed under reduced pressure. The residue was washed with diethyl ether (5 mL×5) and dried at high vacuum for 12 h at room temperature. The solid was dissolved in 2.0 mL of EtOH and stirred for 30 min at room temperature. To the solution was added Pd/C (5.0 mg, 10% Pd on carbon) and resulting suspension was stirred for 12 h under hydrogen atmosphere (1 atm) at room temperature. The solution was treated with Celite (0.05 g) and filtered. The filtrate was concentrated under reduced pressure and purified on silica gel column (CH₂Cl₂:MeOH=10:1 v/v) to give compound 8b-up (0.007 g, 0.001 mmol) in 91% yield. ¹H NMR (400 MHz, CD₃OD) δ 7.82 (s, 1H), 7.33 (d, J=8.4 Hz, 1H), 7.22 (d, J=7.2 Hz, 1H), 7.13 (td, J=7.6, 2.0 Hz, 1H), 7.06 (t, J=7.6 Hz, 1H), 5.90 (s, 1H), 4.60-4.53 (m, 1H), 4.48-4.20 (m, 1H), 4.10-4.04 (m, 2H), 4.02 (q, J=7.6 Hz, 2H), 3.96-3.86 (m, 1H), 2.96 (t, J=8.0 Hz, 2H), 2.59 (t, J=8.0 Hz, 2H), 1.30 (dd, J=1.2, 7.2 Hz, 3H), 1.16 (t, J=7.8 Hz, 3H), 1.13 (t, J=7.2 Hz, 3H), 0.93 (s, 3H); ³¹P (162 MHz, CD₃OD) δ 5.01; MS-ESI⁺ m/z 652 (M+H⁺).

### Example 6

### Synthesis of Phosphoramidate Prodrugs (Sp)-8b-Down and (Rp)-8b-Up from (Rp)-24 and (Sp)-25 Respectively

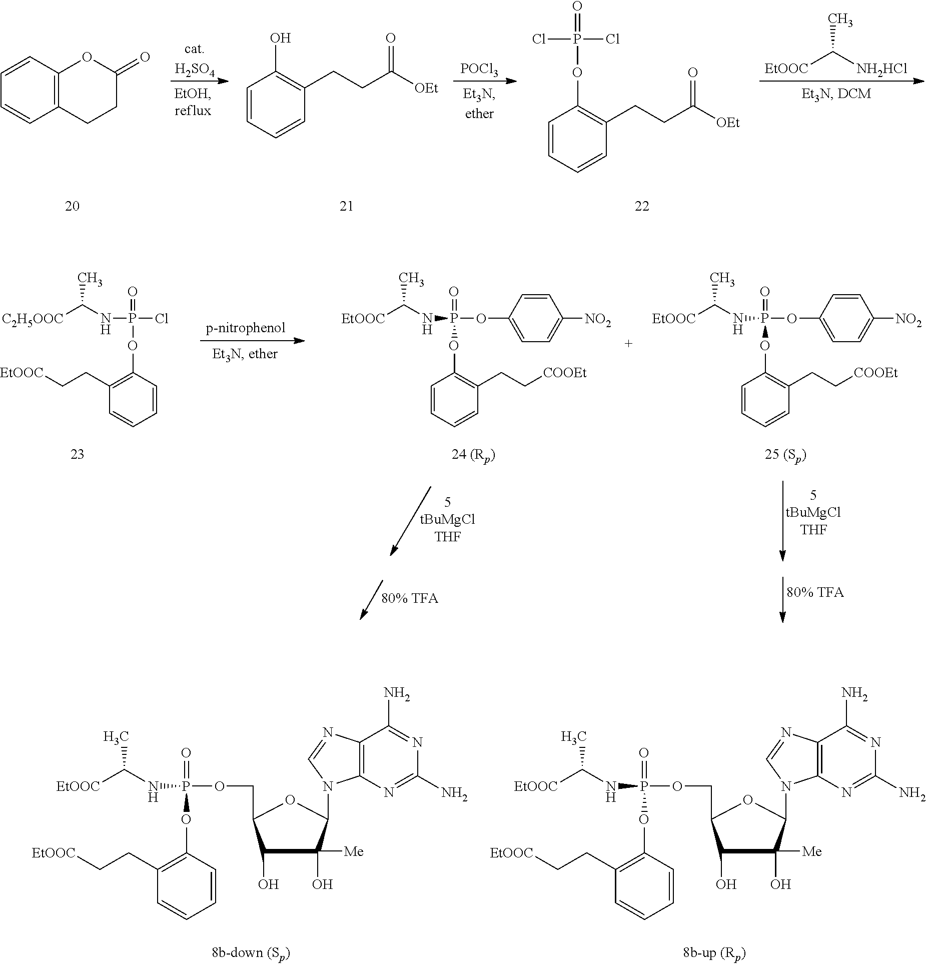

### Ethyl-3-(2-hydroxyphenyl)propionate, 21

Dihydrocoumarin, 20 (10.4 g, 70.0 mmol) was added to 60 mL dry ethanol. H₂SO₄ (0.1 mL) was added and the resulting solution was heated overnight at reflux. The ethanol was removed under reduced pressure, the residue was dissolved in diethyl ether and the organic phase was extracted with sodium bicarbonate solution. The organic phase was dried with sodium sulfate, the solvent was evaporated and the residue was subjected to chromatography on silica gel (MeOH/CH₂Cl₂, MeOH gradient 0 to 10%). The product, 21, was isolated as colorless needles (80% yield). ¹H NMR (400 MHz, CDCl₃) δ 7.40 (s, 1H), 7.05-7.15 (m, 2H), 6.84-6.90 (m, 2H), 4.14 (q, J=6.8 Hz, 2H), 2.90 (m, 2H), 2.72 (m, 2H), 1.23 (t, J=6.8 Hz, 3H); LC-MS, m/z 195 (M+1)⁺.

### Ethyl 3-(2-((chloro(((R)-1-ethoxy-1-oxopropan-2-yl)amino)phosphoryl)oxy)phenyl)propanoate, 23

A solution of 21 (5.0 g, 25.7 mmol) and triethylamine (3.6 mL, 25.7 mmol) in 80 mL of anhydrous diethyl ether was added dropwise to a −78° C. solution of phosphorus oxychloride (2.4 mL, 25.7 mmol) in 70 mL of anhydrous diethyl ether under an Ar atmosphere over 2 h. After stirring for 1 h at −78° C. under Ar atmosphere, the solution was additionally stirred for 15 h toward room temperature then the solids were removed by filtration under a N₂ atmosphere. The solids were washed anhydrous diethyl ether and the combined filtrate was concentrated under reduced pressure then dried under high vacuum overnight at room temperature to provide 22 as a colorless oil that was used without further purification.

To a mixture of 22 and pre-dried L-alanine ethyl ester hydrochloride (3.94 g, 25.7 mmol) in 20 mL of anhydrous CH₂Cl₂ at −78° C. under Ar atmosphere was added a solution of Et₃N (7 mL, 51.4 mmol) in 20 mL of anhydrous CH₂Cl₂ over 2 h. The solution was stirred for 16 h at room temperature and the solids were filtered. The filtrate was concentrated under the reduced pressure and purified on a silica gel column (EtOAc/hexane, EtOAc gradient 0 to 50%, v/v) to give 9.52 g of compound 23 as an almost colorless oil in 75% yield for two steps. Compound 23 could be stored for long periods without noticeable degradation by preparing 1 M solution in THF and storing over 4 Å sieves at −70° C. ¹H NMR (400 MHz, CDCl₃) δ 7.14-7.49 (m, 4H), 4.70-4.80 (m, 1H), 4.09-4.27 (m, 5H), 2.92-3.08 (m, 2H), 2.61-2.65 (m, 2H), 1.50-1.55 (m, 3H), 1.21-1.32 (m, 6H). ³¹P NMR (162 MHz, CDCl₃) δ 8.88, 8.72.

### Ethyl 3-(2-(((R)—(((S)-1-ethoxy-1-oxopropan-2-yl)amino)(4-nitrophenoxy)phosphoryl)oxy)phenyl)-propanoate 24 (Rₚ) and ethyl 3-(2-(((S)—(((S)-1-ethoxy-1-oxopropan-2-yl)amino)(4-nitrophenoxy)-phosphoryl)oxy)phenyl)propanoate 25 (Sₚ)

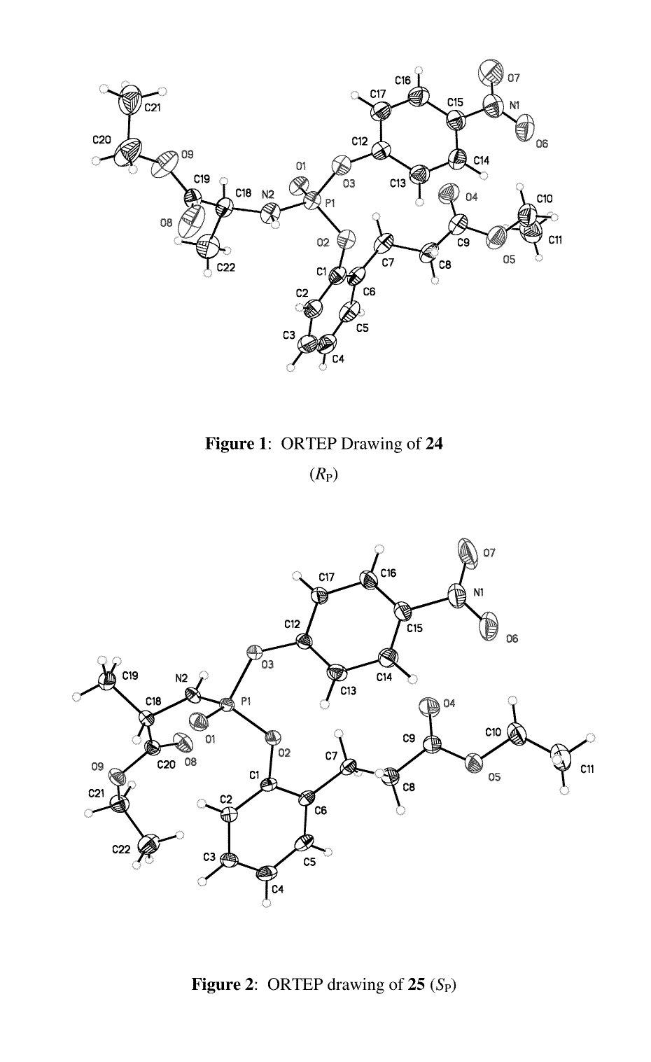

A solution of Et₃N in anhydrous diethyl ether (100 mL) was added dropwise to a solution of 23 (10.0 g, 25.6 mmol) and p-nitro phenol (3.75 g, 27.0 mmol) in diethyl ether (200 mL) at 0° C. over 30 min. The reaction mixture was stirred at 0° C. for 1 h then toward room temperature for 15 h. The solids were filtered and the filtrate was concentrated under the reduced pressure. The residue was purified on a silica gel column (EtOAc/CH₂Cl₂, EtOAc gradient 0 to 10%, v/v) to give 10.8 g of a mixture of 24 and 25 in 85% yield in ˜1:1 ratio. The mixture was recrystallized in 2% CH₃CN in diisopropyl ether with crystalline 24 as seed crystals which were obtained by silica gel column chromatography. Diastereomer 24 was collected by filtration (2.2 g, >20:1 24:25). ¹H NMR (400 MHz, CDCl₃) δ 8.22-8.24 (dd, J=10 Hz, J=2.0 Hz, 2H), 7.11-7.44 (m, 6H), 4.06-4.20 (m, 6H), 2.88-3.00 (m, 2H), 2.54-2.59 (m, 2H), 1.40 (d, J=6.8 Hz, 3H), 1.25 (t, J=7.2 Hz, 3H), 1.23 (t, J=7.2 Hz, 3H). ³¹P NMR (162 MHz, CDCl₃) δ −2.01. LC-MS, m/z 495 (M+1)⁺. A single crystal of 24 was obtained by crystallization in 2% CH₃CN in diisopropyl ether and an x-ray structure of 24 was obtained unambiguously confirmed the configuration of the phosphorous center as Rₚ (FIG. 1).

The filtrate was concentrated under reduced pressure to a residue pressure then dried under high vacuum overnight at room temperature. The residue was dissolved in diisopropyl ether (200 mL) with gentle heating and seed crystals of 25 were added. After setting at room temperature for 3 days 25 (510 mg, ˜20:1 24:25) was collected by filtration. ¹H NMR (400 MHz, CDCl₃) δ 8.22-8.24 (dd, J=10 Hz, 2H), 7.11-7.43 (m, 6H), 4.00-4.18 (m, 6H), 2.93-2.98 (m, 2H), 2.55-2.60 (m, 2H), 1.43 (d, J=7.2 Hz, 3H), 1.24 (t, J=7.2 Hz, 3H), 1.24 (t, J=7.2 Hz, 3H). ³¹P NMR (162 MHz, CDCl₃) δ −2.07. LC-MS, m/z 495 (M+1)⁺. A single crystal of 25 was obtained by crystallization and an x-ray structure of 25 was obtained unambiguously confirmed the configuration of the phosphorous center as Sₚ (FIG. 2).

### Ethyl 3-(2-(((R)-(((2R,3R,4R,5R)-5-(2,6-diamino-9H-purin-9-yl)-3,4-dihydroxy-4-methyltetrahydrofuran-2-yl)methoxy)(((S)-1-ethoxy-1-oxopropan-2-yl)amino)phosphoryl)oxy)phenyl)propanoate, 8b-up (R_{P})

To a solution of 5 (100 mg, 0.14 mmol) in THF (0.5 mL) was added 0.5 mL of t-BuMgCl solution (1M, 0.5 mmol) at −78° C. under Ar atmosphere. The reaction mixture was stirred for 30 min at this temperature and then warm to room temperature. A solution of 13 (210 mg, 0.42 mmol) in 1 mL of anhydrous THF was added. The reaction mixture was stirred at room temperature for 3 days under Ar atmosphere for completion. Solvent was evaporated under reduced pressure, and the residue was added a pre-cooled 80% TFA solution (10 mL) at 0° C. The reaction mixture was additionally stirred for 4 h toward room temperature for completion. After evaporated the solvents under reduced pressure, the residue was added small amount of saturated NaHCO₃ to pH 7.0. The mixture was concentrated under reduced pressure and then purified on silica gel column (MeOH/DCM, MeOH gradient 0 to 10%, v/v) to afford 37.5 mg 8b-up (Rₚ) in 41% in two steps. Optical rotation [α]²⁴_{D} −7.08 (0.24, MeOH); ¹HNMR (400 MHz, CD₃OD) δ 0.97 (s, 3H, CH₃), 1.15-1.20 (m, 6H, 2×CH₃), 1.34 (d, 3H, J=7.2 Hz, CH₃), 2.62 (t, 2H, J=8.0 Hz, 2H, CH₂), 2.99 (t, 2H, J=8.0 Hz, 2H, CH₂), 3.95-4.58 (m, 9H), 5.94 (s, 1H, H₁′), 7.07-7.38 (m, 4H, Ar—H), 7.86 (s, 1H, H₈); ¹³CNMR (100 MHz, CD₃OD) δ 14.5, 14.6, 20.4, 20.6, 26.8, 35.4, 51.7, 61.7, 62.5, 67.0, 74.4, 80.1, 81.9, 92.8, 114.4, 121.0, 126.2, 128.8, 131.8, 133.2, 137.5, 150.6, 152.7, 157.7, 162.0, 174.8, 175.1; ³¹PNMR (162 MHz, CD₃OD): 5.03; LC/MS calcd. for C₂₇H₃₈N₇O₁₀P 651.2, observed: 552.2 (M+1).

### Ethyl 3-(2-(((S)-(((2R,3R,4R,5R)-5-(2,6-diamino-9H-purin-9-yl)-3,4-dihydroxy-4-methyltetrahydrofuran-2-yl)methoxy)(((S)-1-ethoxy-1-oxopropan-2-yl)amino)phosphoryl)oxy)phenyl)propanoate, 8b-down (S_{P})

Similar procedure was employed for the preparation of 8b-down in 39% yield. Optical rotation [α]²⁴_{D} +12.12 (0.13, MeOH); ¹HNMR (400 MHz, CD₃OD) δ 0.97 (s, 3H, CH₃), 1.15-1.17 (m, 6H, 2×CH₃), 1.34 (d, 3H, J=7.2 Hz, CH₃), 2.62 (t, 2H, J=8.0 Hz, 2H, CH₂), 2.99 (t, 2H, J=8.0 Hz, 2H, CH₂), 3.96-4.51 (m, 9H), 5.93 (s, 1H, H₁′), 7.10-7.39 (m, 4H, Ar—H), 7.86 (s, 1H, H₈); ¹³CNMR (100 MHz, CD₃OD) δ 14.5, 14.6, 20.4, 20.8, 26.8, 35.4, 51.6, 61.6, 62.4, 67.7, 74.7, 80.0, 82.1, 93.0, 114.4, 121.1, 126.2, 128.8, 131.7, 133.1, 137.7, 150.5, 152.6, 157.6, 161.9, 174.7, 174.8; ³¹PNMR (162 MHz, CD₃OD): 4.98; LC/MS calcd. for C₂₇H₃₈N₇O₁₀P 651.2, observed: 652.3 (M+1).

### Example 7

### Synthesis of Ethyl Panthenoate Single Diastereomer Prodrug 30

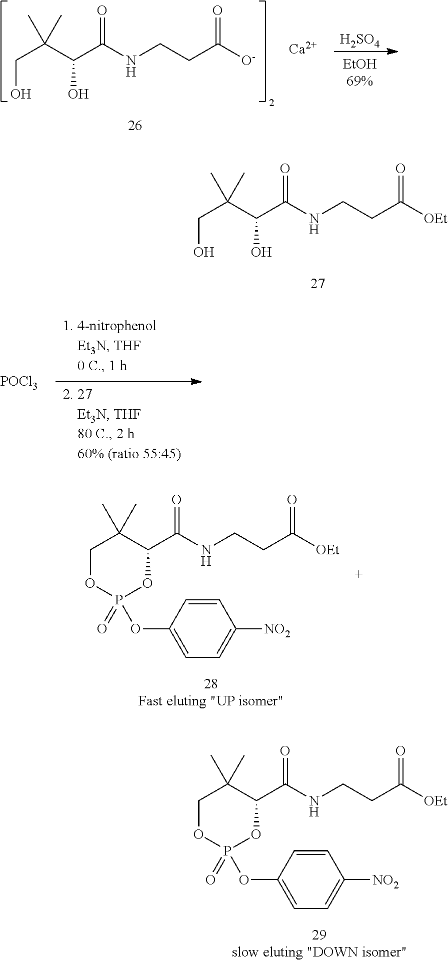

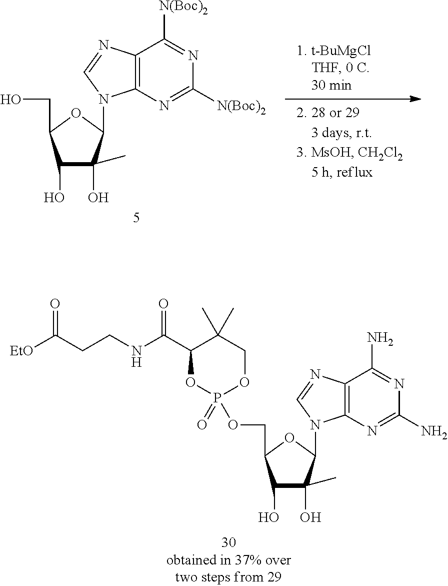

### (R)-Ethyl 3-(2,4-dihydroxy-3,3-dimethylbutanamido)propanoate, 27

To a stirred suspension of panthenoate calcium 26 (10 g, 42 mmol) in ethanol (200 mL) was added a catalytic amount of sulfuric acid and the mixture was heated to reflux overnight. The mixture was filtrated and neutralized by addition of a saturated NaHCO₃ solution (50 mL). Ethanol was removed by evaporation under reduced pressure and aqueous phase was extracted EtOAc (30 mL×5). The combined organic layers were dried over Na₂SO₄, filtrated and evaporated to give 27 (7.1 g, 28.7 mmol) as a slightly yellow oil. ¹H-NMR (400 MHz, CDCl₃) δ ppm 0.87 (s, 3H), 0.95 (s, 3H), 1.24 (t, J=7.1 Hz, 3H), 2.53 (t, J=6.2 Hz, 2H), 3.60-3.43 (m, 4H), 3.91 (s, 1H), 3.98 (s, 1H), 4.12 (q, J=7.1 Hz, 2H), 4.47 (s, 1H), 7.33 (t, J=5.7 Hz, 1H). LC/MS calcd. For C₁₁H₂₂NO₅ 248.1, observed: 248.1 (M+1).

### Ethyl 3-((4R)-5,5-dimethyl-2-(4-nitrophenoxy)-2-oxido-1,3,2-dioxaphosphinane-4-carboxamido)propanoate, 28 and 29

To stirred solution of POCl₃ (1 mmol, 83 μL) in THF (5 mL) at 0° C. was added a solution of 4-nitrophenol (1 mmol, 139 mg) and Et₃N (1 mmol, 139 μL) in THF (1 mL). After stirring 1 h at room temperature, the mixture was added to a solution of B (0.81 mmol, 200 mg) and Et₃N (2 mmol, 83 μL) in THF (10 mL). The resulting mixture was heated at 80° C. for 2 h. The solution was hydrolyzed by a 10% water solution of NaHCO₃ and extracted three times by EtOAc. The combined organic layers were washed with brine and dried over Na₂SO₄. After removal of the solvent, the residue was purified by silica gel column chromatography (50% EtOAc in hexanes for the fast eluting diastereomer, then 65% EtOAc in hexanes for the slow eluting diastereomer) to give the fast eluting diastereomer 28 (0.23 mmol, 100 mg) and the slow eluting diastereomer 29 (0.27 mmol, 115 mg) in a 60% overall yield.

28, fast eluting diastereomer. ¹H-NMR (400 MHz, CD₃OD) δ ppm 1.08 (s, 3H), 1.13 (s, 3H), 1.18 (t, J=7.1 Hz, 3H), 2.52 (t, J=6.7 Hz, 2H), 3.39-3.53 (m, 2H), 4.00-4.10 (m, 3H), 4.42 (d, J=11.4 Hz, 1H), 4.88 (s, 1H), 7.47 (d, J=9.2 Hz, 2H), 8.24-8.28 (m, 2H); ³¹PNMR (CD₃OD): −14.02; LC/MS calcd. for C₁₇H₂₄N₂O₉P 431.1, observed: 431.1 (M+1). Optical rotation [α]²⁴_{D} +51.08 (c 0.184, MeOH)

29, slow eluting diastereomer. ¹H-NMR (400 MHz, CD₃OD) δ ppm 0.89 (s, 3H), 1.18-1.23 (m, 6H), 2.49 (t, J=6.5 Hz, 2H), 3.45 (dt, J=6.7, 2.4 Hz, 2H), 4.10 (q, J=7.1 Hz, 2H), 4.22 (t, J=11.8 Hz, 1H), 4.57 (dd, J=12.7, 11.1 Hz, 1H), 4.73 (d, J=10.3 Hz, 1H), 7.50 (dd, J=9.14, 0.93 Hz, 2H), 8.26-8.32 (m, 2H); ³¹PNMR (CD₃OD): −13.31; LC/MS calcd. for C₁₇H₂₄N₂O₉P 430.1, observed: 431.1 (M+1). Optical rotation [α]²⁴_{D} +46.94 (c 0.196, MeOH)

### Ethyl 3-((4R)-2-(((2R,3R,4R,5R)-5-(2,6-diamino-9H-purin-9-yl)-3,4-dihydroxy-4-methyltetrahydrofuran-2-yl)methoxy)-5,5-dimethyl-2-oxido-1,3,2-dioxaphosphinane-4-carboxamido)propanoate, 30

To a stirred solution of 5 (0.083 mmol, 52.8 mg) at 0° C. was added dropwise a 1 M solution of t-BuMgCl (0.25 mmol, 0.25 mL). After 30 min stirring at 0° C., a 0.2M solution of 28 (0.41 mmol, 2.08 mL) in THF was added dropwise at room temperature. The solution was stirred 5 days at room temperature then evaporated to dryness. The residue was purified by silica gel column chromatography to remove the unreacted amount of 28 (60% EtOAc in hexanes, then 15% MeOH in CH₂Cl₂). The purified fraction was evaporated, dried under high-vacuum and diluted in CH₂Cl₂ (5 mL). Methanesulfonic acid (0.23 mmol, 14.1 μL) was added and the solution was heated to reflux for 5 h. The solution was neutralized by adding Et₃N (0.23 mmol, 30 μL) and evaporated to dryness. The residue was purified by silica gel column chromatography (up to 10% MeOH in CH₂Cl₂) to give 30 (0.03 mmol, 18.0 mg). ¹H-NMR (400 MHz, CD₃OD) δ ppm 1.00 (s, 3H), 1.11 (s, 3H), 1.15 (s, 3H), 1.22 (t, J=7.1 Hz, 3H), 2.53 (dt, J=6.7, 2.4 Hz, 2H), 3.53-3.36 (m, 2H), 4.09 (q, J=7.2 Hz, 2H), 4.33-4.13 (m, 4H), 4.55 (ddd, J=11.7, 7.2, 2.0 Hz, 1H), 4.68 (ddd, J=11.6, 6.6, 2.0 Hz, 1H), 4.75 (d, J=4.0 Hz, 1H), 5.98 (s, 1H), 7.90 (s, 1H); ³¹P-NMR (CD₃OD): −4.87; LC/MS calcd. for C₂₂H₃₅N₇O₁₀P 588.2, observed: 588.1 (M+1).

### Example 8

### Synthesis of 2′-F-2′-C-Me 2,6-Diamino Purine Monophosphate Prodrug 36

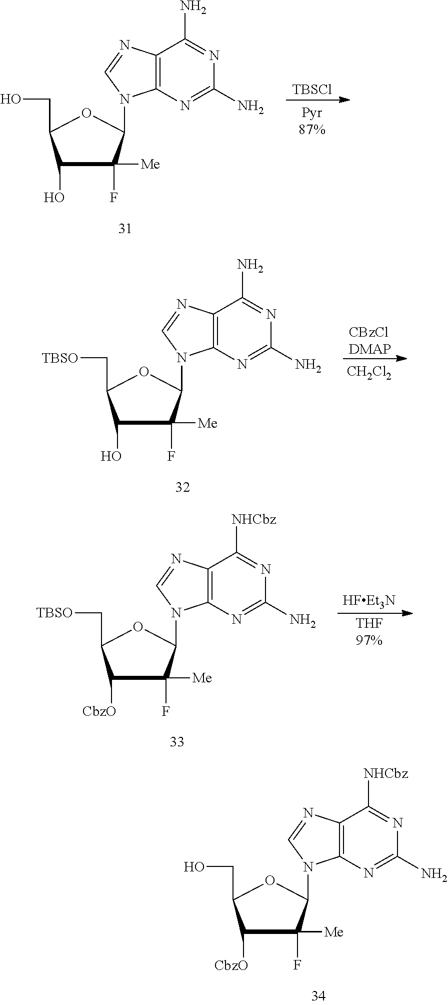

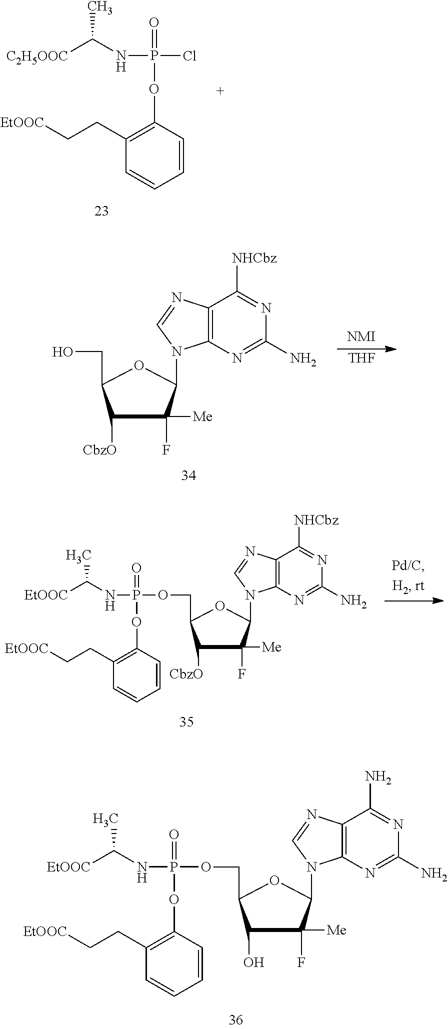

### (2R,3R,4R,5R)-5-(2,6-Diamino-9H-purin-9-yl)-4-fluoro-2-(hydroxymethyl)-4-methyltetrahydrofuran-3-ol, 31

¹H-NMR (CD₃OD): 1.18 (d, J=22.3 Hz, 3H), 3.87 (dd, J=13.0, 3.3 Hz, 1H), 4.02-4.06 (m, 2H), 4.40 (dd, J=24.4, 9.2 Hz, 1H), 6.12 (d, J=18.0 Hz, 1H), 8.13 (s, 1H);

¹³C-NMR (CD₃OD): 15.6, 15.8, 59.6, 71.2, 71.4, 82.3, 89.0, 89.4, 100.2, 102.0, 113.1, 136.5, 151.1, 156.5, 160.8. LC/MS calcd. for C₁₁H₁₅FN₆O₃ 298.1, observed: 299.2 (M+1).

### (2R,3R,4R,5R)-2-(((tert-Butyldimethylsilyl)oxy)methyl)-5-(2,6-diamino-9H-purin-9-yl)-4-fluoro-4-methyltetrahydrofuran-3-ol, 32

To a stirred solution of (2R,3R,4R,5R)-5-(2,6-diamino-9H-purin-9-yl)-4-fluoro-2-(hydroxymethyl)-4-methyltetrahydrofuran-3-ol, 31 (230 mg, 0.77 mmol) in pyridine was added TBDMSCl (256 mg, 1.69 mmol). The solution was stirred overnight and methanol (2 mL) was added. After stirring for 20 min the solution was evaporated to dryness and coevaporated two times with toluene. The residue was purified by silica gel column chromatography (0% to 3% MeOH in CH₂Cl₂) to afford compound 32 (275 mg, 0.67 mmol, 87%). ¹H-NMR (CD₃OD): 0.17 (s, 6H), 0.98 (s, 9H), 1.19 (d, J=22.2 Hz, 3H), 3.97 (dd, J=12.0, 2.5 Hz, 1H), 4.06 (dd, J=9.4, 1.3 Hz, 1H), 4.16 (dd, J=12.0, 1.7 Hz, 1H), 4.27 (dd, J=24.6, 9.4 Hz, 1H), 6.11 (d, J=16.7 Hz, 1H), 8.24 (s, 1H); ¹³C-NMR (CD₃OD): −5.276, −5.209, 16.7, 17.0, 19.5, 26.6, 62.3, 71.9, 72.1, 83.4, 89.4, 89.8, 101.4, 103.2, 113.9, 137.7, 152.7, 156.5, 160.6. LC/MS calcd. for C₁₇H₂₉FN₆O₃Si 412.2, observed: 413.3 (M+1).

### Benzyl (2-amino-9-((2R,3R,4R,5R)-4-(((benzyloxy)carbonyl)oxy)-5-(((tert-butyldimethylsilyl)oxy)methyl)-3-fluoro-3-methyltetrahydrofuran-2-yl)-9H-purin-6-yl)carbamate, 33

To a stirred solution of compound 32 (225 mg, 0.55 mmol) in CH₂Cl₂ (5 mL) at 0° C. were successively added DMAP (266 mg, 2.2 mmol) and CBzCl (0.31 mL, 2.18 mmol). After stirring at room temperature for 6 h, the solution was cooled to 0° C. and DMAP (266 mg, 2.2 mmol) and CBzCl (0.31 mL, 2.18 mmol) were added once again. After stirring overnight at room temperature, reaction was quenched with water and CH₂Cl₂ was added. The organic and aqueous layers were separated, and the organic layer was washed two more times with water. The combined organic layers were dried over Na₂SO₄, filtrated and evaporated. The residue was purified by silica gel column chromatography (10% to 45% EtOAc in hexanes) to afford compound 33 (300 mg, 0.44 mmol, 81%). ¹H-NMR (CD₃OD): 0.06 (d, J=4.1 Hz, 6H), 0.91 (s, 9H), 1.17 (d, J=22.4 Hz, 3H), 3.80 (dd, J=12.1, 2.6 Hz, 1H), 4.05 (dd, J=12.1, 2.1 Hz, 1H), 4.27 (d, J=9.1 Hz, 1H), 5.14-5.23 (m, 5H), 5.53 (dd, J=22.6, 9.1 Hz, 1H), 6.16 (d, J=16.7 Hz, 1H), 7.26-7.43 (m, 10H), 8.30 (s, 1H). ¹³C-NMR (CD₃OD): −5.4, 17.4, 17.6, 19.4, 26.5, 62.2, 68.3, 71.6, 75.5, 75.7, 81.2, 89.6, 90.0, 100.4, 102.2, 116.4, 129.2, 129.3, 129.5, 129.6, 129.7, 129.8, 136.5, 137.4, 138.9, 151.5, 153.3, 154.1, 155.9, 162.0; LC/MS calcd. for C₃₃H₄₁FN₆O₇Si 680.3, observed: 681.3 (M+1).

### Benzyl (2-amino-9-((2R,3R,4R,5R)-4-(((benzyloxy)carbonyl)oxy)-3-fluoro-5-(hydroxymethyl)-3-methyl tetrahydrofuran-2-yl)-9H-purin-6-yl)carbamate, 34

To a stirred solution of compound 33 (245 mg, 0.36 mmol) in THF (5 mL) at 0° C. was added Et₃N.3HF (0.234 mL, 1.44 mmol). After stirring 24 h at room temperature, the solution was neutralized with a saturated solution of NaHCO₃ then EtOAc was added. The organic and aqueous layers were separated, and the organic layer was washed once again with a saturated solution of NaHCO₃ and finally by water. The combined organic layers were dried over Na₂SO₄, filtrated and evaporated. The residue was purified by silica gel column chromatography (1% then 2% MeOH in CH₂Cl₂) to get compound 34 (198 mg, 0.35 mmol, 97%). ¹H-NMR (CD₃OD): 1.17 (d, J=22.6, 3H), 3.78 (dd, J=12.7, 3.2 Hz, 1H), 3.97 (dd, J=12.7, 2.4 Hz, 1H), 4.24 (d, J=9.0 Hz, 1H), 5.23 (s, 2H), 5.19 (s, 2H), 5.62 (dd, J=21.2, 9.0 Hz, 1H), 6.16 (d, J=18.0 Hz, 1H), 7.26-7.43 (m, 10H), 8.25 (s, 1H); ¹³C-NMR (CD₃OD): 17.6, 17.8, 60.8, 68.3, 71.5, 76.2, 76.4, 81.6, 90.2, 90.6, 100.3, 102.2, 103.0, 116.6, 129.3, 129.4 (2C), 129.6, 129.7 (2C), 136.6, 137.4, 139.8, 151.5, 153.4, 154.1, 155.9, 161.8; LC/MS calcd. for C₂₇H₂₇FN₆O₇ 566.2, observed: 567.2 (M+1).

### Ethyl 3-(2-(((S)-(((2R,3R,4R,5R)-5-(2-amino-6-(((benzyloxy)carbonyl)amino)-9H-purin-9-yl)-3-(((benzyloxy)carbonyl)oxy)-4-fluoro-4-methyltetrahydrofuran-2-yl)methoxy)(((S)-1-ethoxy-1-oxopropan-2-yl)amino)phosphoryl)oxy)phenyl)propanoate, 35

(2R)-Ethyl 2-(chloro(phenoxy)phosphorylamino)propanoate (0.5 M, 0.58 mL, 0.29 mmol) was added dropwise to a solution of 34 (32.8 mg, 0.057 mmol) and N-methylimidazole (23 μL, 0.29 mmol) in THF (0.1 mL) at 0° C. The resulting mixture was stirred overnight toward rt. After removed the solvent under reduced pressure, the residue was purified by flash column chromatography in a gradient of MeOH (gradient 0% to 10% MeOH in CH₂Cl₂) to afford 42 mg white solid 35 (80% yield). ¹H NMR (400 MHz, CD₃OD) δ 1.10-1.32 (m, 12H), 2.54-2.64 (m, 2H), 2.89-2.97 (m, 2H), 3.89-4.11 (m, 6H), 4.40-4.61 (m, 2H), 5.16-5.28 (m, 4H), 5.86-5.99 (m, 1H), 6.14-6.21 (m, 1H), 6.90-7.45 (m, 14H), 7.97 (s, 1H); ³¹PNMR (162 MHz, CD₃OD): 4.74, 4.77; LC/MS calcd. for C₄₃H₅₀FN₇O₁₃P 922.3, observed: 922.2 (M+1)⁺.

### Ethyl 3-(2-(((S)-(((2R,3R,4R,5R)-5-(2,6-diamino-9H-purin-9-yl)-4-fluoro-3-hydroxy-4-methyltetra-hydrofuran-2-yl)methoxy)(((S)-1-ethoxy-1-oxopropan-2-yl)amino)phosphoryl)oxy)phenyl)propanoate, 36

A mixture of 35 (42 mg) and 10 mg of 10% Pd/C in 5 mL of ethanol was charged with hydrogen atmosphere at room temperature and stirred overnight. The resulting suspension was degassed with a stream of nitrogen, filtered, the filtrate was concentrated and the residue was purified by silica gel column (gradient 0 to 10% MeOH in DCM) to afford 23 mg of prodrug 36 in 77% yield. ¹H NMR (400 MHz, CD₃OD) δ 1.14-1.34 (m, 12H), 2.59-2.64 (m, 2H), 2.96-3.00 (m, 2H), 3.93-4.19 (m, 6H), 4.47-4.62 (m, 3H), 6.08-6.15 (m, 1H), 7.07-7.37 (m, 4H), 7.85 (s, 1H); ³¹PNMR (162 MHz, CD₃OD): 4.88, 4.95; LC/MS calcd. for C₂₇H₃₈FN₇O₉P 653.2, observed: 653.3 (M+1)⁺.

### Example 9

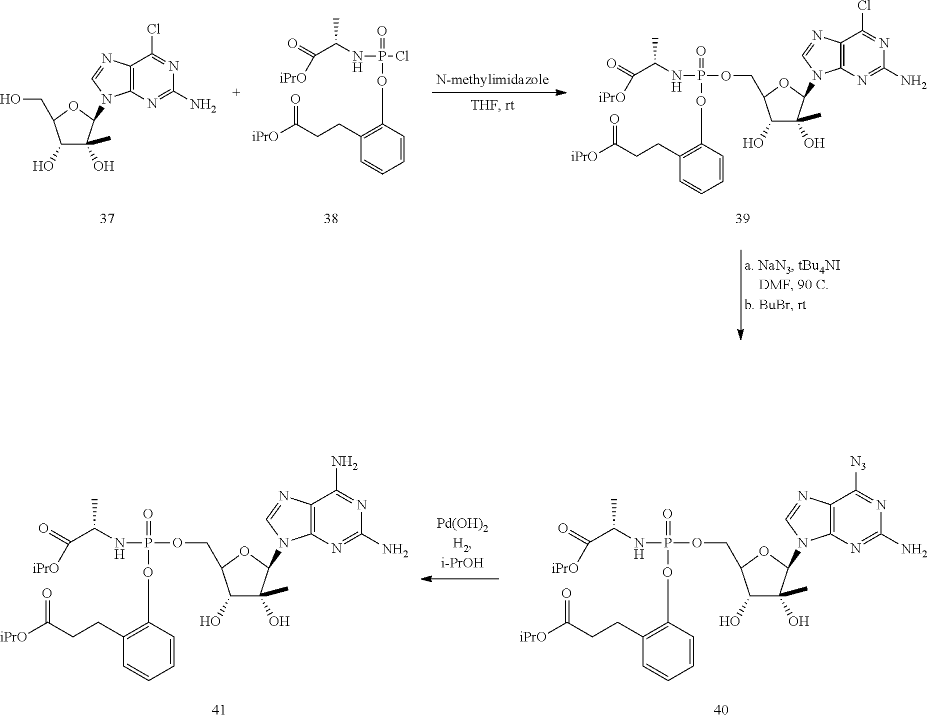 [SMILES: C[C@@]1(O)[C@H](O)C(CO)O[C@H]1n1cnc2c(Cl)nc(N)nc21 ; CC(C)OC(=O)CCc1ccccc1OP(=O)(Cl)N[C@@H](C)C(=O)OC(C)C ; CC(C)OC(=O)CCc1ccccc1OP(=O)(N[C@@H](C)C(=O)OC(C)C)OCC1O[C@@H](n2cnc3c(Cl)nc(N)nc32)[C@](C)(O)[C@@H]1O ; CC(C)OC(=O)CCc1ccccc1OP(=O)(N[C@@H](C)C(=O)OC(C)C)OCC1O[C@@H](n2cnc3c(N)nc(N)nc32)[C@](C)(O)[C@@H]1O ; CC(C)OC(=O)CCc1ccccc1OP(=O)(N[C@@H](C)C(=O)OC(C)C)OCC1O[C@@H](n2cnc3c(N=[N+]=[N-])nc(N)nc32)[C@](C)(O)[C@@H]1O]

### Isopropyl 3-(2-(((((3R,4R,5R)-5-(2-amino-6-chloro-9H-purin-9-yl)-3,4-dihydroxy-4-methyltetrahydro-furan-2-yl)methoxy)(((S)-1-isopropoxy-1-oxopropan-2-yl)amino)phosphoryl)oxy)phenyl)propanoate 3a

To a stirred solution of 37 (630 mg, 1.91 mmol) and 38 (2.4 g, 5.71 mmol) in anhydrous THF (10 mL) and MeCN (1 mL) was added NMI (445 μL, 5.71 mmol) at room temperature. The reaction mixture was stirred at rt for 2.5 h. The solvents were removed under reduced pressure and the residue was purified by silica gel column chromatography (0% to 8% MeOH in dichloromethane). 1.2 g of compound 39 was obtained (82% yield). LC/MS calcd. for C₂₉H₄₀ClN₆O₁₀P 698.2, observed: 699.2 (M+1)⁺.

### Isopropyl 3-(2-(((((2R,3R,4R,5R)-5-(2,6-diamino-9H-purin-9-yl)-3,4-dihydroxy-4-methyltetrahydro-furan-2-yl)methoxy)(((S)-1-isopropoxy-1-oxopropan-2-yl)amino)phosphoryl)oxy)phenyl)propanoate 41

A solution of 39 (1.2 g, 1.57 mmol), NaN₃ (155 mg, 2.36 mmol), ^{t}Bu₄NI (295 mg, 0.78 mmol) in DMF (2 mL) was stirred at 90° C. for 5 h. The reaction mixture was cooled to room temperature, n-BuBr (0.22 mL, 2 mmol) was added and stirred at rt for 1 h to convert excess NaN₃ to BuN₃. After removal of the solvent under reduced pressure, the residue was partitioned between EtOAc (100 mL) and water (30 mL). The separated water phase was extracted with EtOAc (3×30 mL) and the combined organic layer was dried over Na₂SO₄. After removal of the solvent, the residue was added Pd(OH)₂/C and iPrOH (15 mL). The mixture was charged with hydrogen (50 PSI) overnight for the completion of reduction reaction. The reaction mixture was filtered through a celite pad and the filtrate was concentrated under reduced pressure. The residue was portioned with EtOAc (100 mL) and water (20 mL). The water phase was extracted with EtOAc (3×30 mL) and the combined organic layer was dried over Na₂SO₄. After removed the solvent, the residue was purified by flash column chromatography (0% to 15% MeOH in CH₂Cl₂) to afford 650 mg of 41 as a white solid (61% yield; two steps). ¹H-NMR (CD₃OD) (1:1 mixture of P diastereomers): 0.97 (s, 3H, CH₃), 1.13-1.21 (m, 9H, 3×CH₃), 1.33 (s, 3H, CH₃), 2.56-2.62 (m, 2H, CH₂), 2.97-3.03 (m, 2H, CH₂), 3.91-3.95 (m, 1H), 4.18-4.26 (m, 2H), 4.88-4.61 (m, 2H), 4.83-4.97 (m, 2H), (m, 14H), 5.93 (s, 1H), 7.08-7.40 (m, 4H, Ar—H), 7.86 (s, 1H, H₈); ³¹PNMR (CD₃OD): 4.99, 5.09; LC/MS calcd. for C₂₉H₄₂N₇O₁₀P 679.3, observed: 680.3 (M+1)⁺.

### Example 10

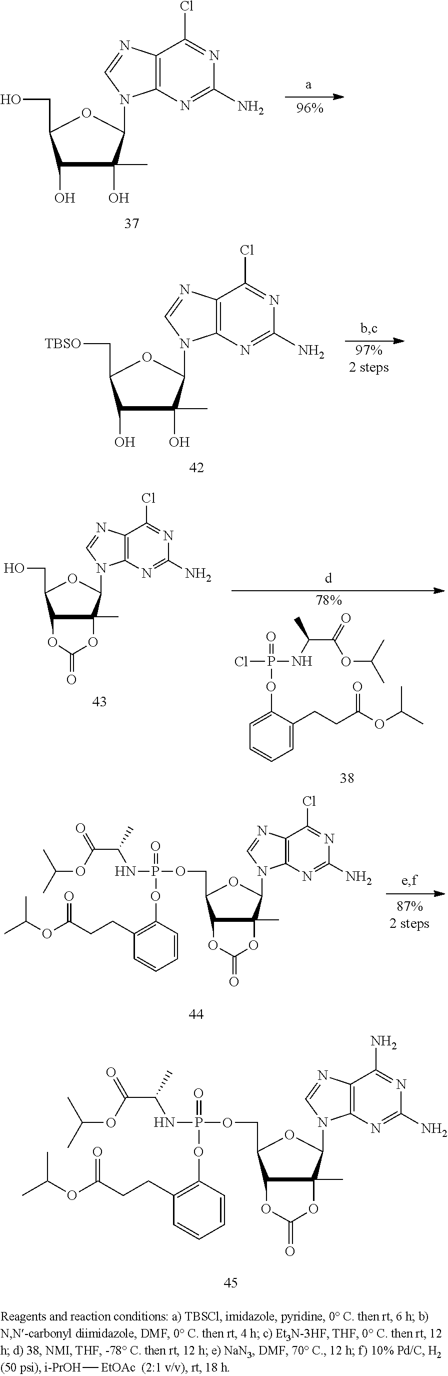

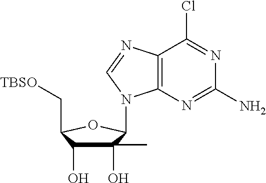

### (2R,3R,4R,5R)-2-(2-Amino-6-chloro-9H-purin-9-yl)-5-(((tert-butyldimethylsilyl)oxy)methyl)-3-methyltetrahydrofuran-3,4-diol (42)

To a solution of compound 37 (1.0 g, 3.20 mmol) in 20 mL of anhydrous pyridine was added imidazole (0.27 g, 4.0 mmol) and t-butyldimethylsilyl chloride (TBSCl) (0.72 g, 4.8 mmol) at 0° C. under a N₂ atmosphere. After stirring for 6 h, the solution was treated with MeOH (1.0 mL) at room temperature and concentrated under reduced pressure. The residue was purified by silica gel column chromatography (CH₂Cl₂ to CH₂Cl₂:MeOH; 10:1) to give compound 42 (1.32 g, 3.07 mmol) in 96% yield. MS-ESI⁺ m/z 430 (M+H⁺).

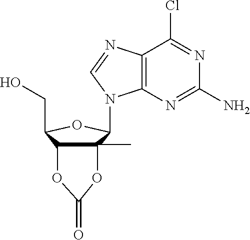

### (3aR,4R,6R,6aR)-4-(2-Amino-6-chloro-9H-purin-9-yl)-6-(hydroxymethyl)-3a-methyltetrahydrofuro[3,4-d][1,3]dioxol-2-one (43)

To a solution of compound 42 (0.89 g, 2.10 mmol) in 10 mL of anhydrous DMF was added N,N′-carbonyldiimidazole (0.85 g, 5.18 mmol) at 0° C. under a N₂ atmosphere. After stirring for 4 h, the reaction solution was concentrated under reduced pressure. The residue was purified by silica gel column chromatography (hexane:EtOAc; 4:1 to 1:2) to give 2′,3′-O,O-carbonate intermediate. To a solution of the 2′,3′-O,O-carbonate intermediate in 20 mL of THF was added Et₃N-3HF (1.65 mL, 10.20 mmol) at 0° C. under a N₂ atmosphere. After stirring for 12 h at room temperature, the resulting solution was concentrated under reduced pressure. The residue was purified by silica gel column chromatography (hexane:EtOAc; 10:1 to EtOAc:MeOH; 20:1) to give compound 43 (0.70 g, 2.04 mmol) in 97% yield (2 steps). ¹H NMR (400 MHz, DMSO-d₆) δ 8.34 (s, 1H), 7.12 (br, 2H), 6.37 (s, 1H), 5.34 (t, J=5.6 Hz, 1H), 5.08 (d, J=3.6 Hz, 1H), 4.40 (q, J=3.6 Hz, 1H), 3.82-3.70 (m, 2H), 1.30 (s, 3H); MS-ESI⁺ m/z 342 (M+H⁺).

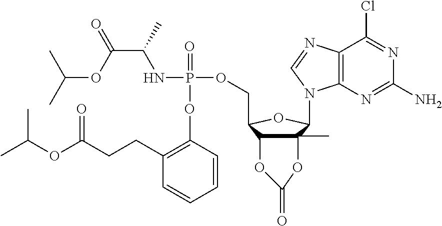

### Isopropyl 3-(2-(((((3aR,4R,6R,6aR)-6-(2-amino-6-chloro-9H-purin-9-yl)-6a-methyl-2-oxotetrahydrofuro[3,4-d][1,3]dioxol-4-yl)methoxy)(((S)-1-isopropoxy-1-oxopropan-2-yl)amino)phosphoryl)oxy)phenyl)propanoate (44)

To a solution of compound 43 (0.54 g, 1.58 mmol) in 10 mL of anhydrous THF was added a solution of phosphoramidate chloride 38 (1.66 g, 3.95 mmol) of 10 mL of THF and N-methylimidazole (0.65 g, 7.90 mmol) at −78° C. under a N₂ atmosphere. After stirring for 12 h at room temperature, the reaction solution was concentrated under reduced pressure. The residue was purified by silica gel column chromatography (hexane:EtOAc; 4:1 to 1:2) to give compound 44 (0.89 g, 1.23 mmol) in 78% yield. ¹H NMR (400 MHz, CDCl₃) δ 8.81-7.79 (s, 1H), 7.40-7.05 (m, 4H), 6.40-5.90 (br, 2H), 6.13-6.08 (s, 1H), 5.61 (d, J=4.8 Hz, 0.5H), 5.34 (d, J=4.4 Hz, 0.5H), 5.09-4.90 (m, 3H), 4.51-4.43 (m, 1H), 4.24-3.95 (m, 2H), 3.85-3.78 (m, 1H), 3.03-2.86 (m, 2H), 2.64-2.54 (m, 2H), 1.43-1.13 (m, 18H); MS-ESI⁺ m/z 725 (M+H⁺).

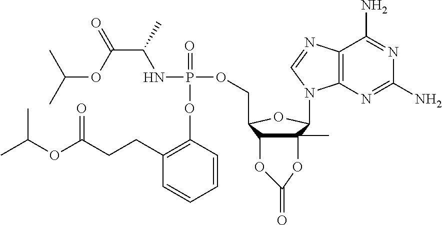

### Isopropyl 3-(2-(((((3aR,4R,6R,6aR)-6-(2,6-diamino-9H-purin-9-yl)-6a-methyl-2-oxotetrahydrofuro[3,4-d][1,3]dioxol-4-yl)methoxy)(((S)-1-isopropoxy-1-oxopropan-2-yl)amino)phosphoryl)oxy)phenyl)propanoate (45)

To a solution of compound 44 (0.47 g, 0.65 mmol) in 10 mL of anhydrous DMF was added NaN₃ (0.13 g, 1.95 mmol) at room temperature under a N₂ atmosphere. After stirring for 12 h at 70° C., the resulting solution was poured into 50 mL of EtOAc and washed with cold water (20 mL×3) and brine (20 mL). The organic layer was dried over Na₂SO₄ and concentrated under reduced pressure. To a solution of the residue in 15 mL of co-solvent i-PrOH:EtOAc; 2:1 was added 0.04 g of Pd/C (10% Pd on activated carbon). After shaking for 18 h under H₂ (50 psi), the N₂ degassed solution was treated with celite, stirred 30 min and filtered. The filtrate was purified by silica gel column chromatography (hexane:EtOAc; 1:5 to EtOAc:MeOH; 20:1) to give compound 45 (0.40 g, 0.57 mmol) in 87% yield (the ratio of diastereomers (Rₚ/Sₚ)=1:1 by ³¹P NMR). ¹H NMR (400 MHz, CD₃OD) δ 7.86-7.81 (s, 1H), 7.40-7.09 (m, 4H), 6.32-6.30 (s, 1H), 5.38-5.34 (m, 1H), 5.02-4.87 (m, 2H), 4.80-4.70 (m, 1H), 4.56-4.38 (m, 2H), 3.99-3.92 (m, 1H), 3.00-2.94 (m, 2H), 2.63-2.55 (m, 2H), 1.38-1.33 (m, 6H), 1.22-1.13 (m, 12H); ³¹P NMR (162 MHz, CDCl₃) δ 5.45, 5.25; MS-ESI⁺ m/z 706 (M+H⁺).

### Example 11

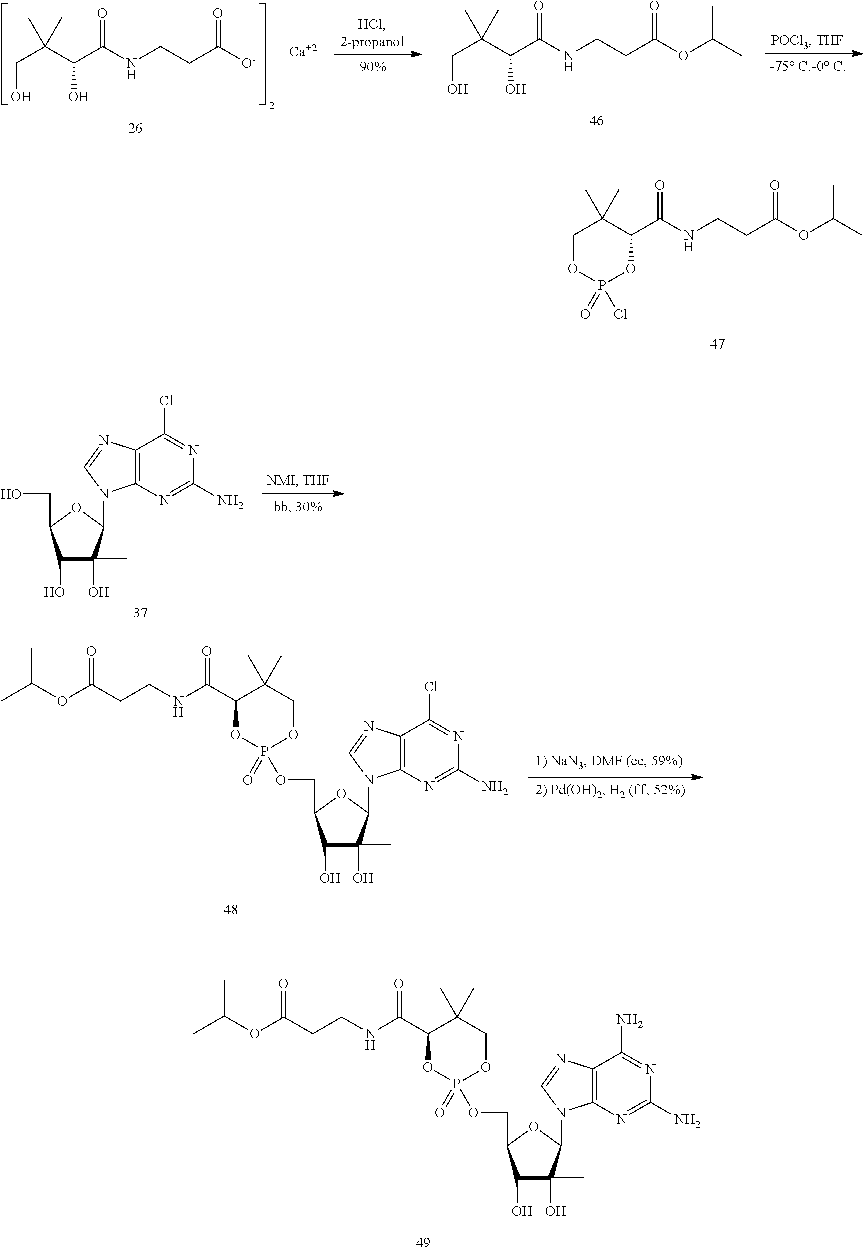

### (R)-Isopropyl 3-(2,4-dihydroxy-3,3-dimethylbutanamido)propanoate, 46

A suspension of panthenoate calcium 26 (10 g, 42 mmol) in 2-propanol (200 mL) was cooled to 0° C. and treated with HCl gas until a clear solution was obtained (ca. 15 min). The introduction of HCl gas was terminated, mixture was allowed to warm to room temperature and stirred overnight. Solvents were evaporated under reduced pressure and the resulting residue was dissolved in EtOAc and washed with NaHCO₃ (5%). The combined organic layers were dried over Na₂SO₄, filtered and evaporated to give 46 as a clear oil (10 g, 90%). ¹H-NMR (400 MHz, CDCl₃) δ ppm 0.86 (s, 3H), 0.97 (s, 3H), 1.22 (d, J=6.4 Hz, 6H), 2.51 (t, J=6.4 Hz, 2H), 3.55-3.45 (m, 4H), 3.98 (s, 1H), 3.98 (s, 1H), 5.00 (t, 6.4 Hz 1H), 7.33 (t, J=5.7 Hz, 1H). LC/MS calcd. For C₁₂H₂₄NO₅ 262.1, observed: 262.1 (M+1).

### Isopropyl 3-((4R)-2-chloro-5,5-dimethyl-2-oxido-1,3,2-dioxaphosphinane-4-carboxamido)propanoate, 47

To a solution of compound 46 (1 g, 3.8 mmol) in THF (15 mL) was added Et₃N (11.2 mmol, 1.6 mL) at 0° C. After 30 min stirring, this solution was gradually added to a cooled solution of POCl₃ (4.7 mmol, 0.45 mL) in THF (10 mL) at −75° C. The resulting solution was stirred for 1 h at 0° C. and another 30 min at room temperature. Solution was concentrated under reduced pressure, dissolved in dichloromethane (20 mL) and washed with NaHCO₃ (sat). The combined organic layers were dried over Na₂SO₄ and solvents were evaporated under reduced pressure. Compound 47 was dried under high vacuum and used as such without further purification. LC/MS calc. for C₁₂H₂₂ClNO₆P 342.0, observed: 342.0 (M+1).

### Isopropyl 3-((4R)-2-(((2R,3R,4R,5R)-5-(2-amino-6-chloro-9H-purin-9-yl)-3,4-dihydroxy-4-methyltetrahydrofuran-2-yl)methoxy)-5,5-dimethyl-2-oxido-1,3,2-dioxaphosphinane-4-carboxamido)propanoate, 48

To a solution of 2-amino-6-chloro-purine nucleoside 37 (0.2 g, 0.63 mmol) in THF (9 mL) was added N-methylimidazole (0.15 mL, 1.9 mmol) at room temperature. After stirring for 45 min, the solution was cooled to 0° C. and a solution of 47 (5 mL, 0.5 M in THF) was added dropwise. The reaction mixture was allowed to warm to room temperature and stirred overnight. Solvents were evaporated under reduced pressure and crude residue was purified by flash chromatography (eluent: 5% to 15% MeOH in CH₂Cl₂). Compound 48 was obtained (118 mg, 0.19 mmol) in 30% yield. LC/MS calcd. for C₂₃H₃₅ClN₆O₁₀P 621.1, observed 621.1 (M+1).

### Isopropyl 3-((4R)-2-(((2R,3R,4R,5R)-5-(2,6-diamino-9H-purin-9-yl)-3,4-dihydroxy-4-methyltetrahydrofuran-2-yl)methoxy)-5,5-dimethyl-2-oxido-1,3,2-dioxaphosphinane-4-carboxamido)propanoate, 49

A solution of 48 (100 mg, 0.16 mmol) and NaN₃ (52 mg, 0.8 mmol) in DMF (3 mL) was heated to 80° C. and stirred for 5 h (reaction progress was monitored by LC-MS). Upon completion of the reaction, the mixture was concentrated under reduced pressure and the crude residue was purified by flash chromatography (0% to 20% MeOH in CH₂Cl₂). The 6-azido compound was obtained in pure form as a white solid (60 mg, 0.095 mmol) in 59% yield. LC-MS calcd. for C₂₃H₃₄N₉O₁₀P 627.2, observed 628.2 (M+1).

The 6-azido compound from above (60 mg, 0.095 mmol) and a catalytic amount of Pd(OH)₂/C in ethylacetate (3 mL), was subjected to hydrogenation under atmospheric pressure at room temperature for 8 h. The N₂ sparged mixture was filtered through a pad of celite and the resulting celite was washed with a 50% solution of CH₂Cl₂ and CH₃OH. Solvents were evaporated under reduced pressure and the crude reside was purified by preparative TLC plate (eluent: 15% MeOH in CH₂Cl₂). Compound 49 was obtained as mixture of diastereomers (30 mg, 52%). ¹³P-NMR (CD₃OD): −4.84, −7.21; LC-MS calcd. for C₂₃H₃₇N₇O₁₀P 602.2, observed 602.2 (M+1).

### Example 12

### NS5B Enzyme Assay

The 21-amino-acid C-terminal truncated HCV NS5B RNA polymerase was cloned from the HCV replicon cells, modified with a six-His-terminal tail, expressed in a prokaryotic expression vector (pQE60; Qiagen), and subsequently purified over a Talon cobalt affinity resin column (Clontech, Palo Alto, Calif.).¹ Purification was monitored by SDS-PAGE and Western blotting. The resulting purified protein was dialyzed overnight against 50 mM sodium phosphate (pH 8.0)—300 mM sodium chloride—0.5% Triton X-100—50% glycerol—2 mM dithiothreitol. The dialysate maintained consistent activity for more than 6 months when stored at −20° C. Protein was quantified with the Coomassie Plus protein assay reagent (Pierce) by using a bovine serum albumin standard from the same supplier.

NS5B RNA polymerase reaction was studied by monitoring the incorporation of ³²P-labeled UMP into the newly synthesized RNA strand by using minus IRES as the template. A steady-state reaction was performed in a total volume of 140 mL containing 2.8 mg of minus IRES RNA template, 140 units of anti-RNase (Ambion), 1.4 mg of NS5B, an appropriate amount of [a-³²P]UTP, various concentrations of natural and modified nucleotides, 1 mM MgCl₂, 0.75 mM MnCl₂, and 2 mM dithiothreitol in 50 mM HEPES buffer (pH 7.5). The nucleotide concentration was changed depending on the inhibitor. The reaction temperature was 27° C. At the desired times, 20-mL aliquots were taken and the reaction was quenched by mixing the reaction mixture with 80 mL of stop solution containing 12.5 mM EDTA, 2.25 M NaCl, and 225 mM sodium citrate. In order to determine steady-state parameters for a natural nucleotide TP (NTP) substrate, one NTP concentration was varied and the concentrations of the other three NTPs were fixed at saturating concentrations. For determination of the Kᵢ for an A analog, the concentrations of UTP, GTP, and CTP were fixed at 10, 100, and 100 mM, respectively, and the concentrations of ATP and the A analog were varied. The radioactive RNA products were separated from unreacted substrates by passing the quenched reaction mixture through a Hybond N+ membrane (Amersham Biosciences) by using a dot blot apparatus. The RNA products were retained on the membrane and the free nucleotides were washed out. The membrane was washed four times with a solution containing 0.6 M NaCl and 60 mM sodium citrate. After the membrane was rinsed with water followed by rinsing with ethanol, the dots were cut out and the radioactivity was counted in a Packard liquid scintillation counter. The amount of product was calculated on the basis of the total radioactivity in the reaction mixture. The rate of the reaction was determined from the slope of the time course of product formation. To determine the inhibition constant (Kᵢ), reaction rates were determined with different concentrations of the substrate and the inhibitor and were fit to a competitive inhibition equation: ν=(Vₘₐₓ·[s])/{Kₘ·(1+[I]/Kᵢ)+[S]}, where ν is the observed rate, [S] is the substrate concentration, [I] is the inhibitor concentration, and Vₘₐₓ is the maximum rate. Kₘ is the Michaelis constant, and Kᵢ is the inhibition constant.

### REFERENCES

1) Stuyver L J, Whitaker T, McBrayer T R, Hernandez-Santiago B I, Lostia S, Tharnish P M, Ramesh M, Chu C K, Jordan R, Shi J, Rachakonda S, Watanabe K A, Otto M J, Schinazi R F. Ribonucleoside Analogue That Blocks Replication of Bovine Viral Diarrhea and Hepatitis C Viruses in Culture Antimicrob. Agents Chemother. 2003, 47, 244.

### Example 13

### RNA Synthesis and Chain Termination

i) Expression and Purification of HCV NS5B:

The HCV NS5B sequence, inserted into the expression vector pET-22 (Novagen), was expressed as a C terminally truncated enzyme (Δ21) in Escherichia coli BL21(DE3) and purified utilizing metal ion affinity chromatography (Talon kit from Clonetech). Sequences were confirmed by sequencing (Sequetech).

ii) Standard Reaction Conditions:

Reaction mixtures consisted of 1 μM RNA template (RNA20), 1.5 μM HCV NS5B, and 0.25 μM radiolabeled primer (P16) in a buffer containing 40 mM HEPES, pH 8, 10 mM NaCl, 1 mM dithiothreitol, and 0.2 mM MnCl₂. In addition, reactions contained 10 μM GTP-UTP and 3 μM test analog-TP. Reactions were stopped after 30 minutes and products were precipitated with isopropanol, heat denatured for 5 minutes at 95° C., and separated on 12% polyacrylamide, 7 M urea gels. The concentration of chain terminator required to inhibit 50% of full-length product formation (EC₅₀) was determined for a single site of nucleotide analog incorporation with template/primer.

iii) Data Acquisition and Analysis:

Gels were scanned and analyzed with a phosphorimager (FLA-7000, Fujifilm), and EC₅₀ values were calculated

FIG. 4 shows the incorporation of ((2R,3S,4R,5R)-5-(2,6-diamino-9H-purin-9-yl)-3,4-dihydroxytetrahydrofuran-2-yl)methyl tetrahydrogen triphosphate by HCV NS5B.

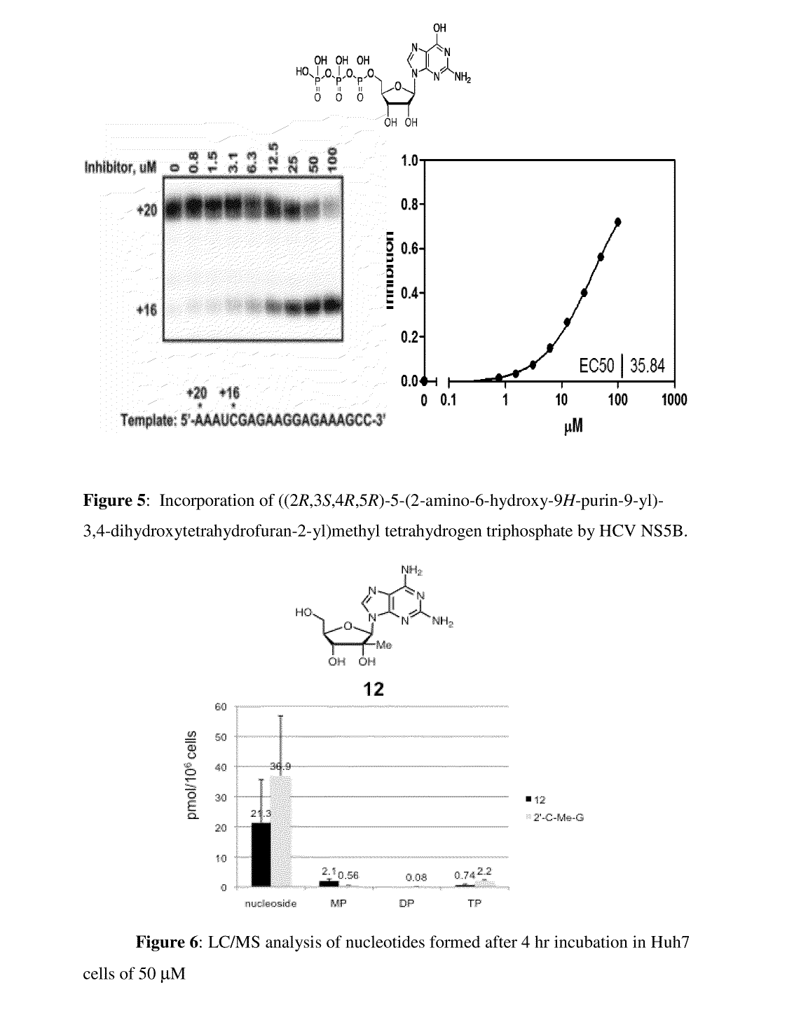

FIG. 5 shows the incorporation of ((2R,3S,4R,5R)-5-(2-amino-6-hydroxy-9H-purin-9-yl)-3,4-dihydroxytetrahydrofuran-2-yl)methyl tetrahydrogen triphosphate by HCV NS5B.

### Example 14

### Mitochondrial Toxicity Assays in HepG2 Cells

i) Effect of 2,6-diamino Purine Nucleoside Monophosphate Prodrugs on Cell Growth and Lactic Acid Production:

The effect on the growth of HepG2 cells was determined by incubating cells in the presence of 0 μM, 0.1 μM, 1 μM, 10 μM and 100 μM drug. Cells (5×10⁴ per well) were plated into 12-well cell culture clusters in minimum essential medium with nonessential amino acids supplemented with 10% fetal bovine serum, 1% sodium pyruvate, and 1% penicillin/streptomycin and incubated for 4 days at 37° C. At the end of the incubation period the cell number was determined using a hemocytometer. Also taught by Pan-Zhou X-R, Cui L, Zhou X-J, Sommadossi J-P, Darley-Usmer V M. “Differential effects of antiretroviral nucleoside analogs on mitochondrial function in HepG2 cells” Antimicrob. Agents Chemother. 2000; 44: 496-503. To measure the effects of the nucleoside analogs on lactic acid production, HepG2 cells from a stock culture were diluted and plated in 12-well culture plates at 2.5×10⁴ cells per well. Various concentrations (0 μM, 0.1 μM, 1 μM, 10 μM and 100 μM) of nucleoside analog were added, and the cultures were incubated at 37° C. in a humidified 5% CO₂ atmosphere for 4 days. At day 4 the number of cells in each well was determined and the culture medium collected. The culture medium was filtered, and the lactic acid content in the medium determined using a colorimetric lactic acid assay (Sigma-Aldrich). Since lactic acid product can be considered a marker for impaired mitochondrial function, elevated levels of lactic acid production detected in cells grown in the presence of 2,6-diamino 2′-C-Me purine nucleoside monophosphate prodrug analogs would indicate a drug-induced cytotoxic effect.

ii) Effect of 2,6-Diamino Purine Nucleoside Monophosphate Prodrugs on Mitochondrial DNA Synthesis:

A real-time PCR assay to accurately quantify mitochondrial DNA content has been developed (see Stuyver L J, Lostia S, Adams M, Mathew J S, Pai B S, Grier J, Tharnish P M, Choi Y, Chong Y, Choo H, Chu C K, Otto M J, Schinazi R F. Antiviral activities and cellular toxicities of modified 2′,3′-dideoxy-2′,3′-didehydrocytidine analogs. Antimicrob. Agents Chemother. 2002; 46: 3854-60). This assay was used in all studies described in this application that determine the effect of nucleoside analogs on mitochondrial DNA content. In this assay, low-passage-number HepG2 cells were seeded at 5,000 cells/well in collagen-coated 96-well plates. Nucleoside monophosphate analogs were added to the medium to obtain final concentrations of 0 μM, 0.1 μM, 10 μM and 100 μM. On culture day 7, cellular nucleic acids were prepared by using commercially available columns (RNeasy 96 kit; Qiagen). These kits co-purify RNA and DNA, and hence, total nucleic acids were eluted from the columns. The mitochondrial cytochrome c oxidase subunit II (COXII) gene and the β-actin or rRNA gene were amplified from 5 μl of the eluted nucleic acids using a multiplex Q-PCR protocol with suitable primers and probes for both target and reference amplifications. For COXII the following sense, probe and antisense primers are used, respectively: 5′-TGCCCGCCATCATCCTA-3′,5′-tetrachloro-6-carboxyfluorescein-TCCTCATCGCCCTCCCATCCC-TAMRA-3′ and 5′-CGTCTGTTATGTAAAGGATGCGT-3′. For exon 3 of the β-actin gene (GenBank accession number E01094) the sense, probe, and antisense primers are 5′-GCGCGGCTACAGCTTCA-3′,5′-6-FAMCACCACGGCCGAGCGGGATAMRA-3′ and 5′-TCTCCTTAATGTCACGCACGAT-3′, respectively. The primers and probes for the rRNA gene are commercially available from Applied Biosystems. Since equal amplification efficiencies were obtained for all genes, the comparative CT method was used to investigate potential inhibition of mitochondrial DNA synthesis. The comparative CT method uses arithmetic formulas in which the amount of target (COXII gene) is normalized to the amount of an endogenous reference (the β-actin or rRNA gene) and is relative to a calibrator (a control with no drug at day 7). The arithmetic formula for this approach is given by 2-ΔΔCT, where ΔΔCT is (CT for average target test sample−CT for target control)−(CT for average reference test−CT for reference control) (see Johnson M R, K Wang, J B Smith, M J Heslin, R B Diasio. Quantitation of dihydropyrimidine dehydrogenase expression by real-time reverse transcription polymerase chain reaction. Anal. Biochem. 2000; 278:175-184). A decrease in mitochondrial DNA content in cells grown in the presence of drug would indicate mitochondrial toxicity.

iii) Electron Microscopic Morphologic Evaluation:

NRTI induced toxicity has been shown to cause morphological changes in mitochondria (e.g., loss of cristae, matrix dissolution and swelling, and lipid droplet formation) that can be observed with ultrastructural analysis using transmission electron microscopy (see Cui L, Schinazi R F, Gosselin G, Imbach J L. Chu C K, Rando R F, Revankar G R, Sommadossi J P. Effect of enantiomeric and racemic nucleoside analogs on mitochondrial functions in HepG2 cells. Biochem. Pharmacol. 1996, 52, 1577-1584; Lewis W, Levine E S, Griniuviene B, Tankersley K O, Colacino J M, Sommadossi J P, Watanabe K A, Perrino F W. Fialuridine and its metabolites inhibit DNA polymerase gamma at sites of multiple adjacent analog incorporation, decrease mtDNA abundance, and cause mitochondrial structural defects in cultured hepatoblasts. Proc Natl Acad Sci USA. 1996; 93: 3592-7; Pan-Zhou X R, L Cui, X J Zhou, J P Sommadossi, V M Darley-Usmar. Differential effects of antiretroviral nucleoside analogs on mitochondrial function in HepG2 cells. Antimicrob. Agents Chemother. 2000, 44, 496-503). For example, electron micrographs of HepG2 cells incubated with 10 μM fialuridine (FIAU; 1,2′-deoxy-2′-fluoro-1-D-arabinofuranosly-5-iodo-uracil) showed the presence of enlarged mitochondria with morphological changes consistent with mitochondrial dysfunction. To determine if 2,6-diamino 2′-C-Me purine nucleoside monophosphate prodrugs would promote morphological changes in mitochondria, HepG2 cells (2.5×10⁴ cells/mL) would be seeded into tissue cultures dishes (35 by 10 mm) in the presence of 0 μM, 0.1 μM, 1 μM, 10 μM and 100 μM nucleoside analog. At day 8, the cells would be fixed, dehydrated, and embedded in Eponas described previously. Thin sections would be prepared, stained with uranyl acetate and lead citrate, and then examined using transmission electron microscopy.

The effect of compounds 8b-up, 12, and 8a on nuclear or mitochondrial DNA, or lactic acid production, in HepG2 Hepatoma cells was analyzed over a 14 day period. The procedure outlined in section (i) above was used for this analysis. The results are tabulated below:

| | Concentration | % Inhibition | Lactic acid levels |
|---|---|---|---|
| Compound | μM | MtDNA/nuclear DNA | (% of control) ± SD |
| 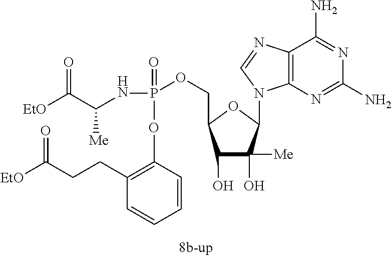 | 1 10 50 | 24/30 34/16 43/10 | 150 ± 1.7 120 ± 5.6 110 ± 1.9 |
| 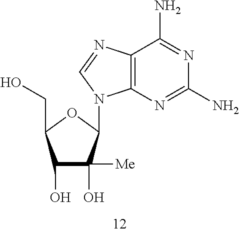 | 1 10 50 | <1/22 <1/46 <1/21 | 110 ± 0.9 170 ± 3.7 110 ± 6.1 |
| 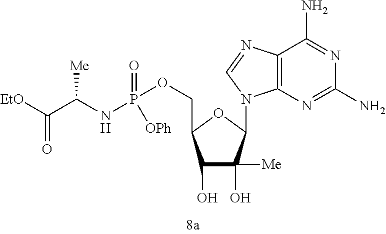 | 1 10 50 | <1/4.0 5.4/20 <1/<1 | 100 ± 13 140 ± 2.0 90 ± 4.5 |
| 3TC - control | 10 | <1/<1 | 91 ± 2.5 |
| ddC - control | 10 | 100/72 | 220 ± 11 |

Values in red represent³ 50% inhibition of total DNA levels (toxic for standard assay) or increased levels of lactic acid.

As shown in the table, 8b-up, 12, and 8a exhibited no significant effect on nuclear or mitochondrial DNA or lactic acid production up to 50 μM (in HepG2 Hepatoma cells, 14 day assay).

### Example 15

### Mitochondrial Toxicity Assays in Neuro2A Cells

To estimate the potential of nucleoside analogs to cause neuronal toxicity, mouse Neuro2A cells (American Type Culture Collection 131) would be used as a model system (see Ray A S, Hernandez-Santiago B I, Mathew J S, Murakami E, Bozeman C, Xie M Y, Dutschman G E, Gullen E, Yang Z, Hurwitz S, Cheng Y C, Chu C K, McClure H, Schinazi R F, Anderson K S. Mechanism of anti-human immunodeficiency virus activity of beta-D-6-cyclopropylamino-2′,3′-didehydro-2′,3′-dideoxyguanosine. Antimicrob. Agents Chemother. 2005, 49, 1994-2001). The concentrations necessary to inhibit cell growth by 50% (CC₅₀) would be measured using the 3-(4,5-dimethyl-thiazol-2-yl)-2,5-diphenyltetrazolium bromide dye-based assay, as described. Perturbations in cellular lactic acid and mitochondrial DNA levels at defined concentrations of drug would be carried out as described above. In all experiments, ddC and AZT would be used as control nucleoside analogs.

### Example 16

### Effect of Nucleotide Analogs on the DNA Polymerase and Exonuclease Activities of Mitochondrial DNA Polymerase γ

i) Purification of Human Polymerase γ:

The recombinant large and small subunits of polymerase γ would be purified as described previously (see Graves S W, Johnson A A, Johnson K A. Expression, purification, and initial kinetic characterization of the large subunit of the human mitochondrial DNA polymerase. Biochemistry. 1998, 37, 6050-8; Johnson A A, Tsai Y, Graves S W, Johnson K A. Human mitochondrial DNA polymerase holoenzyme: reconstitution and characterization. Biochemistry 2000; 39: 1702-8). The protein concentration was determined spectrophotometrically at 280 nm, with extinction coefficients of 234,420, and 71,894 M-1 cm-1 for the large and the small subunits of polymerase γ, respectively.

ii) Kinetic Analyses of Nucleotide Incorporation:

Pre-steady-state kinetic analyses were carried out to determine the catalytic efficiency of incorporation (k/K) for DNA polymerase γ for nucleoside-TP and natural dNTP substrates. This allowed determination of the relative ability of this enzyme to incorporate modified analogs and predict toxicity. Pre-steady-state kinetic analyses of incorporation of nucleotide analogs by DNA polymerase γ would be carried out essentially as described previously (see Murakami E, Ray A S, Schinazi R F, Anderson K S. Investigating the effects of stereochemistry on incorporation and removal of 5-fluorocytidine analogs by mitochondrial DNA polymerase gamma: comparison of D- and L-D4FC-TP. Antiviral Res. 2004, 62, 57-64; Feng J Y, Murakami E, Zorca S M, Johnson A A, Johnson K A, Schinazi R F, Furman P A, Anderson K S. Relationship between antiviral activity and host toxicity: comparison of the incorporation efficiencies of 2′,3′-dideoxy-5-fluoro-3′-thiacytidine-triphosphate analogs by human immunodeficiency virus type 1 reverse transcriptase and human mitochondrial DNA polymerase. Antimicrob Agents Chemother. 2004, 48, 1300-6). Briefly, a pre-incubated mixture of large (250 nM) and small (1.25 mM) subunits of polymerase γ and 6 0 nM DNA template/primer in 50 mM Tris-HCl, 100 mM NaCl, pH 7.8, was added to a solution containing MgCl₂ (2.5 mM) and various concentrations of nucleotide analogs. Reactions would be quenched and analyzed as described previously. Data would be fit to the same equations as described above.

iii) Assay for Human Polymerase γ 3′ 5′ Exonuclease Activity:

The human polymerase γ exonuclease activity was studied by measuring the rate of formation of the cleavage products in the absence of dNTP. The reaction was initiated by adding MgCl₂ (2.5 mM) to a pre-incubated mixture of polymerase γ large subunit (40 nM), small subunit (270 nM), and 1,500 nM chain-terminated template/primer in 50 mM Tris-HCl, 100 mM NaCl, pH 7.8, and quenched with 0.3M EDTA at the designated time points. All reaction mixtures would be analyzed on 20% denaturing polyacrylamide sequencing gels (8M urea), imaged on a Bio-Rad GS-525 molecular image system, and quantified with Molecular Analyst (Bio-Rad). Products formed from the early time points would be plotted as a function of time. Data would be fitted by linear regression with Sigma Plot (Jandel Scientific). The slope of the line was divided by the active enzyme concentration in the reaction to calculate the kexo for exonuclease activity (see Murakami E, Ray A S, Schinazi R F, Anderson K S. Investigating the effects of stereochemistry on incorporation and removal of 5-fluorocytidine analogs by mitochondrial DNA polymerase gamma: comparison of D- and L-D4FC-TP. Antiviral Res. 2004; 62: 57-64; Feng J Y, Murakami E, Zorca S M, Johnson A A, Johnson K A, Schinazi R F, Furman P A, Anderson K S. Relationship between antiviral activity and host toxicity: comparison of the incorporation efficiencies of 2′,3′-dideoxy-5-fluoro-3′-thiacytidine-triphosphate analogs by human immunodeficiency virus type 1 reverse transcriptase and human mitochondrial DNA polymerase. Antimicrob Agents Chemother. 2004; 48: 1300-6).

### Example 17

### Assay for Bone Marrow Cytotoxicity

Primary human bone marrow mononuclear cells would be obtained commercially from Cambrex Bioscience (Walkersville, Md.). CFU-GM assays would be carried out using a bilayer soft agar in the presence of 50 units/mL human recombinant granulocyte/macrophage colony-stimulating factor, while BFU-E assays used a methylcellulose matrix containing 1 unit/mL erythropoietin (see Sommadossi J P, Carlisle R. Toxicity of 3′-azido-3′-deoxythymidine and 9-(1,3-dihydroxy-2-propoxymethyl) guanine for normal human hepatopoietic progenitor cells in vitro. Antimicrob. Agents Chemother. 1987; 31: 452-454; Sommadossi, J P, Schinazi, R F, Chu, C K, and Xie, M Y. Comparison of Cytotoxicity of the (−) and (+) enantiomer of 2′,3′-dideoxy-3′-thiacytidine in normal human bone marrow progenitor cells. Biochem. Pharmacol. 1992; 44:1921-1925). Each experiment was performed in duplicate in cells from three different donors. AZT was used as a positive control. Cells would be incubated in the presence of the compound for 14-18 days at 37° C. with 5% CO₂, and colonies of greater than 50 cells are counted using an inverted microscope to determine IC₅₀. The 50% inhibitory concentration (IC₅₀) was obtained by least-squares linear regression analysis of the logarithm of drug concentration versus BFU-E survival fractions. Statistical analysis was performed with Student's t test for independent non-paired samples.

### Example 18

### Cytotoxicity Assay

The toxicity of the compounds was assessed in Vero, human PBM, CEM (human lymphoblastoid), and HepG2 cells, as described previously (see Schinazi R. F., Sommadossi J.-P., Saalmann V., Cannon D. L., Xie M.-Y., Hart G. C., Smith G. A. & Hahn E. F. Antimicrob. Agents Chemother. 1990, 34, 1061-67). Cycloheximide was included as positive cytotoxic control, and untreated cells exposed to solvent would be included as negative controls. The cytotoxicity IC₅₀ was obtained from the concentration-response curve using the median effective method described previously (see Chou T.-C. & Talalay P. Adv. Enzyme Regul. 1984, 22, 27-55; Belen'kii M. S. & Schinazi R. F. Antiviral Res. 1994, 25, 1-11). The data is tabulated below in Table 2:

TABLE 2
Cytotoxicity Data.
Cytotoxicity CC₅₀ (μM)

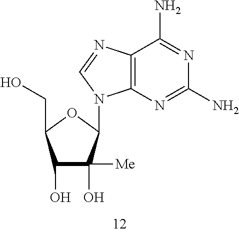

PBM > 100
CEM > 100
Vero > 100

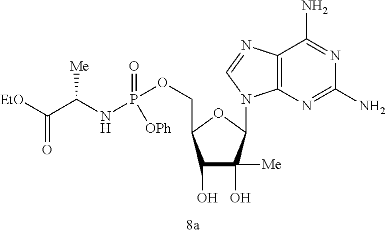

PBM > 100
CEM > 100
Vero > 100

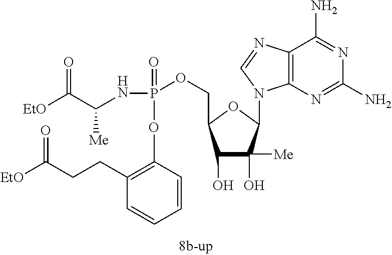

PBM > 100
CEM > 100
Vero > 100

### Example 19

### Adenosine Deaminase Assay

To determine the propensity for deamination of the nucleosides and monophosphate prodrugs by adenosine deaminase, nucleoside analogues can be incubated with the commercially available purified enzyme, and the reaction can be followed spectrophotometrically. Typical reaction conditions involve preparing a solution containing 50 μM nucleoside analog in 0.5 mL 50 mM potassium phosphate (pH 7.4) at 25° C. The typical reaction time is 7 minutes with 0.002 units of enzyme, and 120 minutes with 0.2 units of enzyme. (The unit definition of adenosine deaminase is one unit will deaminate 1.0 μmol of adenosine to inosine per minute at pH 7.5 at 25° C.) Deoxyadenosine is typically used as a positive control. Deoxyadenosine is 59% deaminated under the given conditions in 7 minutes with 0.002 units of enzyme. Deoxyguanosine is typically used as a negative control. Optical density can be measured at 265 nm or 285 nm. The difference in optical density between the beginning and the end of the experiment is divided by the extinction coefficient, and then multiplied by the volume of the reaction to determine the number of mols of substrate transformed into product. Mols of product would be divided by mols of substrate equivalent to a 100% complete reaction then multiplied by 100 to obtain percent deamination. The limit of detection is typically 0.001 optical density units.

### Example 20

### Synthesis of Nucleoside Analog Triphosphates

Nucleoside analog triphosphates were synthesized from the corresponding nucleosides, using the Ludwig and Eckstein's method. (Ludwig J, Eckstein F. “Rapid and efficient synthesis of nucleoside 5′-O-(1-thiotriphosphates), 5′-triphosphates and 2′,3′-cyclophosphorothioates using 2-chloro-4H-1,3,2-benzodioxaphosphorin-4-one” J. Org. Chem. 1989, 54 631-5) The crude nucleoside analog triphosphate can be purified, for example, by FPLC using a HiLoad 26/10 Q Sepharose Fast Flow Pharmacia column and gradient of TEAB buffer (pH 7.0). The product can be characterized by UV spectroscopy, proton and phosphorus NMR, mass spectroscopy and/or HPLC.

The resulting triphosphates can be used as controls for the cellular pharmacology assays described above and for kinetic work with HCV-Pol (for example, 2,6-diamino 2′-C-Me purine nucleoside triphosphate with HCV-Pol).

### Example 21

### HCV Replicon Assay¹

Huh 7 Clone B cells containing HCV Replicon RNA were seeded in a 96-well plate at 5000 cells/well, and the compounds tested at 10 μM in triplicate immediately after seeding. Following five days incubation (37° C., 5% CO₂), total cellular RNA was isolated using the versaGene RNA purification kit from Gentra. Replicon RNA and an internal control (TaqMan rRNA control reagents, Applied Biosystems) were amplified in a single step multiplex Real Time RT-PCR Assay. The antiviral effectiveness of the compounds was calculated by subtracting the threshold RT-PCR cycle of the test compound from the threshold RT-PCR cycle of the no-drug control (ΔCt HCV). A ΔCt of 3.3 equals a 1-log reduction (equal to 90% less starting material) in Replicon RNA levels. The cytotoxicity of the compounds was also calculated using the ΔCt rRNA values. (2′-C-Me-C) was used as the control. To determine EC₉₀ and IC₅₀ values², ΔCt: values were first converted into a fraction of the starting material,³ and then were used to calculate the % inhibition. The data on three compounds (Compound 12, Compound 8a, and compound 8b-up) is shown below in Table 3.

TABLE 3
HCV replicon data.
HCV Replicon (Huh7) (μM)

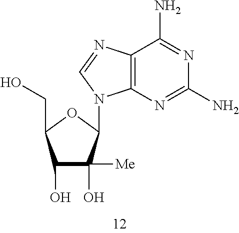

EC₅₀ = 3.7 +/− 1.8
EC₉₀ = 11 +/− 2.0
CC₅₀ > 10

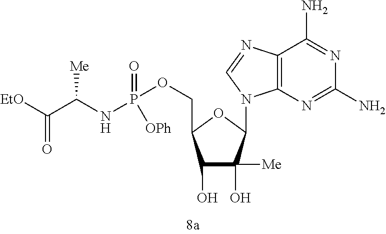

EC₅₀ = 1.3 +/− 0.6
EC₉₀ = 4.9 +/− 2.4
CC₅₀ > 10

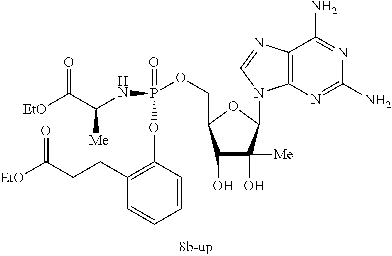

EC₅₀ = 0.2 +/− 0.1
EC₉₀ = 0.7 +/− 0.3
CC₅₀ > 10

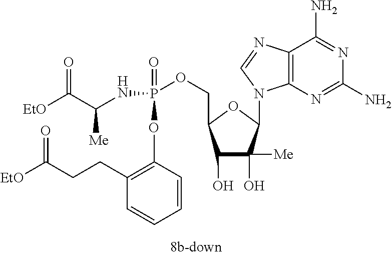

EC₅₀ = 2.3
EC₉₀ = 7.7
CC₅₀ > 3

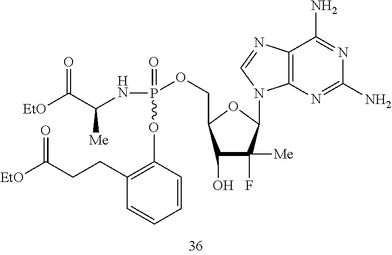

EC₅₀ = 0.7
EC₉₀ = 2.9
CC₅₀ > 10

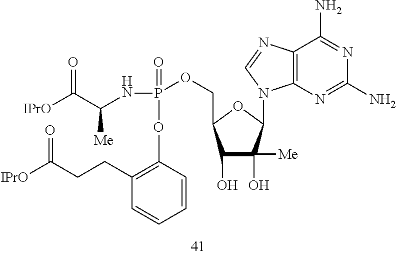

EC₅₀ = 0.6
EC₅₀ = 2.4
CC₅₀ > 10

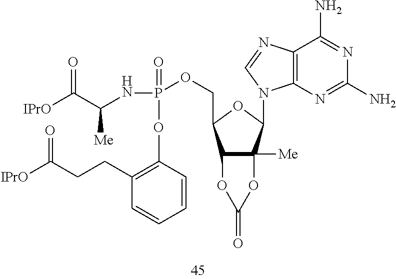

EC₅₀ = 2.8
EC₅₀ = 8.5
CC₅₀ > 10

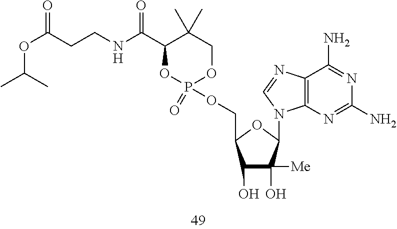

EC₅₀ > 10
EC₅₀ > 10
CC₅₀ > 10

As shown in Table 3, 8b-up was approximately 10 times more potent than 8b-down in the HCV replicon assay.

Table 4 shows the fold increase versus 1b WT across genotypes and resistant replicons at the EC₉₀*.

TABLE 4

| | | | | 2b | 3a | 4a | 5a | 1b | 1b | 1b |
|---|---|---|---|---|---|---|---|---|---|---|
| cmpd 8b-up | 1a | 1b | 2a | chimera | chimera | chimera | chimera | S282T | C316Y | M414I |
| Fold | ++ | + | ++ | ++ | + | +++ | ++ | + | ++ | +++ |
| increase | | | | | | | | | | |

+ = 0.1 to 1;
++ = 1.1 to 2;
+++ = 2.1 to 3

### REFERENCES

1. Stuyver L et al., Ribonucleoside analogue that blocks replication or bovine viral diarrhea and hepatitis C viruses in culture. Antimicrob. Agents Chemother. 2003, 47, 244-254.
2. Reed I J & Muench H, A simple method or estimating fifty percent endpoints. Am. J. Hyg. 27: 497, 1938.
3. Applied Biosystems Handbook

### Example 22

The susceptibility of West Nile virus to the compounds described herein can also be evaluated using the assay previously described in: Song, G. Y., Paul, V., Choo, H., Morrey, J., Sidwell, R. W., Schinazi, R. F., Chu, C. K. Enantiomeric synthesis of D- and L-cyclopentenyl nucleosides and their antiviral activity against HIV and West Nile virus. J. Med. Chem. 2001, 44, 3985-3993,

### Example 23

The susceptibility of Yellow fever to the compounds described herein can also be assayed as previously described in: Julander, J. G., Furuta, Y., Shafer, K., Sidwell, R. W. Activity of T-1106 in a Hamster Model of Yellow Fever Virus Infection. Antimicrob. Agents Chemother. 2007, 51, 1962-1966.

### Example 24

The susceptibility of Dengue to the compounds described herein can be evaluated using the high throughput assay disclosed by Lim et al., A scintillation proximity assay for dengue virus NS5 2′-O-methyltransferase—kinetic and inhibition analyses, Antiviral Research, Volume 80, Issue 3, December 2008, Pages 360-369.

Dengue virus (DENV) NS5 possesses methyltransferase (MTase) activity at its N-terminal amino acid sequence and is responsible for formation of a type 1 cap structure, m7 GpppAm2′-O in the viral genomic RNA. Optimal in vitro conditions for DENV2 2′-O-MTase activity can be characterized using purified recombinant protein and a short biotinylated GTP-capped RNA template. Steady-state kinetics parameters derived from initial velocities can be used to establish a robust scintillation proximity assay for compound testing. Pre-incubation studies by Lim et al., Antiviral Research, Volume 80, Issue 3, December 2008, Pages 360-369, showed that MTase-AdoMet and MTase-RNA complexes were equally catalytically competent and the enzyme supports a random bi bi kinetic mechanism. Lim validated the assay with competitive inhibitory agents, S-adenosyl-homocysteine and two homologues, sinefungin and dehydrosinefungin. A GTP-binding pocket present at the N-terminal of DENV2 MTase was previously postulated to be the cap-binding site. This assay allows rapid and highly sensitive detection of 2′-O-MTase activity and can be readily adapted for high-throughput screening for inhibitory compounds. It is also suitable for determination of enzymatic activities of a wide variety of RNA capping MTases.

### Example 25

### Anti-Norovirus Activity

Compounds can exhibit anti-norovirus activity by inhibiting norovirus polymerase and/or helicase, by inhibiting other enzymes needed in the replication cycle, or by other pathways.

There is currently no approved pharmaceutical treatment for Norovirus infection, and this has probably at least in part been due to the lack of availability of a cell culture system. Recently, a replicon system has been developed for the original Norwalk G-I strain (Chang, K. O., et al. (2006) Virology 353:463-473).

Both Norovirus replicons and Hepatitis C replicons require viral helicase, protease, and polymerase to be functional in order for replication of the replicon to occur. Most recently, an in vitro cell culture infectivity assay has been reported utilizing Norovirus genogroup I and II inoculums (Straub, T. M. et al. (2007) Emerg. Infect. Dis. 13(3):396-403). This assay is performed in a rotating-wall bioreactor utilizing small intestinal epithelial cells on microcarrier beads. The infectivity assay can be used to screen entry inhibitors.

### Example 26

### Cellular Pharmacology in HepG2 Cells

HepG2 cells are obtained from the American Type Culture Collection (Rockville, Md.), and are grown in 225 cm² tissue culture flasks in minimal essential medium supplemented with non-essential amino acids, 1% penicillin-streptomycin. The medium is renewed every three days, and the cells are subcultured once a week. After detachment of the adherent monolayer with a 10 minute exposure to 30 mL of trypsin-EDTA and three consecutive washes with medium, confluent HepG2 cells are seeded at a density of 2.5×10⁶ cells per well in a 6-well plate and exposed to 10 μM of [³H] labeled active compound (500 dpm/pmol) for the specified time periods.

The cells are maintained at 37° C. under a 5% CO₂ atmosphere. At the selected time points, the cells are washed three times with ice-cold phosphate-buffered saline (PBS).

Intracellular active compound and its respective metabolites are extracted by incubating the cell pellet overnight at −20° C. with 60% methanol followed by extraction with an additional 20 pal of cold methanol for one hour in an ice bath. The extracts are then combined, dried under gentle filtered air flow and stored at −20° C. until HPLC analysis.

### Example 27

### Cellular Pharmacology in Huh7 Cells

Similar to the method outlined for HepG2 cellular pharmacology, compounds are incubated in Huh-7 cells for 4 hr at the concentration of 50 μM in triplicate. 3TC can be used as a positive control and done in duplicate, while DMSO (10 μL) can be incubated as a blank control in duplicate. Ice-cold 70% methanol can be used as the extraction solvent. ddATP (10 nM) can be used as the internal standard.

When the parent 2,6-diamino-2′-C-Me purine nucleoside, 12, was incubated with Huh7 cells LC/MS analysis revealed extremely low levels of the corresponding 2,6-diamino-2′-C-Me purine triphosphate. The major triphosphate detected resulted from conversion of the 2,6-diamino base to the corresponding guanine analog (FIG. 6).

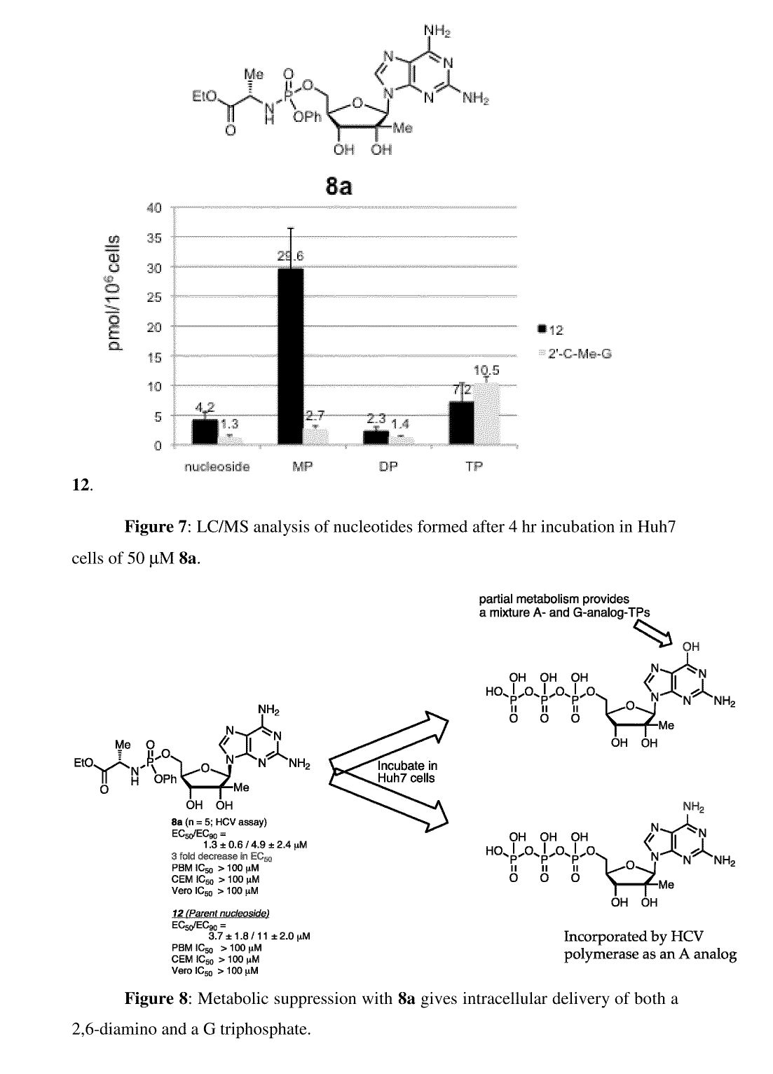

When the phosphoramidate of 12 (namely 8a) was incubated with Huh7 cells LC/MS analysis revealed unexpectedly high levels of the corresponding 2,6-diamino-2′-C-Me purine triphosphate. Furthermore, the guanine analog triphosphate was also detected (FIG. 7).

FIG. 8 shows how the phosphoramidate has unexpectedly modified the metabolic pathway of 2,6-diamino 2′-C-methyl purine, 12, and delivered 2,6-diamino-2′-C-Me purine triphosphate intracellularly at heretofore unobtainable therapeutically-relevant concentrations. In addition, the intracellular delivery of two HCV active triphosphates (one A analog and one G analog) has implications on cellular kinase saturation and resistant virus selection.

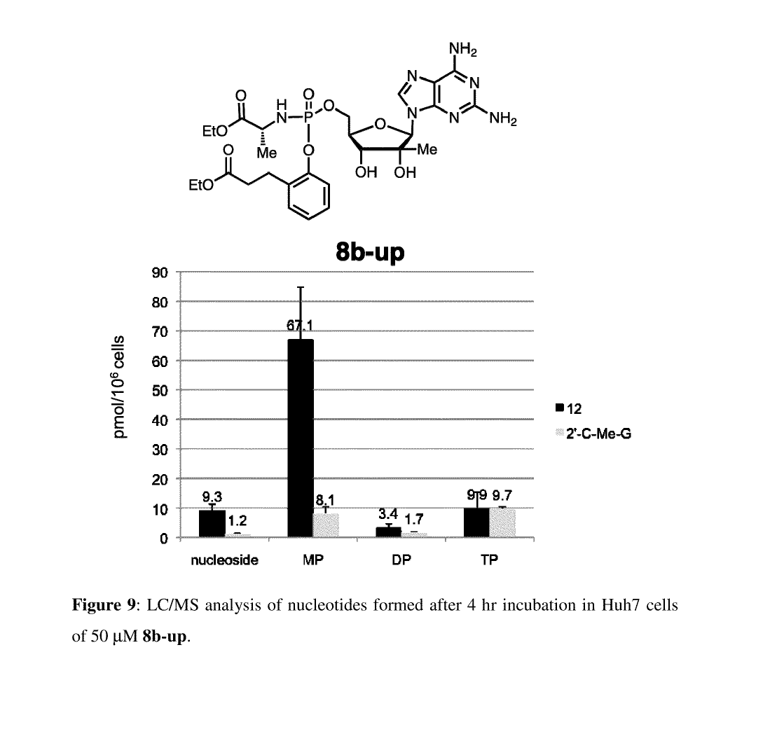

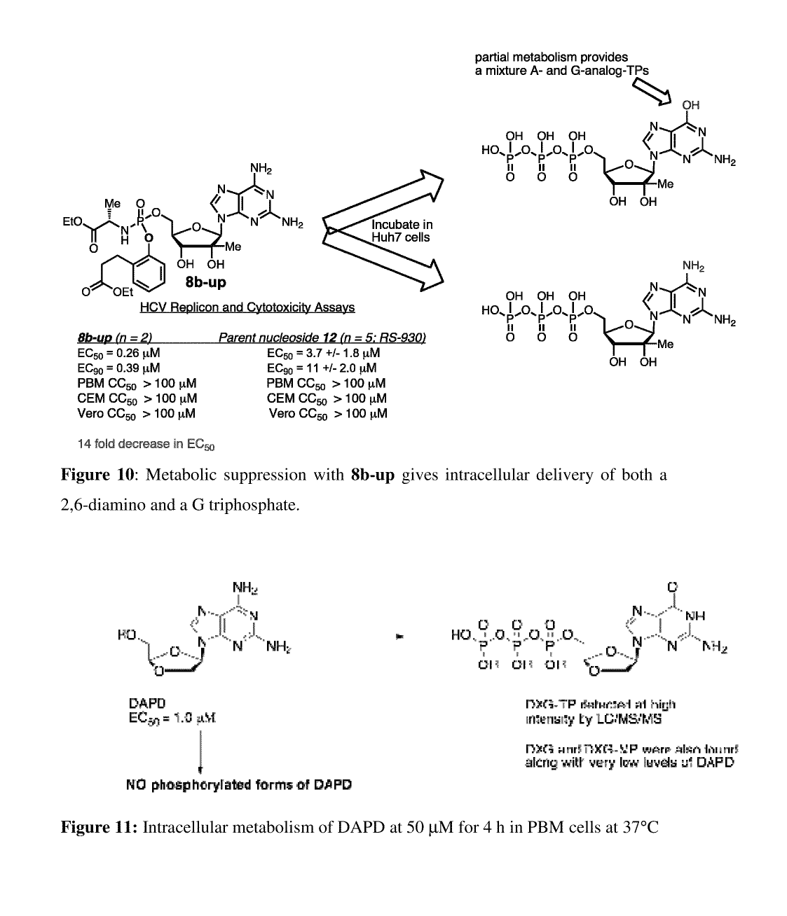

FIG. 9 shows the LC/MS analysis of nucleotides formed after 4 hr incubation in Huh7 cells with 50 μM of 8b-up. These cellular pharmacology results in Huh7 cells for 8b-up show metabolic suppression with intracellular delivery of both a 2,6-diamino and a G triphosphate (FIG. 10).

### Example 28

### Cellular Pharmacology in PBM Cells

Test compounds are incubated in PBM cells at 50 μM for 4 h at 37° C. Then the drug containing media is removed and the PBM cells are washed twice with PBS to remove extracellular drugs. The intracellular drugs are extracted from 10×10⁶ PBM cells using 1 mL 70% ice-cold methanol (containing 10 nM of the internal standard ddATP). Following precipitation, the samples are maintained at room temperature for 15 min followed by vortexing for 30 sec, and then stored 12 h at −20° C. The supernatant is then evaporated to dryness. Dry samples would be stored at −20° C. until LC-MS/MS analysis. Prior to analysis, each sample is reconstituted in 100 μL mobile phase A, and centrifuged at 20,000 g to remove insoluble particulates.

Gradient separation is performed on a Hypersil GOLD column (100×1.0 mm, 3 μm particle size; Thermo Scientific, Waltham, Mass., USA). Mobile phase A consists of 2 mM ammonium phosphate and 3 mM hexylamine. Acetonitrile is increased from 10 to 80% in 15 min, and kept at 80% for 3 min. Equilibration at 10% acetonitrile lasts 15 min. The total run time is 33 min. The flow rate is maintained at 50 μL/min and a 10 μL injection is used. The autosampler and the column compartment are typically maintained at 4.5 and 30° C., respectively.

The first 3.5 min of the analysis is diverted to waste. The mass spectrometer is operated in positive ionization mode with a spray voltage of 3.2 kV.

In the case of DAPD an even more dramatic suppression of 6-position metabolism was observed by introduction of a phosphoramidate. First, examination of the intracellular metabolism of DAPD, which contains a 6-amino group, at 50 μM for 4 h in PBM cells at 37° C. resulted in the detection of high levels of DXG-TP in addition to DXG and DXG-MP. Low levels of DAPD were observed, however, no phosphorylated forms of DAPD were detected (FIG. 11).

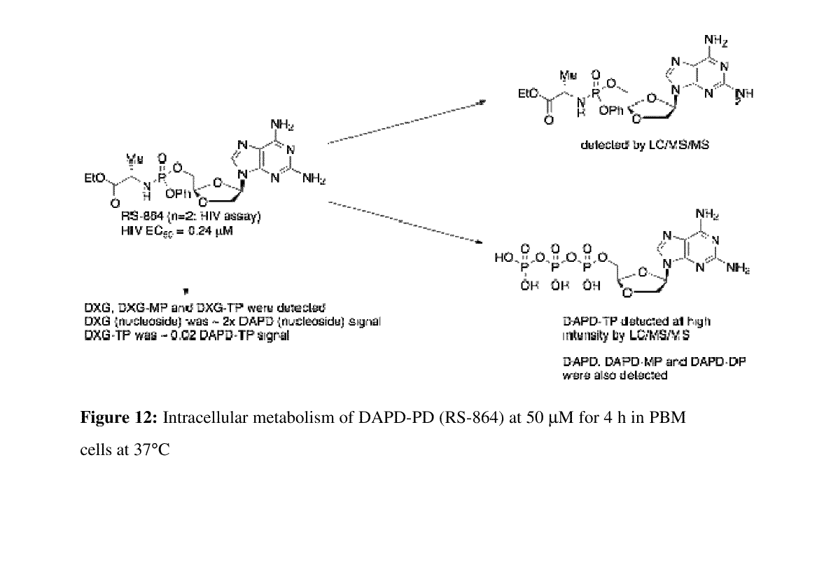

Conversely, incubation of phosphoramidate RS-864, which contains a 6-amino group and a 5′-MP prodrug, in PBM cells resulted the detection of low levels of DXG, DXG-MP, and DXG-TP (FIG. 12). However, in contrast to the incubation of DAPD, very high levels of DAPD-TP were detected. In addition, low levels of DAPD, DAPD-MP, DAPD-DP were also observed. The ratio of DXG-TP (6-OH) to DAPD-TP (6-NH₂) was approximately 2 to 98 as determined by LC/MS/MS analysis. The high levels of intercellular DAPD-TP produced upon incubation of the DAPD-MP prodrug indicate that the MP prodrug has efficiently limited or stopped the conversion of the 6-amino group to 6-OH.

### Example 29

### Bioavailability Assay in Cynomolgus Monkeys

The following procedure can be used to determine whether the compounds are bioavailable. Within 1 week prior to the study initiation, a cynomolgus monkey can be surgically implanted with a chronic venous catheter and subcutaneous venous access port (VAP) to facilitate blood collection and can undergo a physical examination including hematology and serum chemistry evaluations and the body weight recording. Each monkey (six total) receives approximately 250 μCi of ³H activity with each dose of active compound at a dose level of 10 mg/kg at a dose concentration of 5 mg/mL, either via an intravenous bolus (3 monkeys, IV), or via oral gavage (3 monkeys, PO). Each dosing syringe is weighed before dosing to gravimetrically determine the quantity of formulation administered. Urine samples are collected via pan catch at the designated intervals (approximately 18-0 hours pre-dose, 0-4, 4-8 and 8-12 hours post-dosage) and processed. Blood samples are collected as well (pre-dose, 0.25, 0.5, 1, 2, 3, 6, 8, 12 and 24 hours post-dosage) via the chronic venous catheter and VAP or from a peripheral vessel if the chronic venous catheter procedure should not be possible. The blood and urine samples are analyzed for the maximum concentration (Cmax), time when the maximum concentration is achieved (Tmax), area under the curve (AUC), half life of the dosage concentration (TV), clearance (CL), steady state volume and distribution (Vss) and bioavailability (F).

### Example 30

### Cell Protection Assay (CPA)

The assay is performed essentially as described by Baginski, S. G.; Pevear, D. C.; Seipel, M.; Sun, S. C. C.; Benetatos, C. A.; Chunduru, S. K.; Rice, C. M. and M. S. Collett “Mechanism of action of a pestivirus antiviral compound” PNAS USA 2000, 97 (14), 7981-7986. MDBK cells (ATCC) are seeded onto 96-well culture plates (4,000 cells per well) 24 hours before use. After infection with BVDV (strain NADL, ATCC) at a multiplicity of infection (MOI) of 0.02 plaque forming units (PFU) per cell, serial dilutions of test compounds are added to both infected and uninfected cells in a final concentration of 0.5% DMSO in growth medium. Each dilution is tested in quadruplicate. Cell densities and virus inocula are adjusted to ensure continuous cell growth throughout the experiment and to achieve more than 90% virus-induced cell destruction in the untreated controls after four days post-infection. After four days, plates are fixed with 50% TCA and stained with sulforhodamine B. The optical density of the wells is read in a microplate reader at 550 nm.

The 50% effective concentration (EC₅₀) values are defined as the compound concentration that achieved 50% reduction of cytopathic effect of the virus.

### Example 31

### Plaque Reduction Assay

For a compound the effective concentration is determined in duplicate 24-well plates by plaque reduction assays. Cell monolayers are infected with 100 PFU/well of virus. Then, serial dilutions of test compounds in MEM supplemented with 2% inactivated serum and 0.75% of methyl cellulose are added to the monolayers. Cultures are further incubated at 37° C. for 3 days, and then fixed with 50% ethanol and 0.8% Crystal Violet, washed and air-dried. Then plaques are counted to determine the concentration to obtain 90% virus suppression.

### Example 32

### Yield Reduction Assay

For a compound, the concentration to obtain a 6-log reduction in viral load is determined in duplicate 24-well plates by yield reduction assays. The assay is performed as described by Baginski, S. G.; Pevear, D. C.; Seipel, M.; Sun, S. C. C.; Benetatos, C. A.; Chunduru, S. K.; Rice, C. M. and M. S. Collett “Mechanism of action of a pestivirus antiviral compound” PNAS USA 2000, 97 (14), 7981-7986, with minor modifications.

Briefly, MDBK cells are seeded onto 24-well plates (2×10⁵ cells per well) 24 hours before infection with BVDV (NADL strain) at a multiplicity of infection (MOI) of 0.1 PFU per cell. Serial dilutions of test compounds are added to cells in a final concentration of 0.5% DMSO in growth medium. Each dilution is tested in triplicate. After three days, cell cultures (cell monolayers and supernatants) are lysed by three freeze-thaw cycles, and virus yield is quantified by plaque assay. Briefly, MDBK cells are seeded onto 6-well plates (5×10⁵ cells per well) 24 h before use. Cells are inoculated with 0.2 mL of test lysates for 1 hour, washed and overlaid with 0.5% agarose in growth medium. After 3 days, cell monolayers are fixed with 3.5% formaldehyde and stained with 1% crystal violet (w/v in 50% ethanol) to visualize plaques. The plaques are counted to determine the concentration to obtain a 6-log reduction in viral load.

### Example 33

### Diagnosis of Norovirus Infection

One can diagnose a norovirus infection by detecting viral RNA in the stools of affected persons, using reverse transcription-polymerase chain reaction (RT-PCR) assays. The virus can be identified from stool specimens taken within 48 to 72 hours after onset of symptoms, although one can obtain satisfactory results using RT-PCR on samples taken as long as 7 days after the onset of symptoms. Other diagnostic methods include electron microscopy and serologic assays for a rise in titer in paired sera collected at least three weeks apart. There are also commercial enzyme-linked immunoassays available, but these tend to have relatively low sensitivity, limiting their use to diagnosis of the etiology of outbreaks. Clinical diagnosis of norovirus infection is often used, particularly when other causative agents of gastroenteritis have been ruled out.

### Example 34

### In Vitro Anti-Viral Activity

In vitro anti-viral activity can be evaluated in the following cell lines:

The Norwalk G-I strain (Chang, K. O., et al. (2006) Virology 353:463-473), the GII-4 strain replicon, as well other Norovirus replicons can be used in assays to determine the in vitro antiviral activity of the compounds described herein, or other compounds or compound libraries. In some embodiments, the replicon systems are subgenomic and therefore allow evaluation of small molecule inhibitors of non-structural proteins. This can provide the same benefits to Norovirus drug discovery that Hepatitis C replicons contributed to the discovery of therapeutics useful for treatment of that virus (Stuyver, L. J., et al. (2006) Antimicrob. Agents Chemother. 47:244-254). Both Norovirus replicons and Hepatitis C replicons require viral helicase, protease, and polymerase to be functional in order for replication of the replicon to occur. It is believed that the compounds described herein inhibit viral polymerase and/or viral helicase.

The in vitro cell culture infectivity assay reported using Norovirus genogroup I and II inoculums (Straub, T. M. et al. (2007) Emerg. Infect. Dis. 13(3):396-403) can also be used. This assay can be performed in a rotating-wall bioreactor utilizing small intestinal epithelial cells on microcarrier beads. The infectivity assay can be used for screening compounds for their ability to inhibit the desired virus.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

1. A compound of Formula (A) or a compound of Formula (B): 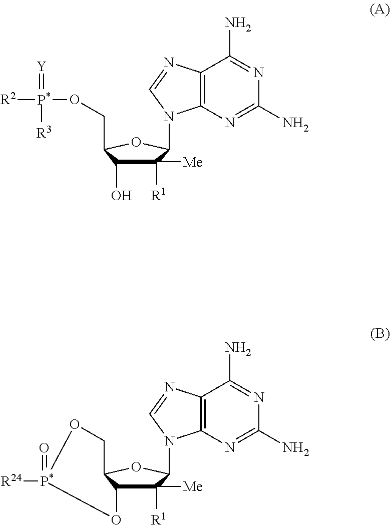
or a pharmaceutically acceptable salt or prodrug thereof, wherein:
when chirality exists at the phosphorous center it may be wholly or partially Rₚ or Sₚ or any mixture thereof
R¹ is OH or F;
Y is O or S;
R²⁴ is selected from OR¹⁵, 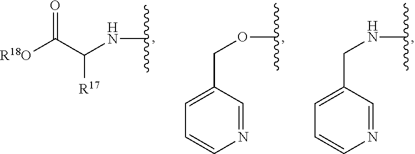
and fatty alcohols,
wherein R¹⁵, R¹⁷, and R¹⁸ are as defined below;
R² and R³, when administered in vivo, are capable of providing the nucleoside monophosphate or thiomonophosphate that is either partially or fully resistant to 6-NH₂ deamination in a biological system and are independently selected from the group consisting of:
(a) OR¹⁵ where R¹⁵ selected from H, Li, Na, K, phenyl and pyridinyl; Phenyl and pyridinyl are substituted with one to three substituents independently selected from the group consisting of (CH₂)₀₋₆CO₂R¹⁶ and (CH₂)₀₋₆CON(R¹⁶)₂;
R¹⁶ is independently H, C₁₋₂₀ alkyl, the carbon chain derived from a fatty alcohol or C₁₋₂₀ alkyl substituted with a lower alkyl, alkoxy, di(lower alkyl)-amino, fluoro, C₃₋₁₀ cycloalkyl, cycloalkyl alkyl, cycloheteroalkyl, aryl, heteroaryl, substituted aryl, or substituted heteroaryl; wherein the substituents are C₁₋₅ alkyl, or C₁₋₅ alkyl substituted with a lower alkyl, alkoxy, di(lower alkyl)-amino, fluoro, C₃₋₁₀ cycloalkyl, or cycloalkyl; 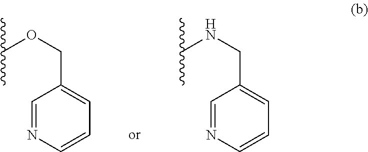 [SMILES: *OCc1cccnc1 ; *NCc1cccnc1]
(c) the ester of an L-amino acid 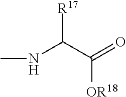
where R¹⁷ is restricted to those occurring in natural L-amino acids, and R¹⁸ is H, C₁₋₂₀ alkyl, the carbon chain derived from a fatty alcohol or C₁₋₂₀ alkyl substituted with a lower alkyl, alkoxy, di(lower alkyl)-amino, fluoro, C₃₋₁₀ cycloalkyl, cycloalkyl alkyl, cycloheteroalkyl, aryl, heteroaryl, substituted aryl, or substituted heteroaryl; wherein the substituents are C₁₋₅ alkyl, or C₁₋₅ alkyl substituted with a lower alkyl, alkoxy, di(lower alkyl)-amino, fluoro, C₃₋₁₀ cycloalkyl, or cycloalkyl;
(d) R² and R³ together to form a ring 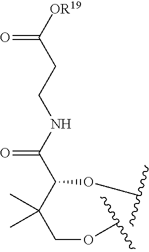
where R¹⁹ is H, C₁₋₂₀ alkyl, C₁₋₂₀ alkenyl, the carbon chain derived from a fatty alcohol or C₁₋₂₀ alkyl substituted with a lower alkyl, alkoxy, di(lower alkyl)-amino, fluoro, C₃₋₁₀ cycloalkyl, cycloalkyl alkyl, cycloheteroalkyl, aryl, heteroaryl, substituted aryl, or substituted heteroaryl; wherein the substituents are C₁₋₅ alkyl, or C₁₋₅ alkyl substituted with a lower alkyl, alkoxy, di(lower alkyl)-amino, fluoro, C₃₋₁₀ cycloalkyl, or cycloalkyl; and
(e) R² and R³ together to form a ring selected from 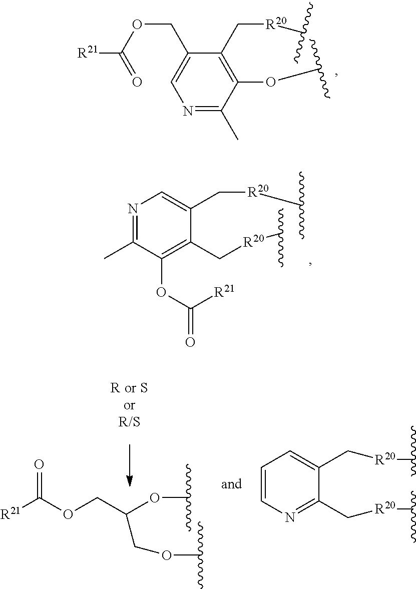
where R²⁰ is O or NH and
R²¹ is selected from H, C₁₋₂₀ alkyl, C₁₋₂₀ alkenyl, the carbon chain derived from a fatty acid, and C₁₋₂₀ alkyl substituted with a lower alkyl, alkoxy, di(lower alkyl)-amino, fluoro, C₃₋₁₀ cycloalkyl, cycloalkyl alkyl, cycloheteroalkyl, aryl, heteroaryl, substituted aryl, or substituted heteroaryl; wherein the substituents are C₁₋₅ alkyl, or C₁₋₅ alkyl substituted with a lower alkyl, alkoxy, di(lower alkyl)-amino, fluoro, C₃₋₁₀ cycloalkyl, or cycloalkyl.

2. The compounds of claim 1, wherein the compounds are in the β-D configuration.

3. The compounds of claim 1, wherein the compounds are converted in a biological system to mixture C or D of 6-NH₂ and 6-OH purine triphosphates.

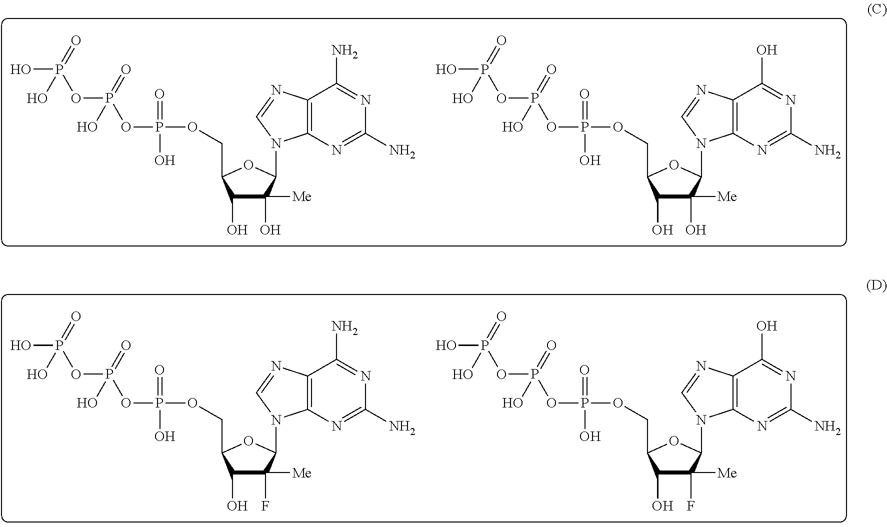

4. The compounds of claim 1, wherein the compounds are converted in a biological system to therapeutically-relevant concentrations of 2,6-diamino 2′-C-methyl purine triphosphate, E or 2,6-diamino 2′-C-methyl 2′-deoxy 2′-fluoro purine triphosphate, F.

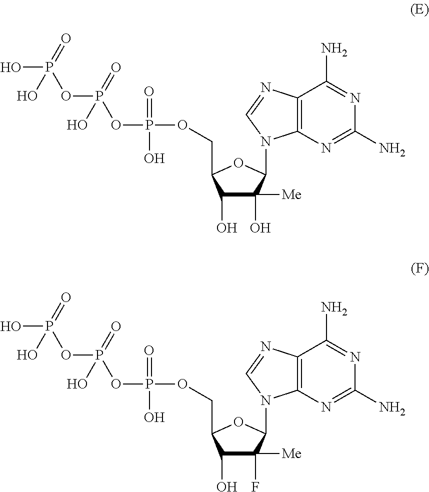

5. A compound of claim 1 of the formula: 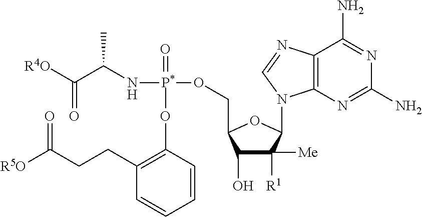
wherein R¹ is as defined in claim 1, and R⁴ is C₁₋₆ alkyl or a carbon chain derived from a fatty alcohol.

6. The compound of claim 5, wherein the values of R¹, R⁴, and R⁵ are selected as follows:

| R¹ | R⁴ | R⁵ |
|---|---|---|
| OH | Me | Me |
| F | Me | Me |
| OH | Et | Et |
| F | Et | Et |
| OH | i-Pr | i-Pr |
| F | i-Pr | i-Pr |
| OH | oleyl | oleyl |
| F | oleyl | oleyl |

7. A compound of claim 1 of the formula: 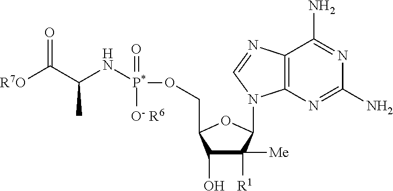
wherein R¹ is as defined in claim 1, R⁶ is H or an alkali metal, and R⁷ a carbon chain derived from a fatty alcohol.

8. A compound of claim 7, wherein the values for R¹, R⁶, and R⁷ are as provided below:

| R¹ | R⁶ | R⁷ |
|---|---|---|
| OH | Na⁺ | linoleyl |
| F | Na | linoleyl |
| OH | K | linoleyl |
| F | K | linoleyl |
| OH | Na | oleyl |
| F | Na | oleyl |
| OH | K | oleyl |
| F | K | oleyl |

9. A compound of claim 1 of the formula: 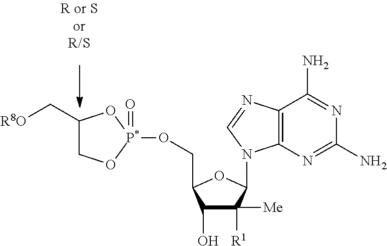
wherein R¹ is as defined in claim 1, and R⁸ is a fatty acid radical.

10. A compound of claim 9, wherein the values for R¹ and R⁸ are as provided below:

| R¹ | R⁸ |
|---|---|
| OH | linoleyl |
| F | linoleyl |
| OH | oleyl |
| F | oleyl |

11. A compound of claim 1 of the formulas: 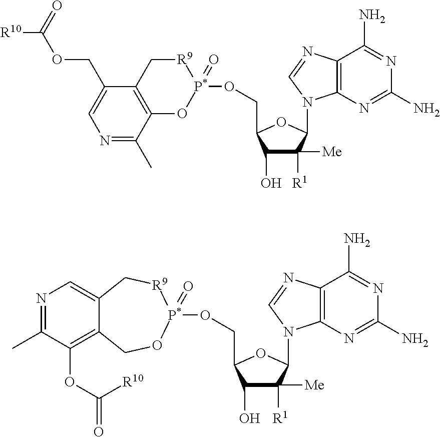
wherein R¹ is as defined in claim 1, and R⁹ is O or NH, and R¹⁰ is a C₁₋₆ alkyl or a carbon chain derived from a fatty alcohol.

12. A compound of claim 11, wherein the values for R¹, R⁹, and R¹⁰ are as provided below:

| R¹ | R⁹ | R¹⁰ |
|---|---|---|
| OH | O | Me |
| F | O | Me |
| OH | NH | Me |
| F | NH | Me |
| OH | O | Et |
| F | O | Et |
| OH | NH | Et |
| F | NH | Et |
| OH | O | i-Pr |
| F | O | i-Pr |
| OH | NH | i-Pr |
| F | NH | i-Pr |

13. A compound of claim 1 having the formulas: 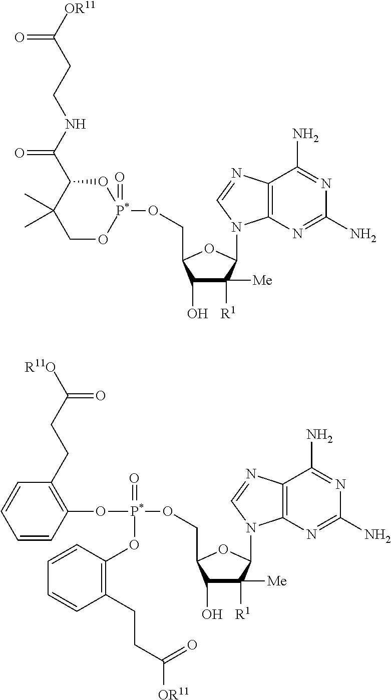
wherein R¹ is as defined in claim 1, and R¹¹ is a C₁₋₆ alkyl or a carbon chain derived from a fatty alcohol.

14. A compound of claim 13, wherein the values of R¹ and R¹¹ are as provided below:

| R¹ | R¹¹ |
|---|---|
| OH | Me |
| F | Me |
| OH | Et |
| F | Et |
| OH | i-Pr |
| F | i-Pr |

15. A compound of claim 1 of the formulas: 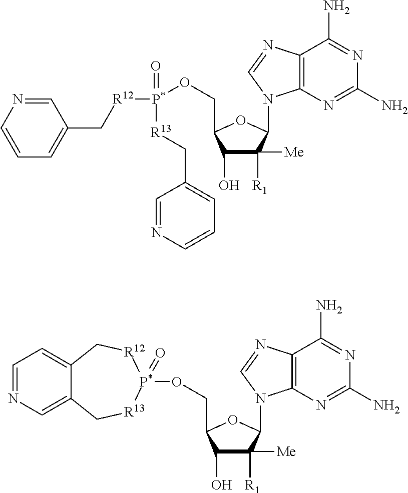
wherein R¹ is as defined in claim 1, and R¹² and R¹³ are, independently, O or NH.

16. A compound of claim 15, wherein the values of R¹, R¹², and R¹³ are as provided below:

| R¹ | R¹² | R¹³ |
|---|---|---|
| OH | O | O |
| F | O | O |
| OH | O | NH |
| F | O | NH |
| OH | NH | NH |
| F | NH | NH |

17. A compound of claim 1 having the formula: 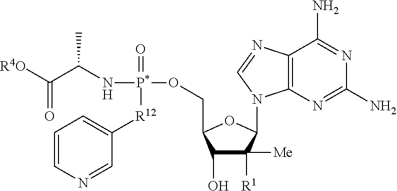
wherein R¹ is as defined in claim 1, R⁴ is C₁₋₆ alkyl or a carbon chain derived from a fatty alcohol, and R¹² is O or NH.

18. A compound of claim 17, wherein the values of R¹, R⁴, and R¹² are as provided below:

| R¹ | R⁴ | R¹² |
|---|---|---|
| OH | Me | O |
| F | Me | O |
| OH | Et | O |
| F | Et | O |
| OH | i-Pr | O |
| F | i-Pr | O |
| OH | oleyl | O |
| OH | Me | NH |
| F | Me | NH |
| OH | Et | NH |
| F | Et | NH |
| OH | i-Pr | NH |
| F | i-Pr | NH |
| OH | oleyl | NH |
| F | oleyl | NH |

19. A compound of claim 1 of the formula: 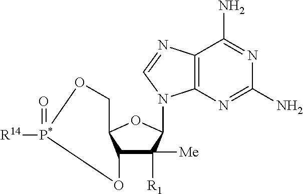
wherein 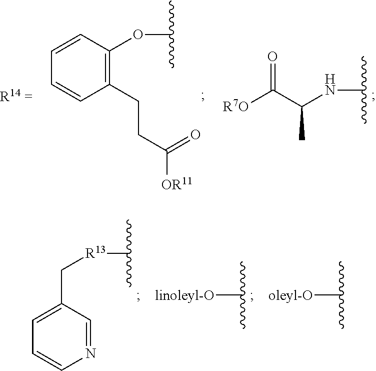
and R¹¹, R⁷ and R¹³ are as defined above.

20. A process for preparing compounds of claim 1 wherein the phosphorous-5′-oxygen bond is formed by reaction with a reagent of general formulas G or H: 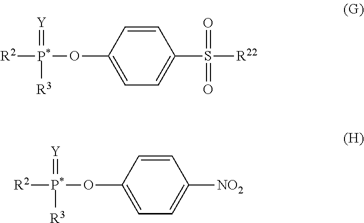
wherein:
the chirality at the phosphorous center of formulas G or H can be wholly or partially Rₚ or Sₚ or any mixture thereof,
Y, R² and R³ are as defined above, and
R²² is, independently, H, C₁₋₂₀ alkyl, CF₃, aryl, heteroaryl, substituted aryl, or substituted heteroaryl, or C₁₋₂₀ alkyl substituted with a lower alkyl, alkoxy, di(lower alkyl)-amino, chloro, fluoro, aryl, such as phenyl, heteroaryl, substituted aryl, or substituted heteroaryl.

21-32. (canceled)

33. A method for treating a host infected with Flaviviridae family of viruses, or for reducing the biological activity of an infection with Flaviviridae family of viruses, comprising administering an effective amount of a compound of claim 1 to a patient in need of treatment thereof.

34-35. (canceled)

36. The method of claim 33, wherein the compound is administered in combination with another anti-Flaviviridae virus agent.

37. (canceled)

38. A pharmaceutical composition comprising a compound of claim 1, and a pharmaceutically-acceptable carrier.

39. The method of claim 34, for treating a host infected with wherein the Flaviviridae virus is Norovirus or Saporovirus, comprising administering an effective amount of a compound of claim 1 to a patient in need of treatment thereof.

40-43. (canceled)

44. The pharmaceutical composition of claim 38, further comprising a second antiviral agent.

45. The pharmaceutical composition of claim 44, wherein the second antiviral agent is selected from the group consisting of an interferon, ribavirin, an NS3 protease inhibitor, an NS5A inhibitor, a non-nucleoside polymerase inhibitor, a helicase inhibitor, a polymerase inhibitor, a nucleotide or nucleoside analogue, an inhibitor of IRES dependent translation, and combinations thereof.
